(19) United States
(12) Patent Application Publication
(10) Pub. No.: US 2024/0277698 A1
(43) Pub. Date: Aug. 22, 2024

(54) METHODS OF USING ALDOSTERONE SYNTHASE INHIBITORS

(71) Applicant: CINCOR PHARMA, INC., CINCINNATI, OH (US)

(72) Inventors: Mary BOND, West Chester, PA (US); Brian MURPHY, Cincinnati, OH (US); Catherine PEARCE, Cincinnati, OH (US)

(21) Appl. No.: 18/569,853

(22) PCT Filed: Jun. 24, 2022
(86) PCT No.: PCT/US2022/073148

§ 371 Date: Dec. 13, 2023

Related U.S. Application Data
(60) Provisional application No. 63/290,364, filed on Dec. 16, 2021.
(60) Provisional application No. 63/214,521, filed on Jun. 24, 2021.

(51) Int. Cl. A61K 31/4725; A61P 9/12

(52) U.S. Cl. CPC A61K 31/4725; A61P 9/12

(57) ABSTRACT

The disclosure provides methods of inhibiting human aldosterone synthase, treating hypertension, or treating primary aldosteronism in a subject in need thereof, comprising administering an effective amount of (R)-Compound 1 to the subject, wherein (R)-Compound 1 is: Formula (I).

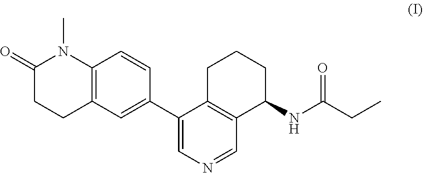 [SMILES: CCC(=O)N[C@@H]1CCCc2c(-c3ccc4c(c3)CCC(=O)N4C)cncc21]

### CROSS REFERENCE TO RELATED APPLICATIONS

This application claims the benefit of U.S. Patent Provisional Application No. 63/214,521, filed Jun. 24, 2021, and U.S. Provisional Patent Application No. 63/290,364, filed Dec. 16, 2021, the entireties of which are incorporated by reference herein.

### TECHNICAL FIELD

The disclosure provides compounds and methods of treating hypertension or primary aldosteronism.

### BACKGROUND

Aldosterone is a hormone that has been implicated in a variety of cardiovascular and renal diseases. It is the principal mineralocorticoid in humans and is synthesized in the adrenal cortex by aldosterone synthase. It is a key component of the renin-angiotensin-aldosterone system (RAAS) and acts as a critical regulator of fluid and electrolyte homeostasis through its agonism of the mineralocorticoid receptor (MR). Aldosterone's effect on end organs has been shown to occur via its direct interaction with the MR (genomic effect) in addition to mechanisms independent of that direct interaction (non-genomic or non-receptor mediated effects).

Blood pressure (BP) is significantly reduced by partially inhibiting the activity of the RAAS with angiotensin-converting enzyme inhibitors (ACEIs), angiotensin receptor blockers (ARBs), direct renin inhibitors, or MR antagonists (MRAs). The mechanism of action of these agents involves a reduction in aldosterone levels. These effects are demonstrated to occur in the setting of both normal and inappropriately elevated aldosterone levels. Many patients with hypertension have inappropriately high aldosterone concentrations that promote cardiac, renal, and vascular injury. Inhibiting aldosterone synthesis represents a promising target for the reduction of BP and mitigation of BP-dependent target organ damage. The association between plasma aldosterone and long-term survival has been demonstrated in patients with congestive heart failure, acute myocardial infarction, and coronary artery diseases outside the setting of heart failure or acute myocardial infarction. The blockade of aldosterone thereby represents a means not only to reduce BP, but also to mitigate target organ damage. Therefore, directly inhibiting the synthesis of aldosterone represents a promising target for the reduction of BP and a mitigation of the genomic and non-genomic effects on end organ damage.

One of the challenges that has impacted the development of aldosterone synthase inhibitors (ASIs) has been the difficulty in selectively inhibiting aldosterone synthase and not affecting the synthesis of cortisol. The synthesis pathway of cortisol is catalyzed by 11β-hydroxylase (encoded by the cytochrome P450 family 11 subfamily B member 1 [CYP11B1] gene) and shares high sequence homology with aldosterone synthase (encoded by the CYP11B2 gene). Undesired inhibition of 11β-hydroxylase leads to suppression of cortisol levels, compromised stress and immunologic responses, adverse effects on some metabolic functions, and possibly increased mortality rates. LCI699, an ASI, was taken into clinical trials by Novartis but was discontinued for both anti-hypertensive and primary aldosteronism indications due to its lack of specificity for aldosterone synthase.

Compound 1 is a highly potent, selective, and competitive inhibitor of human aldosterone synthase. In preclinical in-vivo studies (primarily conducted in primates), Compound 1 significantly lowered aldosterone without affecting cortisol levels over a wide dose range. Methods of using Compound 1 to safety and effectively treat humans are needed.

### SUMMARY

The disclosure provides methods of treating hypertension or primary aldosteronism in a human, comprising administering 0.1 to 10 mg/day of (R)-Compound 1 to the human:

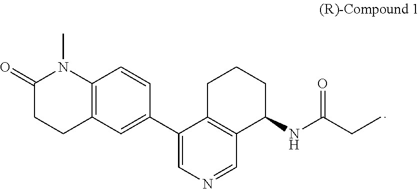 [SMILES: CCC(=O)N[C@@H]1CCCc2c(-c3ccc4c(c3)CCC(=O)N4C)cncc21]

Other aspects and embodiments of the invention will be readily apparent from the following detailed description of the invention.

### BRIEF DESCRIPTION OF THE DRAWINGS

The foregoing summary, as well as the following detailed description, will be better understood when read in conjunction with the appended drawings, which show exemplary embodiments for the purposes of illustration.

FIG. 1 depicts a plot of mean (+standard deviation) plasma (R)-compound 1 concentrations versus time (Day 10, 0-24 Hours, after repeated once-daily dosing) by treatment on linear scale—pharmacokinetic population. In this figure, the lower limit of quantitation for the (R)-compound 1=0.05 ng/ml. Actual sampling times that were outside of the analysis sampling time window were excluded in the mean plot.

FIG. 2 depicts a plot of Cₘₐₓ versus (R)-compound 1 dose (day 10 after repeated dosing—pharmacokinetic population). In this figure, the solid line represents the predicted values and the dashed lines represent the 90% confidence intervals around the regression line. The black dots represent the geometric mean of the PK parameter.

FIG. 3 depicts a plot of AUC₀₋ₜₐᵤ versus (R)-compound 1 dose (day 10 after repeated dosing—pharmacokinetic population). In this figure, the solid line represents the predicted values and the dashed lines represent the 90% confidence intervals around the regression line. The black dots represent the geometric mean of the PK parameter. AUC₀₋ₜₐᵤ is the area under the plasma concentration-time curve over a dosing interval.

FIG. 4 depicts a plot of mean (standard deviation) plasma (R)-compound 1 concentrations versus time (day 1, Single Dose) by treatment on linear scale—pharmacokinetic population.

FIG. 5 depicts a plot of mean aldosterone plasma concentration over time by treatment for normal salt diet treatment groups—pharmacodynamic population (excluding outlier subjects).

FIG. 6 depicts a plot of mean aldosterone plasma concentration over time by treatment for low salt diet treatment groups—pharmacodynamic population (excluding outlier subjects).

FIG. 7 depicts a mean plasma concentration versus time profile of (R)-compound 1 following single and multiple oral doses of (R)-compound 1 (a) SAD study, (b) MAD study.

FIG. 8 depicts a mean plasma concentration versus time profiles of (R)-compound 1 following a single intravenous dose and a single oral dose of 3 mg (R)-compound 1.

FIG. 9 depicts a mean (+SD) plasma concentration of (R)-compound 1 versus time (0 to 24 Hours) profile following single-dose administration of (R)-compound 1 oral solution and tablet.

FIG. 10 depicts a mean aldosterone plasma concentrations versus time by dose group following administration of a single-dose of (R)-compound 1 or placebo.

FIG. 11 depicts a mean change from baseline in aldosterone plasma concentrations versus time by dose group following multiple-dose administration of (R)-compound 1 or placebo.

FIG. 12 depicts a mean change from baseline in aldosterone plasma concentrations versus time by dose group following multiple-dose administration of (R)-compound 1 or placebo.

FIG. 13 depicts a plot of mean (+SD) plasma metformin concentrations by treatment on a linear scale to hour 24-PK population. In this figure, LLOQ (lower limit of quantification) for metformin is 0.5 ng/mL. Treatment A is a single 1000 mg dose of immediate-release metformin was administered and treatment B is a single 1000 mg dose of immediate release metformin was coadministered with a 10 mg dose of Compound 1. Scheduled time point is shown as relative to metformin dosing.

FIG. 14 depicts a plot of mean (+SD) Ae of metformin by treatment on a linear scale to hour 24-PK population. In this figure, treatment A is a single 1000 mg dose of immediate-release metformin was administered and treatment B is a single 1000 mg dose of immediate release metformin was coadministered with a 10 mg dose of Compound 1. Ae is the cumulative amount of drug excreted in the urine.

FIG. 15 depicts plots of mean (+SD) plasma metformin concentrations by treatment on linear and semi-logarithmic scales to hour 24-PK population. In this figure, LLOQ for metformin is 0.5 ng/mL. Treatment A is a single 1000 mg dose of immediate-release metformin was administered. Treatment B is a single 1000 mg dose of immediate release metformin was coadministered with a 10 mg dose of Compound 1. Scheduled time point is shown as relative to metformin dosing.

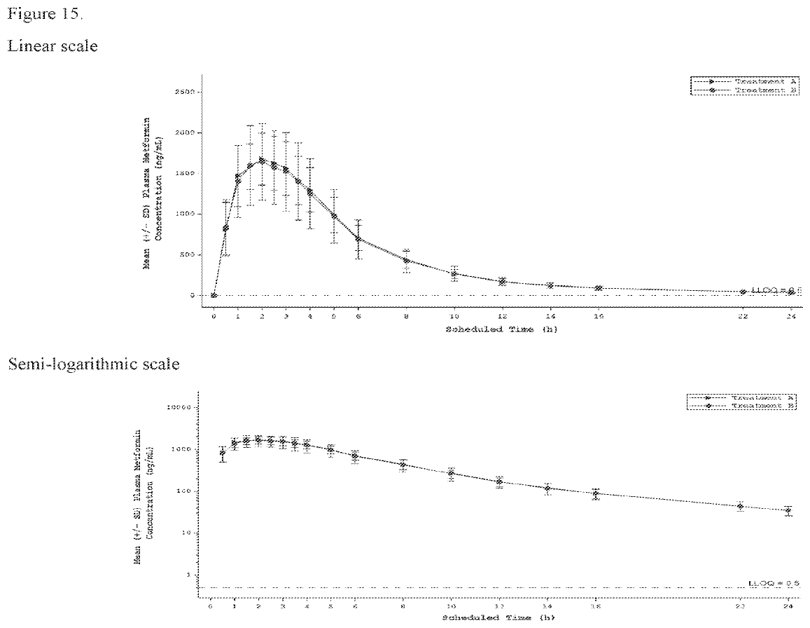

FIG. 16 depicts a plot of mean (+SD) Ae of metformin by treatment on a linear scale to hour 24-PK population. In this figure, treatment A is a single 1000 mg dose of immediate-release metformin was administered and treatment B is a single 1000 mg dose of immediate release metformin was coadministered with a 10 mg dose of Compound 1. Ae is the cumulative amount of drug excreted in the urine.

FIG. 17 depicts a plot of mean (±SD) Ae of metformin by treatment on a linear scale—PK population, extended to hour 72. In this figure, treatment A is a single 1000 mg dose of immediate-release metformin was administered and treatment B is a single 1000 mg dose of immediate release metformin was coadministered with a 10 mg dose of Compound 1. Ae is the cumulative amount of drug excreted in the urine.

FIG. 18 depicts plots of mean (±SD) plasma Compound 1 concentrations by treatment on linear and semi-logarithmic scales to hour 24—PK population. In this figure, LLOQ for Compound 1 is 5 ng/mL. Treatment B is a single 1000 mg dose of immediate-release metformin was coadministered with a 10 mg dose of Compound 1. Scheduled time point is shown as relative to Compound 1 dosing, which occurred 2 hours prior to metformin dosing.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination. Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

In the disclosure, the singular forms “a,” “an,” and “the” include the plural reference, and reference to a particular numerical value includes at least that particular value, unless the context clearly indicates otherwise. Thus, e.g., a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude an optional element. As such, this statement is intended to serve as antecedent basis for use of such exclusive terminology as “only” and the like in connection with the recitation of claim elements, or use of a “negative” limitation. Finally, while an embodiment may be described as part of a series of steps or part of a more general structure, each said step may also be considered an independent embodiment in itself.

The present disclosure provides methods of treating hypertension or primary aldosteronism in a human comprising administering 0.1 to 30 mg/day, for example, 0.1 to 25 mg/day, 0.1 to 20 mg/day, 0.1 to 15 mg/day, 0.1 to 10 mg/day, or 0.5 to 10 mg/day, of Compound 1 or (R)-Compound 1 to the human. In some aspects, hypertension or primary aldosteronism in a human is treated by administering to the human 0.1 to 20 mg/day, 0.1 to 15 mg/day, 0.1 to 10 mg/day, or 0.5 to 10 mg/day of (R)-Compound 1. In some aspects, hypertension or primary aldosteronism in a human is treated by administering to the human 0.1 to 10 mg/day or 0.5 to 10 mg/day of (R)-Compound 1.

The terms “subject” and “patient” are used interchangeably and typically refer to mammals. In some embodiments, the patient or subject is a human. In other embodiments, the patient or subject is a veterinary or farm animal, a domestic animal or pet, or animal used for conducting clinical research. In some embodiments, the subject or patient is at least 18 years of age, i.e., an adult.

The terms “hypertension” and “high blood pressure” are used interchangeably and refer to a condition where a patient's blood pressure is consistently about 130/80 mm Hg or greater. Hypertension includes stages 1 and 2 hypertension and hypertensive crisis. In some embodiments, the patient has stage 1 hypertension with a systolic pressure of about 130 to about 139 mm Hg and/or a diastolic pressure of about 80 to about 89 mm Hg. In other embodiments, the patient has stage 2 hypertension with a systolic pressure of about 140 mm Hg or higher and/or a diastolic pressure of about 90 mm Hg or higher. In further embodiments, the patient has hypertensive crisis with a blood pressure measurement higher than about 180/120 mm Hg.

The term “primary aldosteronism” and “hyperaldosteronism” are used interchangeably and refer to a condition that occurs when the adrenal glands produce too much aldosterone. In some embodiments, primary aldosteronism results in elevated blood pressures. Prior to administering Compound 1 or (R)-Compound 1, the subject or patient has a blood pressure of ≥130/80 mmHg. In some embodiments, the subject or patient has a mean seated blood pressure of ≥130/80 mmHg. In some embodiments, the patient is in a fasted state. In other embodiments, the patient is in a fed state. The term “fasted state” as used herein refers to the absence of food consumption by the patient prior to administering Compound 1 or (R)-Compound 1. In some embodiments, the patient in a “fasted state” if no food is consumed at least about 8 hours before administering Compound 1 or (R)-Compound 1. The term “fasted state” may also include refraining from eating after administering Compound 1 or (R)-Compound 1. In further embodiments, the patient is in a “fasted state” if no food is consumed for about 4 hours after administering Compound 1 or (R)-Compound 1.

“Treating” or variations thereof refers to eliminating or reducing at least one physical parameter of a disease or disorder, such as hypertension or primary aldosteronism.

In some embodiments, the disease or disorder is hypertension. In other embodiments, the disease or disorder is primary aldosteronism.

Compound 1 as used herein refers to N-(4-(1-methyl-2-oxo-1,2,3,4-tetrahydroquinolin-6-yl)-5,6,7,8-tetrahydroisoquinolin-8-yl)propionamide having the following structure:

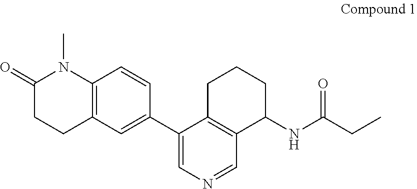 [SMILES: CCC(=O)NC1CCCc2c(-c3ccc4c(c3)CCC(=O)N4C)cncc21]

In some embodiments, Compound 1 is (R)—N-(4-(1-methyl-2-oxo-1,2,3,4-tetrahydroquinolin-6-yl)-5,6,7,8-tetrahydroisoquinolin-8-yl)propionamide having the following structure:

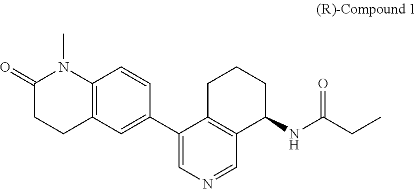 [SMILES: CCC(=O)N[C@@H]1CCCc2c(-c3ccc4c(c3)CCC(=O)N4C)cncc21]

In some aspects, a mixture of enantiomers of Compound 1 is administered to the human. In other aspects, a racemic mixture of enantiomers of Compound 1 (i.e., (R,S)-Compound 1) is administered to the human. In further aspects, (R)-Compound 1 having an enantiomeric purity of 50% enantiomeric excess (ee) or greater is administered to the human. In yet other aspects, (R)-Compound 1 having an enantiomeric purity of 60% ee or greater is administered to the human. In still further aspects, (R)-Compound 1 having an enantiomeric purity of 70% ee or greater is administered to the human. In other aspects, (R)-Compound 1 having an enantiomeric purity of 80% ee or greater is administered to the human. In further aspects, (R)-Compound 1 having an enantiomeric purity of 90% ee or greater is administered to the human. In yet other aspects, (R)-Compound 1 having an enantiomeric purity of 95% ee or greater is administered to the human. In still further aspects, (R)-Compound 1 having an enantiomeric purity of 98% ee or greater is administered to the human. In other aspects, (R)-Compound 1 having an enantiomeric purity of 99% ee or greater is administered to the human.

The disclosure also contemplates salts of Compound 1 such as salts of (R)-Compound 1. In some embodiments, the salt is pharmaceutically acceptable. “Pharmaceutically acceptable” refers to properties and/or substances that are acceptable to the patient from a pharmacological/toxicological vantage, and to the manufacturing pharmaceutical chemist from a physical/chemical vantage regarding composition, formulation, stability, patient acceptance, and bioavailability.

A pharmaceutically acceptable salt of Compound 1 or (R)-Compound 1 includes salts with a pharmaceutically acceptable acid or base, e.g., inorganic acids, e.g., hydrochloric, sulfuric, phosphoric, diphosphoric, hydrobromic, hydroiodic, nitric, and phosphoric acid and organic acids, i.e., adipic, citric, fumaric, maleic, malic, malonic, mandelic, ascorbic, oxalic, succinic, tartaric, benzoic, acetic, methanesulphonic, ethanesulphonic, benzenesulphonic, cyclohexylsulfamic (cyclamic), edisylate, glutaric, or p-toluenesulfonic acid. Pharmaceutically acceptable bases include alkali metal, e.g., sodium or potassium, and alkali earth metal, e.g., calcium or magnesium, hydroxides, and organic bases, e.g., alkyl amines, arylalkyl amines and heterocyclic amines.

The disclosure further provides hydrates and/or polymorphs of Compound 1. In some embodiments, Compound 1 is a hydrate. In other embodiments, Compound is a monohydrate. In other embodiments, Compound 1 is in an anhydrous form.

Compound 1 may also be in crystalline and/or amorphous forms. In some embodiments, Compound 1 in an amorphous form. In other embodiments, Compound 1 is in a crystalline form.

The disclosure also provides pharmaceutical compositions comprising Compound 1 or (R)-Compound 1 and one or more pharmaceutically acceptable excipients. In some embodiments, the pharmaceutical composition contains about 0.1 to 10 mg, for example, 0.5 to 10 mg, of (R)-Compound 1 and a pharmaceutically acceptable excipient. In other embodiments, the pharmaceutical composition comprises lactose anhydrous, microcrystalline cellulose, croscarmellose sodium, colloidal silicon dioxide, and magnesium stearate.

The amount of Compound 1 or (R)-Compound 1 used alone or in the pharmaceutical formulations may also be expressed by way of an amount. In some embodiments, the pharmaceutical formulations contain about 0.1 to about 10 mg of Compound 1 or (R)-Compound 1, e.g., about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of Compound 1 or (R)-Compound 1. In other embodiments, the pharmaceutical formulations contain about 0.5 mg of Compound 1 or (R)-Compound 1. In further embodiments, the pharmaceutical formulations contain about 1 mg of Compound 1 or (R)-Compound 1. In yet other embodiments, the pharmaceutical formulations contain about 2 mg of Compound 1 or (R)-Compound 1. In still further embodiments, the pharmaceutical formulations contain about 3 mg of Compound 1 or (R)-Compound 1. In yet other embodiments, the pharmaceutical formulations contain about 4 mg of Compound 1 or (R)-Compound 1. In still further embodiments, the pharmaceutical formulations contain about 5 mg of Compound 1 or (R)-Compound 1. In other embodiments, the pharmaceutical formulations contain about 6 mg of Compound 1 or (R)-Compound 1. In further embodiments, the pharmaceutical formulations contain about 7 mg of Compound 1 or (R)-Compound 1. In yet other embodiments, the pharmaceutical formulations contain about 8 mg of Compound 1 or (R)-Compound 1. In still further embodiments, the pharmaceutical formulations contain about 9 mg of Compound 1 or (R)-Compound 1. In other embodiments, the pharmaceutical formulations contain about 10 mg of Compound 1 or (R)-Compound 1.

When used alone, about 0.1 to about 10 mg of Compound 1 or (R)-Compound 1, e.g., about 0.1, about 0.2, about 0.3, about 0.4, about 0.5, about 0.6, about 0.7, about 0.8, about 0.9, about 1, about 1.5, about 2, about 2.5, about 3, about 3.5, about 4, about 4.5, about 5 mg, about 5.5 mg, about 6 mg, about 6.5 mg, about 7 mg, about 7.5 mg, about 8 mg, about 8.5 mg, about 9 mg, about 9.5 mg, or about 10 mg may be administered to the human. In other embodiments, about 0.5 mg of Compound 1 or (R)-Compound 1 is administered to the human. In further embodiments, about 1 mg of Compound 1 or (R)-Compound 1 is administered to the human. In yet other embodiments, about 2 mg of Compound 1 or (R)-Compound 1 is administered to the human. In still further embodiments, about 3 mg of Compound 1 or (R)-Compound 1 is administered to the human. In other embodiments, about 4 mg of Compound 1 or (R)-Compound 1 is administered to the human. In further embodiments, about 5 mg of Compound 1 or (R)-Compound 1 is administered to the human. In yet other embodiments, about 6 mg of Compound 1 or (R)-Compound 1 is administered to the human. In still further embodiments, about 7 mg of Compound 1 or (R)-Compound 1 is administered to the human. In other embodiments, about 8 mg of Compound 1 or (R)-Compound 1 is administered to the human. In further embodiments, about 9 mg of Compound 1 or (R)-Compound 1 is administered to the human. In yet other embodiments, about 10 mg of Compound 1 or (R)-Compound 1 is administered to the human.

Compound 1 or (R)-Compound 1, or a pharmaceutical composition containing Compound 1 or (R)-Compound 1, may be administered on an hourly, daily, or weekly basis. Desirably, Compound 1 or (R)-Compound 1, or a pharmaceutical composition containing Compound 1 or (R)-Compound 1, is administered on a daily basis. In some embodiments, about 0.1 to about 30 mg/day of Compound 1 or (R)-Compound 1 is administered. In some embodiments, about 0.1 to about 5 mg/day of Compound 1 or (R)-Compound 1, e.g., about 0.1 mg/day, about 0.2 mg/day, about 0.3 mg/day, about 0.4 mg/day, about 0.5 mg/day, about 0.6 mg/day, about 0.7 mg/day, about 0.8 mg/day, about 0.9 mg/day, about 1 mg/day, about 1.5 mg/day, about 2 mg/day, about 2.5 mg/day, about 3 mg/day, about 3.5 mg/day, about 4 mg/day, about 4.5 mg/day, about 5 mg/day, about 5.5 mg/day, about 6 mg/day, about 6.5 mg/day, about 7 mg/day, about 7.5 mg/day, or about 8 mg/day is administered to the human. In other embodiments, about 0.5 mg/day of Compound 1 or (R)-Compound 1 is administered to the human. In further embodiments, about 1 mg/day of Compound 1 or (R)-Compound 1 is administered to the human. In yet other embodiments, about 2 mg/day of Compound 1 or (R)-Compound 1 is administered to the human. In still further embodiments, about 3 mg/day of Compound 1 or (R)-Compound 1 is administered to the human. In other embodiments, about 4 mg/day of Compound 1 or (R)-Compound 1 is administered to the human. In further embodiments, about 5 mg/day of Compound 1 or (R)-Compound 1 is administered to the human. In yet other embodiments, about 6 mg/day of Compound 1 or (R)-Compound 1 is administered to the human. In still further embodiments, about 7 mg/day of Compound 1 or (R)-Compound 1 is administered to the human. In other embodiments, about 8 mg/day of Compound 1 or (R)-Compound 1 is administered to the human. In further embodiments, about 9 mg/day of Compound 1 or (R)-Compound 1 is administered to the human. In yet other embodiments, about 10 mg/day of Compound 1 or (R)-Compound 1 is administered to the human.

Compound 1 or (R)-Compound 1 may be administered in a single dose or divided doses. In some embodiments, Compound 1 or (R)-Compound 1 is administered in a single dose. In further embodiments, Compound 1 or (R)-Compound 1 is administered in divided doses. For example, Compound 1 or (R)-Compound 1 is administered in a divided dose, such as in two doses, three doses, or four doses. For example, in some aspects, the patient is dosed 4 mg of Compound 1 or (R)-Compound by administering a total of two 2 mg tablets. In other examples, the patient is dosed 6 mg of Compound 1 or (R)-Compound by administering a total of three 2 mg tablets. In further examples, the patient is dosed 8 mg of Compound 1 or (R)-Compound by administering a total of four 2 mg tablets. One of skill in the art would be able to determine and use other combinations of the tablet doses based on the dosage of Compound 1 or (R)-Compound 1 needed.

Compound 1 or (R)-Compound 1 or pharmaceutical formulations containing the same may be administered by any acceptable route. In some embodiments, administration is oral, transdermal, parenteral, or a combination thereof. In further embodiments, administration is oral.

Compound 1 or (R)-Compound 1, or a pharmaceutical formulation containing Compound 1 or (R)-Compound 1, may be formulated for administration in solid or liquid forms. In some embodiments, Compound 1 or (R)-Compound 1, or a pharmaceutical formulation containing Compound 1 or (R)-Compound 1, is formulated in the form of a tablet, caplet, capsule, powder, softgel, suspension or liquid, or a combination thereof. In other embodiments, Compound 1 or (R)-Compound 1, or a pharmaceutical formulation containing Compound 1 or (R)-Compound 1, is formulated in the form of a tablet. In further embodiments, Compound 1 or (R)-Compound 1, or a pharmaceutical formulation containing Compound 1 or (R)-Compound 1, is formulated in the form of a caplet. In yet other embodiments, Compound 1 or (R)-Compound 1, or a pharmaceutical formulation containing Compound 1 or (R)-Compound 1, is formulated in the form of a capsule. In still further embodiments, each tablet, caplet, capsule, powder, softgel, suspension or liquid dose contains about 0.5 to about 5 mg, i.e., about 0.5 mg, about 1 mg, about 1.5 mg, about 2 mg, about 2.5 mg, about 3 mg, about 3.5 mg, about 4 mg, about 4.5 mg, or about 5 mg of Compound 1 or (R)-Compound 1. In other embodiments, each tablet, caplet, capsule, powder, softgel, suspension or liquid dose contains about 0.5 mg of Compound 1 or (R)-Compound 1. In further embodiments, each tablet, caplet, capsule, powder, softgel, suspension or liquid dose contains about 1 mg of Compound 1 or (R)-Compound 1. In yet other embodiments, each tablet, caplet, capsule, powder, softgel, suspension or liquid dose contains about 2 mg of Compound 1 or (R)-Compound 1. In still further embodiments, each tablet, caplet, capsule, powder, softgel, suspension or liquid dose contains about 2 mg of Compound 1 or (R)-Compound 1. In other embodiments, each tablet, caplet, capsule, powder, softgel, suspension or liquid dose contains about 3 mg of Compound 1 or (R)-Compound 1. In further embodiments, each tablet, caplet, capsule, powder, softgel, suspension or liquid dose contains about 4 mg of Compound 1 or (R)-Compound 1. In yet other embodiments, each tablet, caplet, capsule, powder, softgel, suspension or liquid dose contains about 5 mg of Compound 1 or (R)-Compound 1.

In certain aspects, the solid or liquid form contains the patient's dose of Compound 1 or (R)-Compound 1. In other embodiments, it may be necessary to administer two or more doses of the solid or liquid form, i.e., divided doses, to the patient in order to achieve the desired dosage for the patient.

Compound 1 or (R)-Compound 1 described herein is useful in inhibiting aldosterone synthase. Compound 1 or (R)-Compound 1 is useful in a variety of treatment methods. In some embodiments, the disclosure provides methods of treating hypertension using Compound 1 or (R)-Compound 1. In further embodiments, the disclosure provides methods of treating primary aldosteronism using Compound 1 or (R)-Compound 1. In further embodiments, the disclosure provides methods of treating CKD using Compound 1 or (R)-Compound 1. The methods include administering to the patient Compound 1 or (R)-Compound 1 or a pharmaceutical formulation comprising Compound 1 or (R)-Compound 1.

After treatment with Compound 1 or (R)-Compound 1, the patient's seated blood pressure (BP) is lowered to approximately <130/80 mmHg, such as after about 1, about 2, about 3, about 4, about 5, about 6, about 7, about 8, about 9, about 10, about 11, or about 12 weeks of treatment. For example, in some embodiments, administration of Compound 1 or (R)-Compound 1 results in a mean decrease from baseline in SBP by about 1, about 2, about 3, about 4, about 5, about 6, about 7, about 8, about 9, about 10, about 11, about 12, about 13, about 14, about 15, about 16, about 17, about 18, 19, about 20, about 21, about 22, about 23, about 24, 25, about 26, about 27, about 28, about 29, or about 30 mmHg. In some embodiments, administration of Compound 1 or (R)-Compound 1 results in a mean decrease from baseline in seated systolic blood pressure (SBP) after about 1, about 2, about 3, about 4, about 5, about 6, about 7, about 8, about 9, about 10, about 11, or about 12 weeks of treatment. For example, administration of Compound 1 or (R)-Compound 1 results in a mean decrease from baseline in seated SBP after about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 4 to about 11, about 4 to about 10, about 4 to about 9, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks. In other embodiments, administration of Compound 1 or (R)-Compound 1 results in a mean decrease in baseline in seated diastolic blood pressure (DBP) after about 12 weeks of treatment. For example, in some embodiments, administration of Compound 1 or (R)-Compound 1 results in a mean decrease from baseline in DBP by about 1, about 2, about 3, 4 about, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mmHg. In further embodiments, administration of Compound 1 or (R)-Compound 1 results in a mean decrease from baseline in seated SBP and a mean decrease in baseline in seated DBP after about 1, about 2, about 3, about 4, about 5, about 6, about 7, about 8, about 9, about 10, about 11, or about 12 weeks of treatment. For example, administration of Compound 1 or (R)-Compound 1 results in a mean decrease from baseline in seated DBP after about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 4 to about 11, about 4 to about 10, about 4 to about 9, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks.

After treatment with Compound 1 or (R)-Compound 1, the patient's aldosterone levels, renin levels, or a combination thereof is lowered, such as after about 1, about 2, about 3, about 4, about 5, about 6, about 7, about 8, about 9, about 10, about 11, or about 12 weeks of treatment. For example, in some embodiments, administration of Compound 1 or (R)-Compound 1 results in a mean decrease from baseline in SBP by about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mmHg. In some embodiments, administration of Compound 1 or (R)-Compound 1 results in a mean decrease from baseline in seated systolic blood pressure (SBP) after about 1, about 2, about 3, about 4, about 5, about 6, about 7, about 8, about 9, about 10, about 11, or about 12 weeks of treatment. For example, administration of Compound 1 or (R)-Compound 1 results in a mean decrease from baseline in seated SBP after about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 12, about 3 to about 11, about 3 to about 10, about 3 to about 9, about 3 to about 8, about 3 to about 7, about 3 to about 6, about 3 to about 5, about 3 to about 4, about 4 to about 12, about 4 to about 11, about 4 to about 10, about 4 to about 9, about 4 to about 8, about 4 to about 7, about 4 to about 6, about 4 to about 5, about 5 to about 12, about 5 to about 11, about 5 to about 10, about 5 to about 9, about 5 to about 8, about 5 to about 7, about 5 to about 6, about 6 to about 12, about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks. In certain In other embodiments,

In some embodiments, the patient does not respond to one or more stable background hypertensive regimen. A “stable background hypertensive regimen” includes any regimen that lowers the patient's blood pressure. The regimen may include performing one or more therapies such as daily activities or taking one or more antihypertensive agents. In some embodiments, the stable background hypertensive regimen is one or more daily activities. Examples of daily activities that may be used to treat hypertension or primary aldosteronism include, without limitation, healthy eating, lowering salt intake, getting regular physical activity, maintaining a healthy weight, losing weight if advised by a physician, and limiting alcohol consumption. In other embodiments, the stable background hypertensive regimen is an antihypertensive agent.

The term “antihypertensive agents” as used herein refers to a medication that lowers a patient's blood pressure. In some embodiments, the antihypertensive agent is a diuretic, loop diuretic, beta-blocker, ACE inhibitor, angiotensin II receptor blocker, calcium channel blocker, alpha blocker, alpha-2 receptor agonist, combined alpha and beta-blocker, central agonists, peripheral adrenergic inhibitor, blood vessel dilator (vasodilator), or combination thereof. In some embodiments, the antihypertensive agent is a diuretic such as a thiazide diuretic, potassium-sparing diuretic, loop diuretic, or a combination diuretic. Examples of thiazide diuretics include chlorthalidone (Hygroton), chlorothiazide (Diuril), hydrochlorothiazide (Esidrix, Hydrodiuril, Microzide), indapamide (Lozol), or metolazone (Mykrox, Zaroxolyn). Examples of potassium-sparing diuretics include amiloride hydrochloride (Midamar), spironolactone (Aldactone), eplerenone (Inspra), or triamterene (Dyrenium). Examples of loop diuretics include furosemide (Lasix) or bumetanide (Bumex). Examples of the combination diuretic include amiloride hydrochloride+hydrochlorothiazide (Moduretic), spironolactone+hydrochlorothiazide (Aldactazide), or triamterene+hydrochlorothiazide (Dyazide, Maxzide). In other embodiments, the antihypertensive agent is a beta-blocker. Examples of beta-blockers include acebutolol (Sectral), atenolol (Tenormin), betaxolol (Kerlone), bisoprolol fumarate (Zebeta), carteolol hydrochloride (Cartrol), metoprolol tartrate (Lopressor), metoprolol succinate (Toprol-XL), nadolol (Corgard), penbutolol sulfate (Levatol), pindolol (Visken), propranolol hydrochloride (Inderal), solotol hydrochloride (Betapace), or timolol maleate (Blocadren). In further embodiments, the antihypertensive agent is a combination beta-blocker/diuretic. An example of the beta-blocker/diuretic combination is hydrochlorothiazide+bisoprolol (Ziac). In yet other embodiments, the antihypertensive agent is an ACE inhibitor. Examples of ACE inhibitors include benazepril hydrochloride (Lotensin), captopril (Capoten), enalapril maleate (Vasotec), fosinopril sodium (Monopril), lisinopril (Prinivel, Zestril), moexipril (Univasc), perindopril (Aceon), quinapril hydrochloride (Accupril), ramipril (Altace), or trandolapril (Mavik). In still further embodiments, the antihypertensive agent is an angiotensin II receptor blocker. Examples of angiotensin II receptor blockers include candesartan (Atacand), eprosartan mesylate (Teveten), irbesartan (Avapro), losartan potassium (Cozaar), telmisartan (Micardis), or valsartan (Diovan). In other embodiments, the antihypertensive agent is a calcium channel blocker. Examples of calcium channel blockers include amlodipine besylate (Norvasc, Lotrel), bepridil (Vasocor), diltiazem hydrochloride (Cardizem CD, Cardizem SR, Dilacor XR, Tiazac), felodipine (Plendil), isradipine (DynaCirc, DynaCirc CR), nicardipine (Cardene SR), nifedipine (Adalat CC, Procardia XL), nisoldipine (Sular), or verapamil hydrochloride (Calan SR, Covera HS, Isoptin SR, Verelan). In further embodiments, the antihypertensive agent is an alpha blocker. Examples of alpha blockers include doxazosin mesylate (Cardura), prazosin hydrochloride (Minipress), or terazosin hydrochloride (Hytrin). In still other embodiments, the antihypertensive agent is an alpha-2 receptor agonist. An example of an alpha-2 receptor agonist is methyldopa. In yet further embodiments, the antihypertensive agent is a combined alpha and beta-blocker. Examples of combined alpha and beta-blockers include carvedilol (Coreg) or labetalol hydrochloride (Normodyne, Trandate). In other embodiments, the antihypertensive agent is a central agonist. Examples of central agonists include alpha methyldopa (Aldomet), clonidine hydrochloride (Catapres), guanabenz acetate (Wytensin), or guanfacine hydrochloride (Tenex). In further embodiments, the antihypertensive agent is a peripheral adrenergic inhibitor. Examples of peripheral adrenergic inhibitors include guanadrel (Hylorel), guanethidine monosulfate (Ismelin), or reserpine (Serpasil). In yet other embodiment, the antihypertensive agent is a blood vessel dilator, i.e., vasodilator. Examples of blood vessel dilators include hydralazine hydrochloride (Apresoline), or minoxidil (Loniten).

In some embodiments, the patient's hypertension or primary aldosteronism does not respond to one or more stable background hypertensive regimen prior to administering Compound 1 or (R)-Compound 1. In other embodiments, the patient's hypertension or primary aldosteronism does not respond to two stable background hypertensive regimens prior to administering Compound 1 or (R)-Compound 1. In further embodiments, the patient's hypertension or primary aldosteronism does not respond to three stable background hypertensive regimens prior to administering Compound 1 or (R)-Compound 1. In other embodiments, the patient's hypertension or primary aldosteronism does not respond to three or more stable background hypertensive regimens prior to administering Compound 1 or (R)-Compound 1.

The disclosure also provides methods for treating hypertension or primary aldosteronism where administration of Compound 1 or (R)-Compound 1 does not result in a clinically significant adverse event in the human. The term “clinically significant” as used herein refers to a result that affects the patient to warrant follow-up with a physician.

The following Examples are provided to illustrate some concepts described within this disclosure. While the Example is considered to provide specific individual embodiments of formulations, methods of preparation and use, the Examples should not be considered to limit the more general embodiments described herein.

In the following Examples, efforts have been made to ensure accuracy with respect to numbers used (e.g., amounts, temperature, etc.) but some experimental error and deviation should be accounted for.

### List of Abbreviations and Definition of Terms

| Abbreviation | Definition |
|---|---|
| ACEi | Angiotensin-converting enzyme inhibitor |
| ACTH | Adrenocorticotropic hormone |
| AE | Adverse event |
| AESI | Adverse event of special interest |
| AOBPM | Automated office blood pressure monitoring |
| API | Active pharmaceutical ingredient |
| ARB | Angiotensin receptor blocker |
| ARR | Aldosterone/plasma renin activity ratio |
| ASI | Aldosterone synthase inhibitor |
| AUC | Area under the concentration time curve |
| AUC₀₋∞ | Area under the concentration-time curve from time 0 extrapolated |
| | to infinity |
| AUC₀₋ₜₗₐₛₜ | Area under the concentration-time curve from time 0 to the time |
| | of the last quantifiable concentration |
| BMI | Body mass index |
| BNP | B-type natriuretic peptide |
| BP | Blood pressure |
| bpm | Beats per minute |
| CABG | Coronary artery bypass graft |
| CCB | Calcium channel blocker |
| CI | Confidence interval |
| CKD | Chronic kidney disease |
| CKD-EPI | Chronic Kidney Disease Epidemiology |
| CL_{R} | Renal clearance |
| Cₘₐₓ | Maximum plasma concentration |
| CV | Cardiovascular |
| CYP | Cytochrome P450 |
| DBP | Diastolic blood pressure |
| DDI | Drug-drug interaction |
| ECG | Electrocardiogram |
| eGFR | Estimated glomerular filtration rate |
| EOT | End of Treatment |
| ET | Early Termination |
| FSH | Follicle-stimulating hormone |
| GDF-15 | Growth differentiation factor-15 |
| GFR | Glomerular filtration rate |
| GMP | Good manufacturing practice |
| HBA1c | Glycosylated hemoglobin |
| HBsAg | Hepatitis B surface antigen |
| HCV | Hepatitis C virus |
| HIV | Human immunodeficiency disease |
| HTN | Hypertension |
| IRT | Interactive Response Technology |
| ITT | Intent-to-Treat |
| IV | Intravenous |
| KIM-1 | Kidney injury molecule-1 |
| LLOQ | Lower limit of quantification |
| MAD | Multiple-ascending dose |
| MATE | Multidrug and toxin extrusion |
| MCP1 | Monocyte chemoattractant protein-1 |
| MedDRA | Medical Dictionary for Regulatory Activities |
| mITT | Modified Intent-to-Treat |
| MMRM | Mixed model repeated measures |
| MR | Mineralocorticoid receptor |
| MRA | Mineralocorticoid receptor agonist |
| MTD | Maximum tolerated dose |
| NGAL | Neutrophil gelatinase-associated lipocalin |
| NSAID | Nonsteroidal anti-inflammatory drug |
| OLE | Open label extension |
| PA | Primary aldosteronism |
| PAC | Plasma aldosterone concentration |
| PCI | Percutaneous coronary intervention |
| PD | Pharmacodynamic(s) |
| PGx | Pharmacogenomic(s) |
| PK | Pharmacokinetic(s) |
| POC | Point-of-care or Point-of-contact |
| PP | Per-Protocol |
| PRA | Plasma renin activity |
| QD | Once daily |
| QTc | Corrected QT interval |
| QTcB | QT interval corrected using Fridericia's formula |
| QTcF | Heart rate-corrected QT interval using Frederica's formula |
| RAAS | Renin-angiotensin-aldosterone system |
| rHTN | Treatment-resistant hypertension |
| RNA | Ribonucleic acid |
| SAD | Single-ascending dose |
| SAE | Serious adverse event |
| SAP | Statistical Analysis Plan |
| SBP | Systolic blood pressure |
| SB-RI | Single Blind-Run In |
| SD | Standard deviation |
| SGLT2 | Sodium-glucose cotransporter 2 |
| t_{1/2} | Apparent terminal elimination half-life |
| TEAE | Treatment-emergent adverse event |
| TESAE | Treatment-emergent serious adverse event |
| TGF-β | Transforming growth factor beta |
| Tₘₐₓ | Median time to maximum plasma concentration |
| UACR | Urinary albumin-to-creatinine ratio |
| ULN | Upper limit of normal |

### Example 1

A Phase 1, open-label study of the absorption, metabolism, and excretion of [14C]—(R)-compound 1 following a single oral dose in healthy male subjects.

| Objectives | Endpoints |
|---|---|
| to determine the routes, rates of elimination, | total radioactivity recovery (fet1-t2) in |
| and mass balance of total radioactivity from | urine and feces |
| [14C]-(R)-compound 1 | PK parameters including, but not limited to, |
| to characterize the PK of (R)-compound 1 | AUC₀₋∞, AUC₀₋ₜₗₐₛₜ, Cₘₐₓ, tₘₐₓ, and t_{1/2} for |
| and its primary metabolite following | (R)-compound 1 and metabolite in plasma |
| administration of [14C]-(R)-compound 1 to | cumulative amount of (R)-compound 1 and |
| healthy male subjects | metabolite excreted in urine (Ae), CLR of |
| to characterize the total radioactivity | (R)-compound 1 and metabolite, fraction of |
| following administration of [14C]-(R)- | dose excreted renally ((R)-compound 1 only; |
| compound 1 to healthy subjects | fet1-t2) |
| | PK parameters including, but not limited to, |
| | AUC₀₋∞, AUC₀₋ₜₗₐₛₜ, Cₘₐₓ, tₘₐₓ, and t_{1/2} for |
| | total radioactivity in plasma and whole blood |
| to determine, where possible, the | quantitative metabolic profiles of |
| quantitative metabolite profiles in plasma, | compound 1 in plasma and excreta |
| urine, and feces after [14C]-(R)-compound 1 | identification of (R)-compound 1 |
| to determine, where possible, the chemical | metabolites in plasma and excreta |
| structure of major metabolites in plasma, | incidence and severity of AEs |
| urine, and feces after [14C]-(R)-compound 1 | incidence of laboratory abnormalities, |
| to assess the safety and tolerability of | based on hematology, clinical chemistry, and |
| [14C]-compound 1 when administered to | urinalysis test results |
| healthy subjects | 12-lead ECG parameters |
| | vital signs measurements |
| | physical examinations |

Potential subjects will be screened to assess their eligibility to enter the study within 28 days prior to the dose administration. Subjects will be admitted into the study site on Day 1 and be confined to the study site until at least Day 9. On the morning of Day 1, all subjects will receive a single oral dose of [14C]—(R)-compound. Subjects will be discharged if the following discharge criteria are met: plasma radioactivity levels below the limit of quantitation for 2 consecutive collections, ≥90% mass balance recovery, and ≤1% of the total radioactive dose is recovered in combined excreta (urine and feces) in 2 consecutive 24-hour periods. If discharge criteria are not met by Day 9, subjects will remain in the study site until all discharge criteria are met up to a maximum of Day 15 to continue 24-hour blood, urine, and feces collections, unless otherwise agreed upon by the sponsor and investigator. The study site will contact subjects through a phone call 3 days (±1 day) after discharge from the study site.

### Diagnosis and Criteria for Inclusion

Healthy male subjects aged between 18 and 55 years (inclusive) with a body mass index between 18.0 and 32.0 kg/m² (inclusive).

Subjects must satisfy all of the following criteria at the screening visit, unless otherwise stated:
1. Males of any race between 18 and 55 years of age, inclusive.
2. Body mass index between 18.0 and 32.0 kg/m², inclusive.
3. In good health, determined by no clinically significant findings from medical history, 12-lead ECG, vital signs measurements (seated and orthostatic), and clinical laboratory evaluations (congenital nonhemolytic hyperbilirubinemia [e.g., suspicion of Gilbert's syndrome based on total and direct bilirubin] is not acceptable) at screening and/or check-in and from the physical examination at check-in, as assessed by the investigator (or medically qualified designee).
4. Normal renal function, defined as estimated GFR≥70 mL/min/1.73 m² at screening and check-in calculated using Chronic Kidney Disease Epidemiology Collaboration method.
5. Subjects will agree to use contraception.
6. Able to comprehend and willing to sign an ICF and to abide by the study restrictions.
7. History of a minimum of 1 bowel movement per day.
8. Subjects are required to refrain from donation of sperm from check-in until 90 days after discharge.

Subjects are excluded from the study if they satisfy any of the following criteria at the screening visit, unless otherwise stated:

Medical conditions
1. Significant history or clinical manifestation of any autoimmune, immunologic, metabolic, allergic, dermatological, hepatic, renal, hematological, pulmonary, cardiovascular, gastrointestinal, neurological (including seizure), neuromuscular, musculoskeletal, respiratory, endocrine, or psychiatric disorder, cancer (with the exception of basal or squamous cell carcinoma of the skin and cancer that resolved or has been in remission for >5 years prior to the screening visit), as determined by the investigator (or medically qualified designee).
2. A personal or family history of long QT syndrome, Torsades de Pointes, other complex ventricular arrhythmias, or family history of sudden death.
3. History of, or current, clinically significant arrhythmias, as judged by the investigator (or medically qualified designee), including ventricular tachycardia, ventricular fibrillation, atrial fibrillation, sinus node dysfunction, or clinically significant heart block. Subjects with minor forms of ectopy (e.g., premature atrial contractions) are not necessarily excluded and may be discussed with the medical monitor for inclusion.
4. Prolonged QTcF (>450 msec).
5. History of significant hypersensitivity, intolerance, or allergy to any drug compound, food, or other substance, unless approved by the investigator (or medically qualified designee).
6. Surgical conditions or any condition that in the opinion of investigator (or medically qualified designee) would potentially alter absorption, distribution, metabolism, and/or excretion of orally administered drugs (uncomplicated appendectomy and hernia repair will be allowed, cholecystectomy and gastric bypass surgery prohibited).
7. Confirmed (e.g., 2 consecutive measurements) systolic BP>140 or <90 mmHg, diastolic BP>90 or <50 mmHg, and pulse rate >100 or <45 beats per minute (bpm).
8. Postural tachycardia (i.e., >30 bpm upon standing) or orthostatic hypotension (i.e., a fall in systolic BP of ≥20 mmHg or diastolic BP of ≥10 mmHg upon standing).
9. Serum potassium >upper limit of normal (5.3 mmol/L; ULN) of the reference range and serum sodium <lower limit of normal (135 mmol/L) of the reference range (confirm by repeat).
10. Aspartate aminotransferase, alanine aminotransferase, or total bilirubin values >1.2×ULN.
11. Any other clinical laboratory values which are meaningfully outside of normal limits (based on laboratory normal range) in the opinion of the investigator (or medically qualified designee).
12. A known history of porphyria, myopathy, or active liver disease.
13. Positive hepatitis panel and/or positive human immunodeficiency virus test.
14. Positive COVID-19 test at screening or check-in.

### Prior/Concomitant Therapy

15. Administration of a COVID-19 vaccine in the past 30 days prior to dosing.
16. Use of any prescription medications, including topicals or over-the-counter medications (other than occasional use of acetaminophen or nonsteroidal anti-inflammatory drugs, such as ibuprofen or naproxen, according to the package insert); herbal supplements; dietary supplements; or nutraceuticals within 14 days prior to dosing or 5 half-lives, whichever is longer, or an unwillingness to refrain from these medications through discharge from the study site. Use of over-the-counter topical medications may be permitted in consultation with the sponsor. In addition, use of medications for which 5 half-lives exceeds 14 days must be discussed with and approved by the sponsor prior to subject enrollment.
17. Corticosteroid use (systemic or extensive topical use) within 3 months (90 days) prior to dosing.

### Investigational Medicinal Products, Dose, and Mode of Administration

Single oral dose of 10 mg of [14C]—(R)-compound 1 containing approximately 100 μCi as a capsule after an overnight fast.

### Duration of Subject Participation in the Study

Planned screening duration: approximately 4 weeks.

Planned study duration (screening to follow-up phone call): maximum of approximately 7 weeks.

### Description, Storage, Packaging, and Labeling

Active pharmaceutical ingredient (API; radiolabeled) will be supplied as blended hot/cold [14C]—(R)-compound 1 by the sponsor (or designee) along with the batch/lot number and certificate of analysis. The provided blend will have been fully tested for purity (radiochemical and ultraviolet) and all specifications are required to be met prior to release.

Each subject dose contains a total of 10 mg containing approximately 100 μCi of [14C]—(R)-compound 1 (may be administered as multiple capsules). The completed drug product will be released by a good manufacturing practice (GMP) quality auditor under GMP conditions prior to administration to subjects.

### Study Treatment Administration

Each dose of [14C]—(R)-compound 1 will be administered orally with 240 mL of room temperature water. All subjects will fast overnight (at least 10 hours) and will refrain from consuming water for 1 hour prior to dosing. Subjects will refrain from consuming water until 2 hours postdose, excluding the amount of water consumed at dosing, and will fast until approximately 4 hours postdose. At all other times during the study, subjects may consume water ad libitum.

Subjects will be dosed in numerical order while seated and will not be permitted to lie supine for 4 hours after administration of [14C]—(R)-compound 1, except as necessitated by the occurrence of an AE(s) and/or study procedures.

Subjects will be observed during the first 4 hours postdose and will be escorted to the restroom, as required, during this time.

### Pharmacokinetic Analyses

The mass balance recovery of total radioactivity (percentage of the dose administered recovered in urine, feces, and total excreta) will be calculated by the radioanalysis laboratory. Pharmacokinetic parameters will be determined from individual (non-pooled) plasma and urine concentrations of (R)-compound 1 and metabolite, and plasma and whole blood total radioactivity concentrations using standard noncompartmental methods. Full details of PK parameters will be presented in the statistical analysis plan for this study.

### Example 2

A SAD study involved single oral doses of (R)-compound 1 up to 360 mg, which were well tolerated by healthy subjects. There were no deaths, serious adverse events (SAEs), or dose-limiting events and the maximum tolerated dose observed was at the highest dose tested (360 mg). Overall, the most frequently reported AEs following administration of a single dose of (R)-compound 1 were headache, nasopharyngitis, diarrhea, asthenia, dizziness, and nausea.

Mild, dose-dependent decreases in plasma sodium levels and increases in plasma potassium levels were observed with corresponding changes in urine sodium and potassium levels. Of note, despite an initial increase in the urine sodium:potassium ratio, indicating that the sodium loss in urine is greater than the potassium retention, the ratio normalized by Day 10, suggesting that the balance between sodium excretion and potassium absorption was restored.

The change in the ratio appears to be mediated by a greater elimination of sodium in the urine on Day 1 as compared to sodium elimination in the urine on Day 10 as potassium appears not to change over the course of the 10-day treatment period.

Increases in blood urea nitrogen and creatinine were observed following administration of (R)-compound 1 along with a mild reduction (<15%) in glomerular filtration rate (GFR). The presence of an increased blood urea nitrogen:creatinine ratio with reduced GFR suggests that (R)-compound 1 is producing a mild diuretic effect. Finally, subjects receiving (R)-compound 1 experienced more pronounced decreases in body weight and body mass index as compared to subjects receiving placebo, in all likelihood due to the previously noted mild diuretic effects. Values returned to normal by the time of the follow-up visit.

The SAD study indicates that following oral administration, (R)-compound 1 was rapidly absorbed with a median time (tmax) to maximum observed concentration (Cₘₐₓ) between 0.5 and 2 hours. A second, generally lower peak was often observed at 3 to 4 hours postdose. Thereafter, concentrations declined from peak in a biphasic manner with a long median terminal elimination half-life of approximately 25 to 31 hours. Over the dose range (through 10 mg), peak and overall exposures (as assessed by Cₘₐₓ and AUC from time 0 to infinity [AUC_{0-inf}]) increased in a generally dose proportional manner. Approximately 110% of the dose is recovered unchanged in the urine.

Single doses of (R)-compound 1 reduced plasma and urine aldosterone levels in a dose-dependent manner.

After oral dosing, an average 10.8% (range of individual values from 4.97% to 22.5% across all dose groups) of the dose was recovered unchanged ((R)-compound 1) in the urine collected up to 48 hours postdose while an average 0.22% (0.039% to 0.599% across all dose groups) of the dose was recovered as active metabolite in the urine.

The observed CLR (ranging from 278 to 443 mL/h, depending on dose) was less than the product of unbound fraction in plasma and the GFR in healthy subjects (fu*GFR=0.26*7200 mL/h=1870 mL/h), indicating the presence of a net tubular reabsorption. Renal clearance represented on average 15.5% (range of individual values from active metabolite in the urine 6.53% to 31.8% across all dose groups) of the total plasma clearance.

Following oral administration, (R)-compound 1 was rapidly absorbed with a median time to Cₘₐₓ(Tₘₐₓ) typically observed between 0.5 and 2 hours. A second, generally lower peak was often observed at 3 to 4 hours post-dose. Thereafter, concentrations declined from peak in a biphasic manner with a long median terminal elimination half-life of approximately 25 to 31 hours. Over the anticipated therapeutically relevant dose range (through 10 mg), peak and overall exposures (as assessed by Cₘₐₓ and area under the concentration-time curve [AUC]) increased in a generally dose-proportional manner. Approximately 110% of the dose was recovered unchanged in the urine.

Single oral doses of up to 360 mg (R)-compound 1 did not affect serum electrolyte (chloride, potassium, sodium, and phosphate) levels, with no difference for subjects on active treatment versus those on placebo. Urine sodium and the sodium to potassium ratio both increased, with the sodium loss in urine greater than the potassium retention. No change in urine creatinine was apparent.

Single doses of (R)-compound 1 reduced plasma and urine aldosterone levels by approximately 85% to 90% in a dose-dependent manner, consistently reaching a maximum effect at a dose of 10 mg (R)-compound 1 under the different conditions tested (Cortrosyn® challenge, standing, normal salt diet, and low salt diet conditions). No change in plasma cortisol levels was apparent across the full dose range tested (0 to 360 mg (R)-compound 1).

A maximum effect on aldosterone reduction was consistently achieved at a dose of 10 mg under the different conditions tested in healthy subjects (Cortrosyn® challenge, standing, normal salt diet, and low salt diet conditions). No change in plasma cortisol levels after the Cortrosyn challenge was apparent across the full dose range tested (0 to 360 mg of (R)-compound 1). Although there was no effect on cortisol levels through 360 mg, some partial inhibition of the CYP11B1 enzyme at exposures well above those considered to be therapeutically relevant may occur based on observed increases in 11-deoxycortisol (at doses of 180 mg and 360 mg) and 11-DOC (at doses ≥90 mg).

### Example 3

A Double-blind, placebo-controlled, multicenter study to evaluate the efficacy and safety of multiple dose strengths of (R)-compound 1 as compared to placebo after 8 weeks of treatment in patients with uncontrolled hypertension receiving 1 antihypertensive agent.

### Indication

Reduction of systolic blood pressure (SBP) in patients with hypertension (HTN).

### Objectives

One objective is to demonstrate that at least 1 dose strength of (R)-compound 1 is superior to placebo for the change from baseline in mean seated SBP after 8 weeks of treatment in patients with uncontrolled HTN who have higher serum aldosterone levels and are receiving 1 background antihypertensive agent (Part 1).

Other objectives are to evaluate the following parameters in the study population of individuals with uncontrolled hypertension who have higher serum aldosterone levels and are receiving 1 background anti-hypertensive agent:
The change from baseline in mean seated diastolic blood pressure (DBP) with each of the selected dose strengths of (R)-compound 1 compared to placebo after 8 weeks of treatment (Part 1).
The change from baseline in 24-hour urine aldosterone and serum aldosterone levels with each of the selected dose strengths of (R)-compound 1 compared to placebo after 8 weeks of treatment (Part 1); and
The percentage of patients achieving a mean seated SBP <130 mmHg (“responders”) with each of the selected dose strengths of (R)-compound 1 compared to placebo after 8 weeks (Part 1).

Other objectives are to evaluate the following:
The change in 24-hour urine aldosterone and serum aldosterone levels from values measured at the end of Part 1 to those measured following 4 weeks of treatment with (R)-compound 1 2 mg dose strength and no background antihypertensive agent at the end of Part 2
The percentage of patients maintaining a mean seated SBP <130 mmHg (“responders”) when treated with (R)-compound 1 2 mg dose strength alone and no background antihypertensive agent for 4 weeks during Part 2.

### Safety Objectives

The safety objectives for both Parts 1 and 2 are to evaluate the following:
Vital signs, standing blood pressure (BP) and heart rate, physical examinations, electrocardiogram (ECG), body weight, and clinical laboratory assessments, including standard safety chemistry panel, hematology, coagulation, and urinalysis;
Treatment-emergent adverse events (TEAEs);
Treatment-emergent serious adverse events (TESAEs);
TEAEs leading to premature discontinuation of study drug;
Treatment-emergent marked laboratory abnormalities; and
Change in standing SBP (measured pre-dose at the clinical site) from baseline to End of Treatment (EOT) (Visit 9).

### Pharmacokinetic-Pharmacodynamic Objective

The pharmacokinetic (PK)-pharmacodynamic (PD) objectives for both Parts 1 and 2 are to evaluate the exposure-response relationships of (R)-compound 1 using measures of safety, PD, and/or efficacy.

### Inclusion Criteria

Patients must meet all the following criteria to be eligible to participate in the study:
1. Are adult male and female patients ≥18 years;
2. Are on a stable regimen of a single background antihypertensive agent at the maximum tolerated dose (MTD) (in the opinion of the Investigator) for at least 8 weeks and would be considered a candidate for addition of a second antihypertensive agent at the time of Screening;
Acceptable classes of antihypertensive agents being used as primary treatment for systemic HTN include angiotensin-converting enzyme inhibitors (ACEis)/angiotensin receptor blockers (ARBs), calcium channel blockers, and diuretics (other than mineralocorticoid receptor antagonists [MRAs] or potassium sparing diuretics). Anti-anginal nitrates, including nitroglycerine, isosorbide mononitrate, and isosorbide dinitrate are not considered antihypertensive agents.
Patients not on the maximal dose of an acceptable anti-hypertensive agent should have source documentation provided that explains the investigator's rationale for stipulating that the dose the patient is receiving is that patient's MTD; Medications consisting of two active agents (i.e., ACE inhibitor+diuretic, CCB+ARB, etc.) are not considered single antihypertensive agents.
3. Have a mean seated SBP≥140 mmHg at Screening (Visit 1) and Visit 2; Patients with mean seated SBP≥130 mmHg may be eligible if diabetic.

Mean seated SBP is defined as the average of 3 seated SBP measurements at any single clinical site visit.
4. Have ≥70% and ≤120% adherence to their antihypertensive medication and the (R)-compound 1 placebo during the Run-In Period, based on pill counts on the morning of Visit 2;
5. Have a serum aldosterone ≥7 ng/dL at Screening;
Patients taking an ACEi or ARB may be enrolled with a serum aldosterone ≥6 ng/dL.
6. If taking a sodium-glucose cotransporter 2 (SGLT2) inhibitor at Screening (Visit 1), the regimen must be stable for a period of at least 8 weeks before Visit 2 and be expected to remain at that dose over the study period;
It is expected that patients not currently taking SGLT2 inhibitor(s) will not initiate this class of medication during the entire Treatment Period.
7. Agree to comply with the contraception and reproduction restrictions of the study as follows:
Female patients who are postmenopausal must have had no menstrual bleeding for at least 1 year at Screening and either be >60 years or have an elevated plasma follicle-stimulating hormone level >40 mIU/mL at Screening;
Female patients of childbearing potential (i.e., ovulating, pre-menopausal, and not surgically sterile) must have a documented negative pregnancy test at Screening (Visit 1) and Visit 2;
Female patients of childbearing potential must use a highly effective method of contraception (i.e., <1% failure rate) from Day 1 through 30 days after the last administration of study drug. Acceptable methods of contraception for female patients enrolled in the study include the following:
   Surgical sterilization (tubal ligation);
   Intrauterine device for at least 12 weeks before Screening;
   Hormonal contraception (oral, implant, injection, ring, or patch) for at least 12 weeks before Screening; or
   Diaphragm used in combination with spermicide.
Male patients must agree to abstain from sperm donation from Day 1 through 90 days after the final dose of study drug.
8. Are able and willing to give informed consent for participation in the clinical study.

### Exclusion Criteria

Patients who meet any of the following criteria are excluded from participation in the study:
1. Have a mean seated SBP≥180 mmHg at Screening (Visit 1) or baseline (Visit 2);
2. Have body mass index >50 kg/m² at Screening;
3. Have upper arm circumference that does not meet the cuff measurement criteria for the selected BP machine at Screening;
4. Are using an alpha or beta blocker for the treatment of systemic HTN or another primary condition/indication (e.g., benign prostatic hyperplasia, migraine headache, heart failure);
5. Are not willing or not able to discontinue an MRA or a potassium sparing diuretic as part of an existing antihypertensive regimen;
6. Are not willing to discontinue taking a potassium supplement;
7. Are expected to receive or are receiving any of the exclusionary drugs (strong cytochrome P450 3A inducers and/or chronic use of non-steroidal anti-inflammatory drugs [NSAIDs]); Patients on chronic NSAIDs who are willing to come off at Screening for the course of the study may be allowed to participate.
8. Have known renal artery stenosis, uncontrolled or untreated hyperthyroidism, uncontrolled or untreated hypothyroidism, hyperparathyroidism, pheochromocytoma, Cushing's syndrome, or aortic coarctation; Patients with primary aldosteronism may be considered for enrollment unless an adrenalectomy is expected before the end of their study participation.
9. Have documented estimated glomerular filtration rate <30 mL/min/1.73 m² calculated using the Chronic Kidney Disease Epidemiology Collaboration equation at Screening;
10. Have known and documented New York Heart Association stage III or IV chronic heart failure at Screening;
11. Have had a stroke, transient ischemic attack, hypertensive encephalopathy, acute coronary syndrome, or hospitalization for heart failure within 6 months before Screening;
12. Have known current severe left ventricular outflow obstruction, such as obstructive hypertrophic cardiomyopathy and/or severe aortic valvular disease diagnosed from a prior echocardiogram;
13. Have a planned coronary revascularization (percutaneous coronary intervention [PCI] or coronary artery bypass graft [CABG]) or any major surgical procedure;
14. Have had CABG or other major cardiac surgery (e.g., valve replacement), peripheral arterial bypass surgery, or PCI within 6 months before Screening;
15. Have chronic permanent atrial fibrillation;
16. Have uncontrolled diabetes with glycated hemoglobin >10% at Screening;
17. Have dialysis or kidney transplantation planned during the course of this study;
18. Have had prior solid organ transplant and/or cell transplants;
19. Have known hypersensitivity to (R)-compound 1, drugs of the same class, or any of its excipients;
20. Have any clinically relevant medical or surgical conditions, including unstable conditions and/or conditions treated with systemic immunosuppressants including corticosteroids that, in the opinion of the Investigator, would put the patient at risk by participating in the study;
21. Have evidence of any of the following (1 retest is allowed at Screening): a. White blood cell count >15×10⁹/L or absolute neutrophil count <1×10⁹/L at Screening;
b. Sodium <130 mEq/L at Screening (Visit 1);
   If the Investigator elects to correct the serum sodium level and/or offers to manage the condition, 1 retest for Screening is allowed at least 1 week prior to Visit 2. A repeat serum sodium <130 mEq/L will disqualify a patient from the study.
c. Potassium <3.5 mEq/L at Screening (Visit 1);
d. Potassium >5 mEq/L at Screening (Visit 1);
If the Investigator elects to correct the serum potassium level and/or offers to manage the condition, 1 retest for Screening is allowed at least 1 week prior to Visit 2. A repeat serum potassium >5 mEq/L will disqualify a patient from the study.
e. Hemoglobin <10 g/dL at Screening and/or anticipated initiation of erythropoietin stimulating agents and/or planned transfusion within 2 months after Screening; or
f. Serum aspartate aminotransferase and/or alanine aminotransferase >3×the upper limit of normal range, with a corresponding total bilirubin >2 mg/dL at Screening. If patient has a history of Gilbert's syndrome, bilirubin may be >2 mg/dL at Screening.
22. Are positive for HIV antibody, hepatitis C virus RNA, or hepatitis B surface antigen at Screening;
23. Have typical consumption of ≥14 alcoholic drinks weekly; 1 drink of alcohol is equivalent to 12 pint of beer (285 mL), 1 glass of spirits (25 mL), or 1 glass of wine (125 mL).
24. Are pregnant, breastfeeding, or planning to become pregnant during the study;
25. Have participated in another clinical study involving any investigational drug within 30 days prior to Screening, or plans to participate in another clinical study within 30 days of discontinuation of study drug;
26. Have received experimental therapy with a small molecule within 30 days of Day 1 or 5 half-lives, whichever is greater, or a large molecule within 90 days of Day 1 or 5 half-lives, whichever is greater; or
27. Are unsuitable for any other reason that may either place the patient at increased risk during participation or interfere with the interpretation of the study outcomes by the Investigator, after reviewing medical and psychiatric history, physical examination, and laboratory evaluation.

### Study Design and Duration

This is a Phase 2, randomized, multicenter study to evaluate the efficacy and safety of multiple dose strengths of (R)-compound 1 in the treatment of patients with HTN. To be considered for study participation, patients must have uncontrolled HTN (mean seated SBP≥140 mmHg [or ≥130 mmHg if diabetic]) despite being on a stable regimen of a single background antihypertensive agent at the MTD (in the opinion of the Investigator) for at least 8 weeks and would be considered a candidate for addition of a second antihypertensive agent at the time of Screening. Eligible patients must have a serum aldosterone ≥7 ng/dL (≥6 ng/dL if on an ACEi or ARB) and meet all other screening criteria.

Screening laboratory evaluations, if abnormal, may be repeated once for eligibility purposes before excluding the patient. Screen failures may be rescreened no less than 5 days after the last study visit, with Sponsor and/or Medical Monitor consultation and approval.

During the study, patients will complete between 8 to 10 scheduled visits, including 7 to 9 clinical site visits and 1 telephone visit. Unscheduled visits may be scheduled at any time during the study based on Investigator's discretion. The study will consist of the following periods/visits:
A Screening Period of at least 4 weeks consisting of:
   A Screening Visit (Visit 1);
   A Telephone Visit to convey patient eligibility for the study; and
   A Run-In Period up to 4 weeks before randomization (Visit 2), to confirm the patient's adherence to their background antihypertensive medication and placebo (see Inclusion Criterion 4).
A 2-part Treatment Period consisting of:
   Part 1: A double-blind Treatment Period of 8 weeks (Weeks 1 to 8; Visits 2 to 6); and
   Part 2: A Treatment Period of 4 weeks (Weeks 9 to 12; Starting the day after Visit 6 through Visit 9).
A Safety Follow-Up Period (Visit 10) of approximately 2 weeks after the last dose of study drug.

Upon return of the screening eligibility laboratory results including serum aldosterone, patients will be contacted via telephone (Telephone Visit) to inform them of their eligibility, and if eligible, to begin the Run-In Period and schedule their next visit: an Unscheduled Visit or Visit 2. For patients with serum sodium <130 mEq/L and/or serum potassium >5 mEq/L at Screening that the Investigator elects to correct or manage, 1 retest (at an Unscheduled Visit) is allowed at least 1 week prior to Visit 2 (see Exclusion Criterion 21). Eligible patients will also be instructed to begin the Run-In Period when they will take the placebo tablet once daily (QD) in addition to their background antihypertensive agent until Visit 2. A patient who demonstrates treatment adherence (see Inclusion Criterion 4) and continues to satisfy all inclusion criteria and none of the exclusion criteria at Visit 2 will be randomized and enter the Part 1 Treatment Period.

Clinical sites will provide patients with a 24-hour urine collection kit at Visits 1, 5, and 8. Patients will be instructed to start the collection up to 3 days prior to Visits 2 (after confirmation of their eligibility during the Telephone Visit), 6, and 9, refrigerate the collected sample, and bring the entire sample to the clinical site at that visit.

During the double-blind Part 1 Treatment Period (Weeks 1 to 8; Visits 2 to 6), patients will be randomized (1:1:1:1) to 1 of 4 treatment groups: 0.5 mg (R)-compound 1, 1 mg (R)-compound 1, 2 mg (R)-compound 1, or placebo, as add-on medications to their single background antihypertensive agent. On clinical site visit days, patients will self-administer the morning dose of background antihypertensive medications at home and withhold the study drug. At the clinical site, patients will self-administer 1 tablet of study drug to be witnessed by site staff, after completion of pre-dose evaluations and laboratory sampling. Between clinical site visits, patients will continue to self-administer 1 tablet of study drug QD by mouth at approximately the same time each morning. The endpoint will be evaluated at the end of Week 8.

During the Part 2 Treatment Period (Weeks 9 to 12; starting the day after Visit 6 and running through Visit 9), all responders (defined as achieving a mean seated SBP<130 mmHg) at the end of Part 1 will move into Part 2. They will receive the 2 mg dose of (R)-compound 1 (maximum in this study) and discontinue their single background antihypertensive agent. Non-responders who received any study drug except 2 mg (R)-compound 1 in Part 1 will move into Part 2, receive the 2 mg dose of (R)-compound 1, and discontinue their single background antihypertensive agent. A non-responder who decides not to participate in Part 2 or has already received the maximum dose strength (2 mg) of (R)-compound 1 in Part 1 will be considered withdrawn from the study drug, and should complete their EOT (Visit 9) procedures at the end of Part 1 (Visit 6) and the 2-week Safety Follow-Up.

Patients will be instructed to bring their study drug and background antihypertensive medication to all clinical site visits. Patients should not exercise, smoke, or consume caffeinated beverages or food for at least 2 hours prior to each clinical site visit. All clinical site visits should occur at approximately the same time (intra-patient) and efforts should be made to have the visits occur between 6:00 AM and 11:00 AM.

During Safety Follow-Up (Visit 10), patients will be evaluated for vital signs, clinical laboratory assessments, adverse events (AEs), and concomitant medication use including antihypertensive regimen since study completion.

The safety of (R)-compound 1 will be assessed from the time of informed consent until the end of the Safety Follow-Up Period. Patients will be followed for efficacy and adherence as prespecified during the Treatment Period. PD variables analyzed during the study may include, but are not limited to, measures of aldosterone and its precursors, cortisol and its precursor, plasma renin activity (PRA), and calculation of aldosterone/PRA ratio. PK variables analyzed during the study will include plasma concentrations of (R)-compound 1 and any measured metabolite(s). A Data Safety Monitoring Board (DSMB) is planned to periodically evaluate emerging safety data and assess reports on cumulative SAEs.

### Dosage Forms and Route of Administration

The dose strengths of (R)-compound 1 are 0.5 mg QD, 1 mg QD, and 2 mg QD. For the Run-In Period, all patients will self-administer 1 tablet of placebo QD by mouth at approximately the same time each morning.

During clinical site visits for the Treatment Period (Parts 1 and 2), patients will self-administer 1 tablet of study drug in the clinic to be witnessed by site staff, after completion of pre-dose evaluations and laboratory sampling. Between clinical site visits, patients will continue to self-administer 1 tablet of study drug QD by mouth at approximately the same time each morning.

### Endpoints

An efficacy endpoint is the change from baseline in mean seated SBP after 8 weeks of treatment in patients with uncontrolled HTN and a higher serum aldosterone level receiving 1 background antihypertensive agent (Part 1).

Other efficacy endpoints of this study in the same population as that described for the endpoint are as follows:
The change from baseline in mean seated DBP with (R)-compound 1 compared to placebo after 8 weeks of treatment (Part 1);
The change from baseline in 24-hour urine aldosterone and serum aldosterone levels with (R)-compound 1 compared to placebo after 8 weeks of treatment (Part 1); and
The percentage of patients achieving a mean seated SBP <130 mmHg (“responders”) with (R)-compound 1 compared to placebo after 8 weeks of treatment (Part 1; Weeks 1 to 8).

Other efficacy endpoints of this study are as follows:
The change in 24-hour urine aldosterone and serum aldosterone levels from values measured at the end of Part 1 to those measured following 4 weeks of treatment with (R)-compound 1 2 mg dose strength and no background antihypertensive agent at the end of Part 2; and
The percentage of patients maintaining a mean seated SBP <130 mmHg (“responders”) when treated with (R)-compound 1 2 mg alone and no background antihypertensive agent for 4 weeks during Part 2.

### Safety Endpoints

The safety endpoints of this study are as follows:
Vital signs, standing BP and heart rate, physical examinations, ECG, body weight, and clinical laboratory assessments, including standard safety chemistry panel, hematology, coagulation, and urinalysis;
TEAEs;
TESAEs;
TEAEs leading to premature discontinuation of study drug;
Treatment-emergent marked laboratory abnormalities; and
Change in standing SBP (measured pre-dose at the clinical site) from baseline to EOT (Visit 9).

### Safety Analysis

The Safety Population will be the population for the safety analysis. All safety endpoints will be summarized descriptively for records collected in Part 1. Additional safety endpoint analyses will be conducted including records collected in Part 2 and the post-dose follow up/end of study.

### Pharmacokinetic Analysis

Individual plasma concentration data for (R)-compound 1 and any measured metabolite(s) will be listed and summarized by visit, timepoint, and treatment group for the PK Population.

For patients participating in Part 2, relevant parameters for (R)-compound 1 and any measured metabolite(s) will be listed by individual patient and summarized in tabular format using descriptive statistics. Mean and individual plasma concentrations of (R)-compound 1 and any measured metabolite(s) will be plotted against time points for patients in Part 2.

### Pharmacodynamic Analysis

The PD Population will be the population for the PD analysis. All PD variables will be summarized descriptively.

### Pharmacokinetic-Pharmacodynamic Analysis

An attempt will be made to correlate plasma concentrations and parameters with measures of safety, PD, and/or efficacy, if the data permit.

### Formulation and Packaging

(R)-compound 1 tablets will be provided in the following dose strengths: 0.5 mg, 1 mg, and 2 mg. The tablets will be packaged in blister packs to achieve the doses required for the study. (R)-compound 1 tablets will contain (R)-compound 1 as the active ingredient and inactive ingredients.

### Example 4

A Randomized, Double-Blind, Placebo-Controlled, Multicenter, Parallel-Group, Dose-Ranging Study to Evaluate the Efficacy and Safety of (R)-compound 1 for the Treatment of Patients with Primary Aldosteronism (PA).

### Indication

Reduction of Blood Pressure (BP) in Patients with Primary Aldosteronism (PA)

### Objectives

One objective is to demonstrate that at least one dose strength of (R)-compound 1 is superior to placebo in mean change from baseline in seated systolic blood pressure (SBP) by automated office blood pressure monitoring (AOBPM) after 4 weeks of treatment in patients with PA.

Other objectives are the following:
To evaluate the change from baseline in mean seated diastolic blood pressure (DBP) by AOBPM, with each of the selected dose strengths of (R)-compound 1 compared to placebo, after 4 weeks of treatment in patients with PA;
To evaluate the percentage of patients achieving a seated BP response <140/90 mmHg, with each of the selected dose strengths dose of (R)-compound 1 compared to placebo, after 4 weeks of treatment for PA;
To evaluate the percentage of patients achieving a seated BP response <130/80 mmHg, with each of the selected dose strengths of (R)-compound 1 compared to placebo, after 4 weeks of treatment for PA;
To evaluate the percentage of patients with each of the selected dose strengths of (R)-compound 1 compared to placebo, after 4 weeks of treatment for PA who achieve either:
   a plasma aldosterone concentration (PAC)<15 ng/dL and a plasma renin activity (PRA) ≥0.5 ng/mL/h; or
   an aldosterone-to-renin ratio (ARR)<30; and
To evaluate the change from baseline in potassium levels and/or potassium supplementation requirements, with each of the selected dose strengths of (R)-compound 1 compared to placebo, after 4 weeks of treatment in patients with PA.

Other objectives are the following:
To evaluate the changes in the concentrations from baseline to End of Treatment of
pharmacodynamic (PD) variables, including but not limited to: PAC, 11-deoxycorticosterone,
PRA, direct renin concentration, calculated aldosterone-to-renin ratio (ARR), and 24-hour urinary aldosterone, sodium, and potassium, with each of the selected dose strengths of
(R)-compound 1 compared to placebo, after 4 weeks of treatment in patients with PA; and
To evaluate the relationship between BP reduction and changes in aldosterone and renin levels, with each of the selected dose strengths of (R)-compound 1 compared to placebo, from baseline to End of Treatment.

### Pharmacokinetic PK/PD Objectives

The pharmacokinetic (PK)-PD objective is to evaluate the exposure-response relationships of (R)-compound 1 in patients with PA using measures of safety, PD, and/or efficacy.

### Inclusion Criteria

Patients who meet all the following criteria are eligible to participate:
1. Is an adult male or female patient ≥18 years of age;
2. Has a prior diagnosis of PA or a suspected diagnosis of PA per the Investigator's judgment;
3. Is able to wash out of renin-angiotensin-aldosterone system (RAAS)-modifying drug(s) beginning at Visit 2 (if applicable), for at least the following durations prior to returning for Visit 3:
4 weeks: diuretics, mineralocorticoid receptor antagonists (MRAs); and
2 weeks: beta blockers, clonidine, methyldopa, minoxidil, nonsteroidal anti-inflammatory drugs (NSAIDs), angiotensin-converting enzyme inhibitors (ACEIs), angiotensin receptor blockers (ARBs), and/or dihydropyridine calcium channel blockers (CCBs);
Patients will remain off of all RAAS-modifying drug(s) beginning at Visit 2 and through the End of Treatment. Patients may be placed on non-RAAS-modifying antihypertensive drug(s), defined as mono- or combination therapy with a nondihydropyridine CCB (e.g., diltiazem, verapamil), hydralazine, or an alpha blocker, irrespective of their washout status, so as to maintain BP>130/80 mmHg and <160/100 mmHg for the remainder of the Screening Period and to minimize the effect on PAC and PRA. Patients taking beta-blockers should be managed appropriately by the Investigator during washout which should include gradual down-titration and observation of these patients for withdrawal symptoms (e.g., palpitations, chest pain, etc.)
4. Is willing to have BP maintained >130/80 mmHg and <160/100 mmHg during the Screening Period and <160/100 mmHg during the Double-Blind Treatment Period with the use of allowable non-RAAS-modifying antihypertensive drug(s);
5. Is willing to be compliant with the contraception and reproduction restrictions of the study as follows:
Male subjects must agree to abstain from sperm donation from Day 1 through 90 days after the final dose of study drug;
Postmenopausal women must have not had menstrual bleeding for at least 1 year before initial dosing and either be >60 years or have an elevated follicle-stimulating hormone (FSH) level >40 mIU/mL at the Screening Visit;
Female patients of childbearing potential (i.e., ovulating, pre-menopausal, and not surgically sterile) must have a documented negative pregnancy test at the Screening and Randomization Visits; and
All male patients (unless surgically sterile) must use a highly effective method of
contraception (i.e., <1% failure rate) from Day 1 through 90 days after the last administration of study drug;
Acceptable methods of contraception for male patients enrolled in the study include the following:
   Condoms with spermicide; or
   Surgical sterilization (vasectomy) at least 26 weeks before the Screening Visit
Female patients of childbearing potential must use a highly effective method of contraception
(i.e., <1% failure rate) from Day 1 through 30 days after the last administration of study drug;
Acceptable methods of contraception for FPCBP enrolled in the study include the following:
   Surgical sterilization (tubal ligation);
   Intrauterine device for at least 12 weeks before the Screening Visit;
   Hormonal contraception (oral, implant, injection, ring, or patch) for at least 12 weeks before the Screening Visit; or
Diaphragm used in combination with spermicide; and
6. Is able and willing to give informed consent for participation in the study.

### Exclusion Criteria

Patients who meet any of the following criteria are excluded from participation in the study:
1. Has a single occurrence of mean seated SBP>180 mmHg or DBP>110 mmHg or has 2 consecutive occurrences of mean seated SBP≥160 mmHg or DBP≥100 mmHg by AOBPM during the Screening Period (If such a BP is recorded at Visit 1, the patient will attend an Interim Visit for additional an BP measurement and reassessment of Inclusion/Exclusion Criteria);
Mean seated BP is defined as the average of 3 measurements obtained at any 1 clinical site visit. If the patient missed the regularly scheduled antihypertensive medication(s) prior to the visit (Visits 1, 2, 3, or 4), and in the opinion of the Investigator has been otherwise adherent to their antihypertensive regimen, 1 BP re-test is allowed ≥2 hours after taking medication(s), or on the following day, or later after reestablishing the regularly scheduled antihypertensive regimen.
2. Has a body mass index >40 kg/m² at the Screening Visit;
3. Has an upper arm circumference <7 or >17 inches at the Screening Visit;
4. Has had a previous surgical intervention or has a planned surgical intervention for an adrenal adenoma or adrenal carcinoma, adrenalectomy, renal nerve denervation, or adrenal ablative procedure during the course of the study; Patients who have had a procedure >6 months prior to randomization but still have elevated PAC (>15 ng/dl) and meet BP and other eligibly criteria may be considered.
5. Has a documented estimated glomerular filtration rate <45 mL/min/1.73 m² using the Chronic Kidney Disease Epidemiology equation at the Screening Visit or at Visit 4;
6. Has a planned dialysis or kidney transplantation during the course of the study;
7. Has known documented chronic heart failure;
8. Has had a stroke, transient ischemic attack, hypertensive encephalopathy, acute coronary syndrome, or hospitalization for heart failure within 6 months before the Screening Visit;
9. Has known current severe left ventricular outflow obstruction, such as obstructive hypertrophic cardiomyopathy and/or severe aortic valvular disease, diagnosed from a prior echocardiogram;
10. Has a planned coronary revascularization (percutaneous coronary intervention [PCI] or coronary artery bypass graft [CABG]) or any major surgical procedure during the study;
11. Has had PCI, CABG, other major cardiac surgery (e.g., valve replacement), or peripheral arterial bypass surgery within 6 months before the Screening Visit;
12. Has chronic permanent atrial fibrillation;
13. Has a history of, or currently experiencing, clinically significant arrhythmias as judged by the Investigator, including ventricular tachycardia, ventricular fibrillation, Torsades de points, and supraventricular arrhythmias. Patients with minor forms of ectopy (e.g., premature atrial contractions) are not necessarily excluded, per Investigator discretion;
14. Has had a prior solid organ transplant or cell transplant;
15. Is expected to receive or is receiving any of the exclusionary drugs such as strong inducers of cytochrome P450 3A, drugs known to prolong QT, and/or chronic use of NSAIDs or steroids within 28 days or 5 half-lives, whichever is longer prior to the first dose of study drug until the end of treatment; patients who are using medication(s) noted above at Screening who are willing to come off during the course of the study are allowed to participate.
16. Has a known hypersensitivity to any of the following:
Compound 1 or drugs of the same class;
Captopril or drugs of the same class; or
Excipients in (R)-compound 1, captopril, or drugs of the same classes;
17. Has any clinically relevant medical or surgical conditions (including unstable conditions and/or conditions requiring treatment with systemic immunosuppressants, including corticosteroids) that, in the opinion of the Investigator, would put the patient at risk by participating in the study;
18. Has evidence of any of the following at the Screening, Visit 4, or the Randomization Visit:
White blood cell count >15×10⁹/L or absolute neutrophil count <1×10⁹/L;
Serum potassium <2.5 mEq/L;
Patients with a serum potassium level below normal range may continue in the study if the Investigator elects to correct the serum potassium level with supplementation and offers to manage the condition.
Serum potassium >5.0 mEq/L;
Applies to Visit 4 and Randomization only. Patients with a serum potassium level >5.0 mEq/L at Visit 1 may continue in the study if the Investigator elects to correct the serum potassium level and/or offers to manage the condition while the patient washes out of RAAS-modifying drug(s).
Hemoglobin <10.0 g/dL or anticipated initiation of erythropoietin-stimulating agents or planned transfusion within 2 months after the Screening Visit;
Serum aspartate aminotransferase or alanine aminotransferase >3×upper limit of normal (ULN); or
Total bilirubin >2×ULN, unless due to Gilbert's syndrome;
19. Has uncontrolled diabetes with glycosylated hemoglobin >9.5% at the Screening Visit;
20. Is positive for HIV antibody, hepatitis C virus RNA, or hepatitis B surface antigen;
21. Has typical consumption of >14 alcoholic drinks weekly; 1 drink of alcohol is equivalent to 12 pint of beer (285 mL), 1 glass of spirits (25 mL), or 1 glass of wine (125 mL).
22. Has participated in another clinical study involving any investigational drug within 30 days prior to the Screening Visit, or plans to participate in another clinical study within 30 days of discontinuation of study drug;
23. Has received experimental therapy with a small molecule within 30 days of the Screening Visit or 5 half-lives, whichever is longer, or received experimental therapy with a large molecule within 90 days of the Screening Visit or 5 half-lives, whichever is longer;
24. Has been on night shifts at any time during the 4 weeks before the Screening Visit and/or plans to begin night shift at any time during the Screening and/or Double-Blind Treatment period;
25. Is pregnant, breastfeeding, or planning to become pregnant during the study; or
26. Is considered by the Investigator, after reviewing medical and psychiatric history, physical examination, and laboratory evaluations, to be unsuitable for any other reason that may either place the patient at increased risk during participation or interfere with the interpretation of the study outcomes.

### Study Design and Duration

This is a Phase 2, randomized, placebo-controlled, multicenter, parallel-group, dose-ranging study in patients with PA to evaluate the efficacy and safety of the selected dose strengths of (R)-compound 1 as compared to placebo after 4 weeks of treatment. Adult patients with either a prior diagnosis of PA or a suspected diagnosis of PA are eligible to screen for study participation.

The safety of (R)-compound 1 will be assessed from the time of informed consent until the end of the Follow-Up Period. Patients will be followed for efficacy and adherence throughout the Double-Blind Treatment Period. Plasma PD variables analyzed during the study will include measures of aldosterone and its relevant precursors, cortisol and its relevant precursor, PRA, direct renin concentration, and calculated ARR. PK variables analyzed during the study will include plasma concentrations of (R)-compound 1 and any additional metabolite(s).

Patients will be instructed to bring their medications (antihypertensive drugs [if applicable] and study drug) and daily paper diary to all clinical site visits for assessing treatment adherence and for reviewing home BP monitoring, respectively. Patients should not exercise, smoke, or consume caffeinated beverages or food for at least 2 hours prior to each clinical site visit. All clinical site visits should occur between 6:00 a.m. and 11:00 a.m. and, when possible, at the same time for each visit.

### Study Visits

Patients will complete at least 10 visits over a period of approximately 7 to 15 weeks, including 9 clinic visits and 1 telephone visit. Additional interim visits may occur as per the Investigator's discretion to manage BP during the Screening period.

The study will consist of the following 3 periods and corresponding visits:
A Screening Period of approximately 2 to 9 weeks will comprise the following:
   Screening Visit (Visit 1);
   Washout Visit (Visit 2);
      beginning after Visit 2, patients will begin washout of applicable RAAS modifying drug(s) for at least the following durations prior to returning for Visit 3:
         4 weeks: diuretics, mineralocorticoid receptor antagonists (MRAs); and
         2 weeks: beta blockers, clonidine, methyldopa, minoxidil, nonsteroidal anti-inflammatory drugs (NSAIDs), angiotensin-converting enzyme inhibitors (ACEIs), angiotensin receptor blockers (ARBs), and/or dihydropyridine calcium channel blockers (CCBs);
         Patients will remain off of all RAAS-modifying drug(s) beginning as Visit 2 and through End of Treatment. Patients may be placed on non-RAAS-modifying antihypertensive drug(s), defined as mono- or combination therapy with a nondihydropyridine CCB (e.g., diltiazem, verapamil), hydralazine, or an alpha blocker, irrespective of their washout status, so as to maintain BP>130/80 mmHg and <160/100 mmHg during the remainder of the Screening Period and to minimize the effect on PAC and PRA.
         Patients taking beta-blockers should be managed appropriately by the Investigator during washout which should include gradual down-titration and observation of these patients for withdrawal symptoms (e.g., palpitations, chest pain, etc.).
         Patients who are not taking RAAS-modifying drug(s) at Visit 1 will not be required to complete Visit 2 and may proceed directly to Visit 3.
   Bloodwork Visit (Visit 3); and
   Confirmatory Test Visit (Visit 4);
      Additional Interim Visits may occur as needed in order to manage BP during the wash out of RAAS-modifying drug(s).
A Double-Blind Treatment Period (Visits 5 to 9) of 4 weeks;
   Patients entering the Double-Blind Treatment Period must remain on their stable non-
   RAAS antihypertensive regimen (as applicable) and maintain blood pressure <160/100 mmHg throughout treatment; and
   A Follow-Up Period (Telephone Call [Visit 10]) of up to 1 week.

### Screening Period

Patients who provide written informed consent at Visit 1 will be assessed for the

### Inclusion/Exclusion Criteria.

At Visit 1, site staff will measure BP; obtain blood samples for PAC, PRA, and direct renin concentration measurement; and perform routine safety evaluations. Patients will use a daily electronic diary to monitor adherence to their antihypertensive regimen (if applicable) throughout the screening period.

Starting at Visit 2, patients will be required to wash out of their current RAAS-modifying drug(s), if applicable, and will remain off these for the entire duration of study participation. A ≥4-week washout prior to Visit 3 is required if the patient is taking diuretics, including MRAs. A ≥2-week washout prior to Visit 3 is required if the patient is taking beta blockers, clonidine, methyldopa, minoxidil, NSAIDs, ACEIs, ARBs, and/or dihydropyridine CCBs. Patients taking beta-blockers should be managed appropriately by the Investigator during washout which should include gradual down-titration and observation of these patients for withdrawal symptoms (e.g., palpitations, chest pain, etc.)

Patients may be placed on non-RAAS-modifying antihypertensives defined as mono- or combination therapy with a non-dihydropyridine CCB (e.g., diltiazem, verapamil), hydralazine, or an alpha blocker, irrespective of their washout status. Patients will have the option to receive generic non-RAAS-modifying antihypertensive drug(s), through a Central Pharmacy during the study period. During the Screening Period, Interim Visits may be scheduled to check BP status as new antihypertensive agents are introduced. Patients will be provided a home BP monitoring device at Visit 2 for monitoring their BP at home each morning and evening throughout the study.

Patients who are not taking RAAS-modifying drug(s) at Visit 1 will not be required to complete Visit 2 (Washout Visit) and may proceed directly to Visit 3.

Patients will present for Visit 3 after completion of the applicable washout period. At Visit 3, site staff will measure BP, perform routine safety evaluations, and obtain blood samples for PAC, PRA, and direct renin concentration measurement. Patients with either (1) a PAC ≥15 ng/dL and a PRA <0.5 ng/mL/h or (2) an ARR ≥30 are eligible to proceed to Visit 4.

At Visit 4, a seated captopril challenge will be administered as the confirmatory test for PA.

Patients will receive a single oral dose of captopril 50 mg after being seated for at least 1 hour (Time 0). Blood samples for PAC, PRA, and cortisol will be collected at Time 0 and hours 1 and 2 after captopril administration while the patient continues to remain seated during this sampling period. Patients with PAC suppression <30% at hours 1 or 2 compared to Time 0, and/or a PAC >11 ng/dL at hours 1 or 2 after Time 0 of the seated captopril challenge are eligible to proceed to Randomization (Visit 5).

The length of the Screening Period may need to be extended to ensure that the patient is on a stable (≥2 week) non-RAAS antihypertensive regimen with BP>130/80 mmHg and <160/100 mmHg prior to the Randomization Visit (Visit 5). The time from Visit 3 to Visit 5 can be included in the 2-week stable BP period.

A patient who is screened and does not meet the study Inclusion/Exclusion Criteria or Randomization Criteria (i.e., Screen Failure) may be rescreened no less than 5 days after the last study visit, with Sponsor and/or Medical Monitor consultation and approval. Patients who screen fail based on the results of the captopril challenge at Visit 4 may not be rescreened.

### Double-Blind Treatment Period

Measurements obtained at Visit 5 prior to the patient taking the study drug will constitute “baseline” measurements. Measurements of efficacy and safety variables recorded prior to study drug administration at the clinical site will constitute “pre-dose” measurements.

### Adaptive Design

At Visit 5, eligible patients will be randomized 1:1:1 into 1 of the 3 treatment groups (2 active [2 mg and 4 mg (R)-compound 1] and 1 placebo). Randomized patients will be stratified by baseline PAC (<35 ng/dL and >35 ng/dL). Patients will remain on the applicable non RAAS antihypertensive regimen and must maintain blood pressure <160/100 mmHg during the Double-Blind Treatment Period. After approximately 10 randomized patients per group complete the 4-week Double-Blind Treatment Period, an interim analysis will be performed, and a Data Review Committee (DRC) will evaluate emerging safety and efficacy data. Study enrollment is planned to continue during the interim analysis. Based on the unblinded safety and efficacy assessments, the DRC may decide to expand either or both of the current treatment groups, continue with 1 of the treatment groups (e.g., 2 mg QD or 4 mg QD (R)-Compound 1), and potentially one additional dose level of up to 8 mg QD (R)-compound 1. Following the interim analysis, patients will be enrolled using a randomization schedule that will allow for approximately equal distribution between the various treatment groups (2, 3, or 4 treatment groups) in accordance with the power and sample size calculations determined by the interim analysis.

Based on ongoing safety monitoring of the study, additional DRC reviews may be conducted, which may or may not lead to a formal unblinded interim analysis. Details of DRC responsibilities, authorities, and procedures will be documented in the DRC Charter. Between clinical site visits, adherence to both the antihypertensive regimen (if applicable) and study drug will be monitored with the daily electronic diary. During clinical site visits, adherence to study drug and antihypertensive regimen (if applicable) will be calculated using pill counts.

Pre-dose blood samples for PD analysis will be collected at Visits 5 through 9. Pre-dose blood samples for PK analysis will be collected at Visit 6 and Visit 9. Post-dose blood sampling for PD and PK analysis will be performed at Visit 9 at 2 hours (+5 minutes) after study drug administration. Urine samples for PD and electrolyte measurements will be obtained over the 24 hours (24-hour urine collection) leading up to Visits 2 (Patients who are not taking RAAS modifying drug(s) at Visit 1 will not be required to complete collection of a 24-hour urine sample as these patients will not be required to complete Visit 2), 5, and 9/End of Treatment. The efficacy endpoint evaluation will take place at the End of Treatment (Visit 9).

### Follow-Up Period (Telephone Call)

Patients will receive a Telephone Call from the clinical site at 1 week±3 days following the last dose of the study drug to assess adverse events (AEs) and concomitant medications since study completion.

### Dosage Forms and Route of Administration

The (R)-compound 1 doses to be tested in this study are 2 mg QD, 4 mg QD, and optionally one additional dose level of up to 8 mg QD. (R)-Compound 1 will be provided as 2 mg tablets. Placebo tablets will be indistinguishable from the (R)-compound 1 tablets. The study drug will be stored at controlled room temperature of 20° C. to 25° C. (68° F. to 77° F.). Consistent with the United States Pharmacopeia (USP) references, excursions between 15° C. to 30° C. are allowed during storage. During transport, excursions up to 40° C. permissible up to 1 week. The intended route of administration to patients is by oral delivery. In order to maintain the study blind, randomized patients will be instructed to take a total of 4 tablets, comprised of (R)-compound 1 and/or placebo tablets, by mouth once daily (QD).

Study drug ((R)-compound 1 or placebo) will be dispensed at Visit 5 and Visit 7. At Visit 5, patients will self-administer the first single dose of study drug at the clinical site. Subsequent doses of the study drug are to be taken by the patient once daily by mouth at approximately the same time each morning at home. On days of clinical site visits, patients will take their morning dose of applicable antihypertensive drugs (including medications for other comorbidities, if any) at home prior to their scheduled visit and withhold the study drug. At the clinical site, patients will self-administer the study drug to be witnessed by site staff after completion of pre-dose evaluations and laboratory sampling.

### Efficacy Endpoints

An efficacy endpoint is the change from baseline in mean seated SBP by AOBPM after 4 weeks of treatment in patients with PA.

Other efficacy endpoints include the following:
The change from baseline in mean seated DBP by AOBPM, with (R)-compound 1 compared to placebo, after 4 weeks of treatment in patients with PA;
The percentage of patients who achieve a seated BP response <140/90 mmHg, with (R)-compound 1 compared to placebo, after 4 weeks of treatment for PA;
The percentage of patients who achieve a seated BP response <130/80 mmHg, with (R)-compound 1 compared to placebo, after 4 weeks of treatment for PA; and
The percentage of patients with (R)-compound 1 compared to placebo, after 4 weeks of treatment for PA who achieve either:
   a PAC <15 ng/dL and a PRA≥0.5 ng/mL/h; or
   an ARR<30.

Other endpoints include the following:
Changes in the concentrations from baseline to End of Treatment in PD markers, including but not limited to, PAC, 11-deoxycorticosterone, PRA, direct renin concentration, calculated ARR, and 24-hour urinary aldosterone, B-type Natriuretic Peptide, sodium, and potassium, with (R)-compound 1 compared to placebo, after 4 weeks of treatment; and
Relationship between BP reduction and changes in aldosterone and renin levels, with (R)-compound 1 compared to placebo, from baseline to End of Treatment.

### Safety Endpoints

The safety of (R)-compound 1 will be assessed from the time of informed consent until the end of the Follow-Up Period. All safety endpoints will be summarized descriptively. The safety endpoints will include the following:
Change from baseline in potassium levels and/or potassium supplementation requirements, with (R)-compound 1 compared to placebo, after 4 weeks of treatment in patients with PA;
Vital signs (heart rate, respiratory rate, body temperature), mean SBP, mean DBP, orthostatic vitals (standing BP and heart rate), physical examinations, electrocardiography, weight measurement, and clinical laboratory evaluations including standard safety chemistry panel, hematology, coagulation, and urinalysis;
Treatment-emergent adverse events (TEAEs);
Treatment-emergent serious adverse events (SAEs);
TEAEs leading to premature discontinuation of the study drug;
Treatment-emergent marked laboratory abnormalities; and
Change in standing SBP and DBP (measured at the clinical site prior to administration of study drug) from baseline to End of Treatment.

Adverse events of special interest will include the following which require clinical intervention: hypotension events, abnormal potassium laboratory values, and/or abnormal sodium laboratory values.

### Efficacy Analysis

An efficacy analysis will compare the change in mean seated SBP from baseline (Visit 5) to the End of Treatment (Visit 9) between each dose strength of (R)-compound 1 and placebo.

An efficacy endpoint will be analyzed with an analysis of covariance model with treatment group as a factor and with baseline mean seated SBP and baseline plasma aldosterone concentration as covariates. Pairwise comparisons between each dose strength of (R)-compound 1 and placebo, together with its 95% confidence interval, will be estimated. The efficacy analysis will be conducted based on the ITT Population, with the last observation carried forward method used to impute any missing endpoint values. Other imputation methods will be explored with missing at random or missing not at random assumptions. The endpoint analysis will be repeated on the PP Population to test the robustness of the results. A Mixed Model Repeated Measures (MMRM) method will be used in sensitivity analysis for the efficacy endpoint. Multiplicity will not be adjusted due to the exploratory nature of this study.

Other continuous efficacy endpoints will be summarized and analyzed with a similar model. Categorical efficacy endpoints will be summarized by frequency and percentage of defined responders and analyzed with logistic regression models.

The change in mean SBP, mean DBP, and potassium from baseline will be analyzed via a MMRM method for efficacy analysis. The analysis will include fixed effects for treatment, visit, and treatment-by-visit interaction, along with a covariate of the baseline value. The analysis will consist of only observed data (i.e., no imputation of missing data will be performed). No adjustment will be made for multiplicity in testing the efficacy endpoints.

### Safety Analysis

The Safety Population will be the population for the safety analysis. All safety endpoints will be summarized descriptively.

The assessment of safety will be based primarily on the frequency of AEs, clinical laboratory assessments, vital signs, and 12-lead electrocardiograms. Other safety data will be summarized as appropriate.

AEs will be coded using the Medical Dictionary for Regulatory Activities. TEAEs, defined as those AEs that newly occur or worsen in severity during the Double-Blind Treatment Period, will be summarized by system organ class and preferred term. A list of patients with SAEs and those who discontinue from the study due to an AE will be provided.

Summary statistics by treatment group at baseline, at each visit, and of changes from baseline to each visit for laboratory parameters, vital signs, and other safety measurements will be provided. The occurrence of significant abnormalities in change from baseline of laboratory values will be summarized by treatment group. Physical examination data will be listed.

### Pharmacokinetic Analysis

Individual plasma concentration data for (R)-compound 1 and any measured metabolite(s) will be listed and summarized by visit, timepoint, and treatment group.

### Pharmacodynamic Analysis

All PD variables will be summarized descriptively.

### Pharmacokinetic/Pharmacodynamic Analysis

An attempt will be made to correlate plasma concentration data with measures of safety, PD, and/or efficacy.

### Treatment Groups

At study initiation, eligible patients will be randomized 1:1:1 into 1 of the 3 treatment groups:
2 mg (R)-compound 1;
4 mg (R)-compound 1; or
Matching placebo.

After approximately 10 randomized patients per group complete the 4-week Double-Blind Treatment Period, a DRC will evaluate emerging safety and efficacy data and may decide to expand either or both of the current treatment groups, continue with 1 of the treatment groups (e.g., 2 mg or 4 mg (R)-compound 1), or add a treatment group of no more than 8 mg (R)-compound 1 with or without 1 of the prior treatment groups.

### Drug Supplies

(R)-Compound 1 will be provided as 2 mg tablets. (R)-compound 1 tablets will contain the active ingredient and inactive ingredients.

### Pharmacokinetic Assessments

Pre-dose PK samples will be collected within 15 minutes before study drug dosing. Post-dose samples will be collected approximately 2 hours ±5 minutes after study drug dosing. The actual date and time of collection of each PK sample will be recorded.

Samples for measurement of pre-dose concentration will be collected before study drug dosing at Visit 6 and Visit 9 of the Double-Blind Treatment Period. Samples for post-dose plasma concentrations at or near peak (R)-compound 1 levels will be collected after study drug dosing at Visit 9. Additional PK samples may also be collected in the event of an SAE, AE leading to withdrawal, or any other safety event at the discretion of the Investigator, DRC, and/or Sponsor, if needed for comparison with safety and tolerability data.

Samples will be analyzed to measure plasma concentrations of (R)-compound 1 and any measured metabolites using validated liquid chromatography mass spectrometry methods. Analysis will be performed by Medpace Bioanalytical Laboratories, LLC.

### Pharmacodynamic Assessments

### Screening Period

PD sampling will be performed during Visits 1, 2, 3, and 4. Patients with either (1) a PAC≥15 ng/dL and a PRA<0.5 ng/mL/h or (2) and ARR≥30 at Visit 3 are eligible to proceed to Visit 4. At Visit 4, as part of the seated captopril challenge, blood samples for PAC, PRA, and cortisol will be drawn at time 0 and at hours 1 and 2 after captopril administration, while the patient continues to remain seated during this sampling period. Patients with PAC suppression <30% at hours 1 or 2 compared to time 0, and/or a PAC >11 ng/dL at hours 1 or 2 are eligible to proceed to Randomization (Visit 5).

### Example 5

A Randomized, Double-Blind, Placebo-Controlled, Multicenter, Parallel-Group, Dose-Ranging Study to Evaluate (R)-compound 1 for the Treatment of Patients with Uncontrolled Hypertension and Chronic Kidney Disease (CKD).

### Indication

Treatment of hypertension in patients with uncontrolled hypertension and chronic kidney disease (CKD).

### Objectives

One objective of the study is to evaluate the treatment effect of (R)-compound 1 in systolic blood pressure (SBP) compared to placebo at week 26 in patients with uncontrolled hypertension and CKD.

Other objectives of the study are:
To evaluate the treatment effect of (R)-compound 1 at various dose strengths in SBP compared to placebo at week 26
To determine the percentage of patients achieving SBP<130 mmHg after 26 weeks of treatment by dosing strategy;
To evaluate the change from baseline in urinary albumin-to-creatinine ratio (UACR) with each dosing strategy of (R)-compound 1 compared to placebo after 26 weeks of treatment;
To evaluate the change from baseline in diastolic blood pressure (DBP) measured by AOBPM with each dosing strategy of (R)-compound 1 compared to placebo at Week 26;
To evaluate the change from baseline in estimated glomerular filtration rate (eGFR) after 26 weeks of treatment;
To evaluate the change from baseline with each dose strength of (R)-compound 1 compared to placebo after 26 weeks of treatment in patients with hypertension and CKD in pharmacodynamic (PD) variables
To evaluate the relationship between change in UACR and changes in aldosterone and renin levels with each dose strength of (R)-compound 1 compared to placebo after 26 weeks of treatment;
To evaluate the relationship between change in UACR and changes in blood pressure (BP) with each dose strength of (R)-compound 1 compared to placebo after 26 weeks of treatment;
To evaluate the relationship between the change in eGFR and changes in BP with each dose strength of (R)-compound 1 compared to placebo after 26 weeks of treatment

### Safety Objectives

The safety objectives of the study include:
To evaluate vital signs, standing BP and heart rate, physical examinations, electrocardiography, weight measurement, and clinical laboratory evaluations, including standard safety chemistry panel, hematology, coagulation, and urinalysis.
To evaluate treatment-emergent adverse events (TEAEs);
To evaluate treatment-emergent adverse events of interest (AEOI)
To evaluate TEAEs leading to premature discontinuation of study drug;
To evaluate treatment-emergent marked laboratory abnormalities; and
To evaluate the change in standing SBP and DBP (measured pre-dose at the clinical site) from baseline to End of Treatment (Visit 11).

### PK-PD Objective

The pharmacokinetic (PK)-PD objective of the study is to evaluate the exposure-response relationships of (R)-compound 1 in patients with uncontrolled hypertension and CKD using measures of safety, PD, and/or efficacy.

### Inclusion Criteria

Patients who meet all of the following criteria are eligible to participate in the study:
Is an adult male or female patient ≥18 years of age;
Has a mean seated SBP≥140 mmHg at Screening (Visit 1) and Visit 2;
Patients with mean seated SBP≥130 mmHg may be eligible if diabetic.

Mean seated SBP is defined as the average of 3 seated SBP measurements at any single clinical site visit.
Has a prior diagnosis of mild-to-severe CKD, defined as eGFR (based on the CKD-EPI equation) of 25 to 75 mL/min/1.73 m², inclusive, due to diabetic nephropathy and/or hypertensive nephrosclerosis at Visit 1;
All other types of CKD and known overlapping kidney diseases are exclusionary. To ensure patients with moderate and severe renal impairment will be represented, the number of patients with an eGFR ≥60 will be capped at 45 (approximately 15% of the total population.)
Has a UACR ≥200 mg/g (≥22.6 mg/mmol) in at least 2 out of 3 measurements based on first urine collected in the morning on consecutive days during the Screening Period;
Is currently taking an angiotensin-converting enzyme inhibitor (ACEi) or angiotensin receptor blocker (ARB) at the maximum tolerated daily dose, based on Investigator judgment, for >4 weeks prior to Visit 1;
It is expected that patients not currently taking an ACEi or ARB at Screening (Visit 1) will not initiate this class of medication during the entire Study Period.
If taking a sodium-glucose cotransporter-2 (SGLT2) inhibitor at Screening (Visit 1), the regimen must be stable for a period of at least 8 weeks before Visit 1 and is expected to remain at a stable dose over the study period;
It is expected that patients not currently taking an SGLT2 inhibitor at Screening (Visit 1) will not initiate this class of medication during the entire Study Period.
Is willing to be compliant with the contraception and reproduction restrictions of the study as follows:
Female patients of childbearing potential (i.e., ovulating, pre-menopausal, and not surgically sterile) must have a documented negative pregnancy test at Visit 1 and the Randomization Visit (Visit 3); and
Female patients of childbearing potential must use a highly effective method of contraception (i.e., <1% failure rate) from Day 1 through 30 days after the last administration of study drug.
   Acceptable methods of contraception for female patients of childbearing potential enrolled in the study include the following:
   Surgical sterilization (tubal ligation);
   Intrauterine device for at least 12 weeks before Visit 1;
   Hormonal contraception (oral, implant, injection, ring, or patch) for at least 12 weeks before Visit 1; or
   Diaphragm used in combination with spermicide.
Post-menopausal women must have not had menstrual bleeding for at least 1 year before initial dosing and either be >60 years or have an elevated follicle-stimulating hormone level >40 mIU/mL at Visit 1;
Is able and willing to give informed consent for participation in the study.
After the run-in period of 2 to 4 weeks, all patients must confirm the BP and UACR measurements still meet the eligibility criteria.

### Exclusion Criteria

Patients who meet any of the following criteria are excluded from participation in the study:
Have a documented diagnosis of type 1 diabetes;
Are not willing or not able to discontinue a mineralocorticoid receptor antagonist (MRA) or a potassium sparing diuretic as part of an existing antihypertensive regimen;

Patients taking an MRA or a potassium sparing diuretic (e.g., triamterene, amiloride, etc.) as an antihypertensive agent must be willing to discontinue this agent for study eligibility. The potassium sparing diuretic may be discontinued and replaced with a non-potassium sparing diuretic. All patients who remain on a stable regimen of antihypertensive agents, including a non-potassium sparing diuretic, for at least six weeks, are eligible to enter the single blind-Run In.
Have a single occurrence of mean seated SBP>180 mmHg or DBP>110 mmHg during the Screening Period (if such a BP is recorded during the Screening Period, the patient may attend an Interim Visit for an additional BP measurement and reassessment of Inclusion/Exclusion Criteria);

Mean seated BP is defined as the average of 3 measurements obtained at any 1 clinical site visit. If the patient missed the regularly scheduled antihypertensive medication(s) prior to the visit (Visits 1 or 2), 1 BP re-test is allowed ≥2 hours after taking the medication(s), on the following day, or later after reestablishing the regularly scheduled antihypertensive regimen.
Has a body mass index (BMI) >45 kg/m² at Visit 1;
Has documented bilateral clinically relevant renal artery stenosis of ≥70%;
Has had dialysis for acute kidney injury/acute renal failure within 12 weeks prior to the Screening Period, or has a planned dialysis or kidney transplantation during the course of the study;
Has known documented chronic heart failure New York Heart Association class III or IV and/or hospitalization for heart failure within 6 months of Visit 1;
Has had a stroke, transient ischemic attack, hypertensive encephalopathy, acute coronary syndrome, or hospitalization for heart failure within 6 months of Visit 1;
Has known current severe left ventricular outflow obstruction, such as obstructive hypertrophic cardiomyopathy and/or severe aortic valvular disease, diagnosed from a prior echocardiogram or another imaging study;
Has a planned coronary revascularization (percutaneous coronary intervention [PCI] or coronary artery bypass graft [CABG]) or any major surgical procedure during the study;
Has had PCI, CABG, other major cardiac surgery (e.g., valve replacement), or peripheral arterial bypass surgery within 6 months of Visit 1;
Has had a prior solid organ transplant or cell transplant;
Is expected to receive or is receiving any of the exclusionary drugs such as strong inducers of cytochrome P450 3A, chronic use of non-steroidal anti-inflammatory drugs, spironolactone/eplerenone, and/or chronic use of steroids;
Has a known hypersensitivity to any of the following:
(R)-compound 1 or drugs of the same class; or
Excipients in (R)-compound 1 or drugs of the same classes.
Has received immunotherapy for treatment of CKD within 6 months of Visit 1 or expects to receive immunotherapy for treatment of CKD during participation in the study;
Has any clinically relevant medical or surgical conditions including unstable conditions and/or conditions requiring regular transfusion or treatment with systemic immunosuppressants, including corticosteroids that, in the opinion of the Investigator, would put the patient at risk by participating in the study;
Has evidence of any of the following at Visit 1 or the Randomization Visit (1 retest is allowed):
White blood cell count >15×10⁹/L or absolute neutrophil count <1×10⁹/L;
Serum potassium <3.5 mEq/L;
Patients with a serum potassium level below normal range may continue in the study if the Investigator elects to correct the serum potassium level with supplementation and offers to manage the condition.
Serum potassium >5.0 mEq/L;
Serum sodium <130 mEq/L;
Hemoglobin <8.5 g/dL;
Serum aspartate aminotransferase or alanine aminotransferase >3×upper limit of normal (ULN); or
Total bilirubin >2×ULN, unless due to Gilbert's syndrome.
Has uncontrolled diabetes with glycosylated hemoglobin >10.5% at Visit 1.
Is positive for HIV antibody, hepatitis B surface antigen, or hepatitis C virus RNA;
Has typical consumption of >14 alcoholic drinks weekly;
1 drink of alcohol is equivalent to 12 pint of beer (285 mL), 1 glass of spirits (25 mL), or 1 glass of wine (125 mL).
Has participated in another clinical study involving any investigational drug within 30 days prior to Visit 1, or plans to participate in another clinical study within 30 days of discontinuation of study drug;
Has received experimental therapy for disease intervention with a small molecule within 30 days of Visit 1 or 5 half-lives, whichever is longer, or received experimental therapy with a large molecule within 90 days of the Visit 1 or 5 half-lives, whichever is longer;
Vaccinations, including those for Coronavirus Disease 2019 (COVID-19), will not be exclusionary if administered during the Screening Period.
Is pregnant, breastfeeding, or planning to become pregnant during the study; or
Is considered by the Investigator, after reviewing medical and psychiatric history, physical examination, and laboratory evaluations, to be unsuitable for any other reason that may either place the patient at increased risk during participation or interfere with the interpretation of the study outcomes.

### Study Design

This is a phase 2, randomized, double-blind, placebo-controlled, multicenter, parallel-group, dose-ranging study to evaluate the efficacy and safety of (R)-compound 1 for the treatment of hypertension in patients with uncontrolled hypertension and CKD.

The safety of (R)-compound 1 will be assessed from the time of randomization until the end of the Follow-Up Period. Patients will be followed for efficacy and adherence throughout the Double-Blind Treatment Period. Plasma, serum, and urine PD variables analyzed during the study will include measures of kidney function and aldosterone and its relevant precursors. PK variables analyzed during the study will include plasma concentrations of (R)-compound 1 and any additional metabolite(s).

Patients will be instructed to bring their study drug to all clinical site visits after randomization for assessing treatment adherence.

### Study Visits

Patients will complete at least 10 visits over a period of approximately 8 months.

The study will consist of the following 3 periods and corresponding visits:
A Screening Period of up to 4 weeks (Visits 1 and 2) including a 2-week Run-In Period (beginning at Visit 2 until Visit 3);
A Double-Blind Treatment Period of 26 weeks (Visits 3 through 9); and
A Follow-Up Period of 2 weeks (Visit 10).

### Screening Period Including the 2-Week Run-In Period

Patients who provide written informed consent at Visit 1 will be assessed for the Inclusion/Exclusion Criteria.

At Visit 1, site staff will measure BP and vitals, obtain blood samples for eGFR and PD marker assessment, and perform routine safety evaluations and a dipstick urinalysis pre-screen to exclude patients who are negative for albuminuria. Patients will be provided with materials to obtain urine via first morning void at their home on 2 consecutive days prior to and on the morning of Visit 2 (Days −21 to −7). At least 4 days prior to Visit 2, site staff will place a reminder call instructing the patient to obtain the samples on the following 3 consecutive mornings. The third sample will be collected such that the date of collection and Visit 2 are the same.

At Visit 2, patients will bring the collected urine samples to the clinical site and site staff will send the samples to a central laboratory for UACR determination. Site staff will assess Inclusion/Exclusion Criteria, measure BP, obtain vitals, and assess concomitant medications. Patients will be provided with materials and instructions to collect 24-hour urine beginning the morning of Day −1. On Day −3 (+1 day), site staff will place a reminder call to the patient to obtain the 24-hour urine such that the date of completion of the 24-hour period and Visit 3 (Randomization Visit) are the same.

A patient who has consented to participate in the study but does not meet the study Inclusion/Exclusion Criteria (i.e., Screen Failure) may be rescreened no less than 5 days after the last study visit, with Sponsor and/or Medical Monitor consultation and approval.

At Visit 2, the patients will be provided with 2-week supplies of single-blind (R)-compound 1 placebo run-in drug and instructions on lifestyle management, reminders concerning hydration and the expectation that they will continue their background anti-hypertensive medications and, if relevant, SGLT2 inhibitor.

Upon return of the screening eligibility laboratory results of UACR, patients will be contacted via telephone to inform them of their eligibility, and if eligible, to begin taking the study drug once per day and schedule their next visit (Visit 3).

### Double-Blind Treatment Period

At Visit 3 (Randomization Visit), inclusion criteria for SBP and UACR will be confirmed. Patients who remain eligible will be randomized 1:1:1 into 1 of the 3 treatment groups (compound 1 0.5 mg tablets, (R)-compound 1 1 mg tablets and (R)-compound 1 placebo tablets). Randomization will be stratified by SGLT2 inhibitor use, baseline SBP (≤155 mmHg or >155 mmHg) and CKD category (eGFR≤45 mL/min/1.73 m² or >45 mL/min/1.73 m²).

The (R)-compound 1 dose levels may be up-titrated within the first 8 weeks after the day of randomization. At week 2 (visit 4), blood pressure will be measured, and a blood sample will be drawn for serum electrolyte measurements. The dose may be up titrated at week 4 (Visit 5) if a patient does not achieve SBP<130 mmHg target and does not experience hyperkalemia or hyponatremia based on samples drawn at week 2.

At week 8 (visit 6), blood pressure will be measured, and a blood sample will be drawn for serum electrolyte measurements. The dose may be up-titrated at week 8 if a patient does not achieve SBP<130 mmHg target and does not experience hyperkalemia or hyponatremia based on samples drawn at week 4. No further dose titration is permitted after week 8. Down titration is not permitted in this study.

For UACR measurements, baseline is defined as geometric mean of the 3 samples returned at Visit 2. For BP measurements, baseline is defined as the average of the 3 measurements taken prior to randomization at Visit 3. Measurements of efficacy and safety variables recorded prior to the first dose of double-blind study drug administration will constitute pre-dose measurements.

During clinical site visits, adherence to study drug dosing will be calculated using pill counts. Pre-dose blood samples for PD will be collected at Visits 3 through 9. Pre-dose blood samples for PK analysis will be collected at Visits 7 and 9. PD and PK blood samples will be collected within approximately 15 minutes prior to dosing.

Urine samples for PD and electrolyte measurements will be obtained over 24 hours (24-hour urine collection) leading up to Visit 3 (baseline), Visits 7 (Week 16), 9 (Week 26), and 10 (Week 28, 2 weeks after the last dose).

Urine samples for UACR will be obtained via first morning void on the 2 consecutive days leading up to and morning of Visits 5, 6, 8, 9 and 10. The key efficacy endpoint evaluation will take place at the End of Treatment Visit (Visit 9).

### Dosage and Administration

(R)-Compound 1 tablets, active or placebo, will be provided in blister packs. Placebo tablets will be indistinguishable from the (R)-compound 1 tablets. Two tablets per day will be self-administered orally.

Patients will be allowed their normal diet the morning of study drug administration. At the Randomization Visit (Visit 3), patients will receive either (R)-compound 1 tablets of their assigned dose strength or matching placebo tablets. Patients will self-administer the first dose of study drug in two tablets at the clinical site. Subsequent doses of the study drug are to be taken by the patient by mouth once daily (QD) at approximately the same time each morning at home. On days of clinical site visits, patients will take their scheduled morning doses of their ACEi, ARB and SGLT2 inhibitor (if applicable) at home and to hold their dose of study drug on the morning of their next visit. Patients must bring their study drug and background ACEi or ARB medications to the clinical site at all visits. At the clinical site, patients will self-administer the study drug to be witnessed by site staff after completion of pre-dose evaluations and laboratory sampling.

### Endpoints

One efficacy endpoint is the change in mean seated SBP from baseline to Week 26 of (R)-compound 1 compared to placebo. The SBP will be measured by seated automated office blood pressure monitoring (AOBPM).

Other endpoints of the study are:
The change from baseline of SBP in (R)-compound 1 at various dose groups compared to placebo at week 26 in a hierarchical order of ≥2 mg, 2 mg, 1 mg, 0.5 mg, and 4 mg;
The percentage of patients achieving SBP<130 mmHg after 26 weeks of treatment by dosing strategy;
The percentage of change from baseline in urinary albumin-to-creatinine ratio (UACR) with each dosing strategy of (R)-compound 1 compared to placebo after 26 weeks of treatment;
The change from baseline in diastolic blood pressure (DBP) measured by AOBPM with each dosing strategy of (R)-compound 1 compared to placebo at Week 26;
The change from baseline in estimated glomerular filtration rate (eGFR) after 26 weeks of treatment;
The change from baseline with each dose strength of (R)-compound 1 compared to placebo after 26 weeks of treatment in pharmacodynamic (PD) variables including, but not limited to, the following:
Serum and/or plasma parameters: electrolytes, plasma aldosterone concentration (PAC), 11-deoxycorticosterone, B-type natriuretic peptide (BNP), plasma renin activity (PRA), direct renin concentration, angiotensinogen, and angiotensin II; or
24-hour urine parameters: electrolytes, aldosterone, renin, kidney injury molecule-1 (KIM-1), cystatin C, growth differentiation factor-15 (GDF-15), neutrophil-gelatinase associated lipocalin (NGAL), transforming growth factor beta (TGF-0), and monocyte chemoattractant protein-1 (MCP1).
The relationship between percentage of change in UACR and changes in aldosterone and renin levels with each dose strength of (R)-compound 1 compared to placebo after 26 weeks of treatment;
The relationship between change in UACR and changes in blood pressure (BP) with each dose strength of (R)-compound 1 compared to placebo after 26 weeks of treatment;
The relationship between the change in eGFR and changes in BP with each dose strength of (R)-compound 1 compared to placebo after 26 weeks of treatment.

The pharmacokinetic (PK)-PD objective of the study is to evaluate the exposure-response relationships of (R)-compound 1 in patients with uncontrolled hypertension and CKD using measures of safety, PD, and/or efficacy.

### The Safety Endpoints are

Changes in vital signs, standing BP and heart rate, physical examinations, electrocardiography, weight measurement, and clinical laboratory evaluations, including standard safety chemistry panel, hematology, coagulation, and urinalysis;
Treatment-emergent adverse events (TEAEs);
Treatment-emergent adverse events of interest (AEOI)
TEAEs leading to premature discontinuation of study drug;
Treatment-emergent marked laboratory abnormalities; and
Changes in standing SBP and DBP (measured pre-dose at the clinical site).

The safety of (R)-compound 1 will be assessed from the time of randomization until the end of the Follow-Up Period. The safety endpoints will include the following:
Change from in potassium levels from baseline to Week 26 between each dose strength of (R)-compound 1 compared to placebo;
Vital signs (heart rate, respiratory rate, and body temperature), mean SBP, mean DBP, orthostatic vitals (standing BP and heart rate), physical examinations, electrocardiogram (ECG), weight measurement, and clinical laboratory evaluations including standard safety chemistry panel, hematology, coagulation, and urinalysis;
The incidence of treatment-emergent adverse events (TEAEs);
The incidence of treatment-emergent SAEs;
The incidence of TEAEs leading to premature discontinuation of the study drug;
The incidence of treatment-emergent marked laboratory abnormalities; and
The change in standing SBP and DBP (measured at the clinical site prior to administration of study drug) from baseline to Week 26;

AEs of special interest will include the following: hypotension events that require clinical intervention, abnormal potassium laboratory values that require clinical intervention, and abnormal sodium laboratory values that require clinical intervention.

### Efficacy Analysis

The efficacy analysis will compare the change in mean seated SBP from baseline to Week 26 of (R)-compound 1 and placebo. A mixed-model for repeated measures (MMRM) will be used to perform this analysis. The analysis will include fixed effects for treatment, visit, and treatment-by-visit interaction, along with a covariate of the baseline value. An estimate of the treatment difference at week 26 will be generated, as will an assessment of whether this estimate is significantly different when comparing placebo with each dosing strategy at a two-sided 0.05 level of significance. The least squares means, standard errors, and 2-sided 95% confidence intervals (CIs) for each treatment group and for pairwise comparisons of each dosing strategy of (R)-compound 1 to the placebo group will be provided.

Missing data will be imputed using multiple imputation methodology. Results will be combined using Rubin's method. Full details of the model and imputation will be provided in the SAP.

The efficacy analysis will compare the change in SBP, DBP and UACR from baseline (Visit 3) to End of Treatment (Visit 9) between each dose of (R)-compound 1 and placebo. Percentage change in UACR will be calculated by analysis of covariance using log-transformed UACR values, with baseline log-transformed UACR as a covariate. A mixed-model for repeated measures will be used to perform this analysis. The analysis will include fixed effects for treatment, visit, and treatment-by-visit interaction, along with a covariate of the baseline value. The restricted maximum likelihood estimation approach will be used with an unstructured covariance matrix. Least squares mean, standard errors, and 2-sided 95% CIs for each treatment group and for pairwise comparisons of each dosing strategy of (R)-compound 1 to the placebo group will be provided. Adjustment for multiple comparisons will be made using Dunnett's test in accordance with the power and sample size calculations utilized for the study. Missing data will be imputed using multiple imputation methodology. The results will be combined using Rubin's method. Full details of the model and imputation will be provided in the SAP.

Similar models will be used to analyze eGFR and PD variables. Logistic regression analyses will be used to analyze binary endpoints with model covariates of treatment group, baseline BP, baseline DBP, and baseline eGFR. No adjustment will be made for multiplicity in testing the efficacy endpoints.

### Safety Analysis

The Safety Population will be the population for the safety analysis. All safety endpoints will be summarized descriptively.

The assessment of safety will be based primarily on the frequency of AEs, clinical laboratory assessments, vital signs, and 12-lead ECGs. Other safety data will be summarized as appropriate.

AEs will be coded using the Medical Dictionary for Regulatory Activities. TEAEs, defined as those AEs that newly occur or worsen in severity during the Double-Blind Treatment Period, will be summarized by system organ class and preferred term. A list of patients with SAEs, AE of special interest and those who discontinue from the study due to an AE will be provided.

Summary statistics by treatment strategy group at baseline, at each visit, and of changes from baseline to each visit for laboratory parameters, vital signs, and other safety measurements will be provided. The occurrence of significant abnormalities in change from baseline of laboratory values will be summarized by treatment group. Physical examination data will be listed.

Clinical Study in Patients with Renal Impairment

Of particular relevance to study subjects who will be enrolled in this trial is the investigation of the pharmacokinetics of (R)-compound 1 in individuals with varying degrees of renal impairment. The PK profiles of (R)-compound 1 and its primary metabolite following administration of a single 10 mg dose in individuals with renal impairment were qualitatively and quantitatively similar to those measured in healthy subjects. Pair-wise comparisons of plasma PK parameters for (R)-compound 1 in the moderate to severe renal impairment group confirmed the lack of noteworthy effect across groups with geometric mean ratios of 1.02, 1.21, and 1.17 for Cₘₐₓ, AUC_{(0-inf)}, and AUC₍₀₋ₗₐₛₜ₎, respectively, as compared to the control group. Higher exposures to (R)-compound 1 were not observed in the kidney failure group as compared to the control group with geometric mean ratios of 0.88, 0.68, and 0.68 for Cₘₐₓ, AUC_{(0-inf)}, and AUC₍₀₋ₗₐₛₜ₎, respectively. The conclusions of these studies demonstrated that a single 10 mg dose of (R)-compound 1 was well tolerated when administered to individuals with varying degrees of renal function, including those with moderate to severe renal impairment or kidney failure (on hemodialysis). There were no noteworthy increases in systemic exposure or decrease in clearance in individuals with impaired renal function. Dose adjustment of (R)-compound 1 based on renal function was therefore not necessary.

The results of the renal impairment study indicated that a single 10 mg dose of (R)-compound 1 was well tolerated by subjects with varying degrees of renal function, ranging from normal renal function to end stage disease receiving hemodialysis. One subject with end stage disease experienced an unrelated SAE of metabolic encephalopathy and a moderate unrelated adverse event (AE) of tremor. One control subject experienced a mild AE of diarrhea, which was considered to be related to study drug by the Investigator. There were no clinically significant changes in laboratory values (including potassium), ECGs, or vital signs. PK data from this study demonstrated that there was no noteworthy increase in systemic exposure or decrease in renal clearance in individuals with moderate or severe renal impairment (estimated glomerular filtration rate [eGFR] 15 to 59 mL/min) as compared to control subjects (normal renal function or mild renal impairment; eGFR≥60 mL/min). Likewise, no noteworthy increase in plasma exposure to (R)-compound 1 in subjects with end stage renal disease (eGFR<15 mL/min or on hemodialysis) was observed; however, these subjects did not produce adequate urine to assess differences in renal clearance in this population. It is not necessary to dose adjust (R)-compound 1 for patients with renal impairment.

Results of the renal impairment study demonstrated that (R)-compound 1 was well tolerated when administered to individuals with varying degrees of renal function, including those with moderate to severe renal impairment or kidney failure (on hemodialysis). Overall, there were no deaths, 1 (3.0%) subject experienced an SAE, and no subjects discontinued due to a TEAE. Overall, 2 (6.1%) subjects experienced a total of 3 TEAEs: 1 (10.0%) subject in the control group experienced a TEAE of mild diarrhea following administration of (R)-compound 1 that was considered related to the study drug; no subjects experienced a TEAE in the (R)-moderate to severe renal impairment group; and 1 (8.3%) subject in the kidney failure group experienced 2 TEAEs of moderate tremor and severe metabolic encephalopathy (recorded as an SAE) following administration of (R)-compound 1 that were considered not related to the study drug. The SAE was thought to be secondary to the concomitant medications. As such, there was no apparent increase in incidence or severity of AEs with decreased renal function. There were no AEs, trends, or clinically meaningful changes in laboratory parameters. There were no AEs or clinically significant changes observed in vital signs, physical examinations, or 12-lead ECGs.

Results of the renal impairment study demonstrated that the plasma concentration-time curves of (R)-compound 1 in each renal function group were qualitatively similar to one another.

There was no substantial impact of renal impairment on the PK of (R)-compound 1 based on pair-wise comparisons and no strong or nonlinear relationship between estimated GFR and PK parameters was observed. Findings for the primary metabolite of (R)-compound 1 was similar to those for parent with no clinically meaningful differences observed across groups. The percent of the dose excreted renally was approximately 12% in the control group and the moderate to severe renal impairment group. Inadequate urine production in the kidney failure group resulted in negligible renal excretion in this group. Based on the results of this study, dose adjustment of (R)-compound 1 in renally impaired individuals is not considered necessary.

### Drug Supplies

### Formulation and Packaging

(R)-compound 1 will be provided as 0.5 mg, 1 mg, and 2 mg tablets in blister packs. (R)-compound 1 tablets will contain the active ingredient and inactive ingredients.

### Example 6

A Randomized, Double-Blind Study to Evaluate the Safety, Pharmacokinetics, and Pharmacodynamics of (R)-compound 1 Following Multiple Oral Doses in Healthy Subjects

### Study Objectives

The objectives of this study include:
To assess the safety and tolerability of (R)-compound 1 following oral dosing once daily for 10 days under normal and low salt conditions;
To characterize the pharmacokinetics (PK) of (R)-compound 1 following oral dosing once daily for 10 days under normal and low salt conditions; and
To characterize the pharmacodynamic (PD) effects of (R)-compound 1 following oral dosing once daily for 10 days under normal and low salt conditions.

### Methodology

This was a randomized, double-blind study to assess the safety, tolerability, PK, and PD of multiple oral doses of (R)-compound 1 when administered to healthy adult subjects. The study consisted of 5 cohorts, with up to 12 subjects per cohort. Subjects were randomized in a 3:1 ratio to (R)-compound 1 or placebo once daily for 10 days as follows:
Cohort 1: 2.5 mg (R)-compound 1 or matching placebo (low salt diet in 9 subjects receiving (R)-compound 1 and 3 subjects receiving placebo);
Cohort 2: 5.0 mg (R)-compound 1 or matching placebo (low salt diet in 9 subjects receiving (R)-compound 1 and 3 subjects receiving placebo);
Cohort 3: 1.5 mg (R)-compound 1 or matching placebo (normal salt diet in 9 subjects receiving (R)-compound 1 and 3 subjects receiving placebo);
Cohort 4: 2.5 mg (R)-compound 1 or matching placebo (normal salt diet in 6 subjects receiving (R)-compound 1 and 2 subjects receiving placebo); and
Cohort 5: 0.5 mg (R)-compound 1 or matching placebo (normal salt diet in 9 subjects receiving (R)-compound 1 and 3 subjects receiving placebo).

Cohorts 1 and 2 dosed concurrently, with a minimum 5-day lag between the first dose for Cohort 2 and the first dose for Cohort 1. Cohorts 3 through 5 dosed concurrently.

For each subject, the study consisted of:
A screening period of up to 28 days;
A 5-day inpatient run-in period during which subjects adhered to a controlled, standardized diet and underwent baseline PD assessments;
A 15-day inpatient treatment period, during which subjects received (R)-compound 1 or placebo once daily for 10 days while adhering to a controlled, standardized diet, followed by PK and PD sampling over an additional 5 days; and
A follow-up visit 3 days (1 day) after discharge from the clinical unit.
A cortisol stimulation test (Cohorts 1 and 2 only) and standing aldosterone assessment (all cohorts) were performed on Days 1 and 10. Serial blood samples for PK and PD were obtained prior to and at specified time points over 24 hours after dosing on Day 1 as well as prior to and at specified time points over 120 hours after dosing on Day 10. In addition, blood samples for PK were collected prior to dosing on Days 8 and 9. Urine for PK and PD measurements was collected over 24 hours starting just prior to dosing on Day 1 as well as over 120 hours starting just prior to dosing on Day 10.

Subjects were discharged from the clinic following completion of discharge procedures 5 days after the final dose of (R)-compound 1 or placebo and returned to the clinic for a follow-up visit 3 days (1 day) after discharge from the clinic to collect a PK sample and to capture adverse events (AEs) and concomitant medications.

Safety was assessed throughout the study based on AEs, physical examinations, weight measurements, electrocardiograms (ECGs), orthostatic vital sign assessments, and clinical laboratory evaluations. In order to thoroughly assess any potential effect of (R)-compound 1 on QT interval, continuous ECG recordings were also performed, starting approximately 1 hour before dosing, and continuing for approximately 24 hours after dosing on Days 1 and 10.

Unscheduled procedures or visits and/or additional follow-up may have been required for subjects with clinically significant abnormal laboratory findings, unresolved treatment-emergent adverse events (TEAEs), serious adverse events (SAEs) that required follow-up laboratories and review, and clinically significant AEs.

### Inclusion Criteria

Subjects who met all the following criteria based on screening and check-in results are eligible to participate in the study:
1. Healthy subjects between the ages of 18 and 55 years, inclusive, in good health based on medical and psychiatric history, physical examination, ECG, orthostatic vital signs, and routine laboratory tests (blood chemistry, hematology, coagulation, and urinalysis);
2. Body mass index (BMI) between 18 and 30 kg/m², inclusive;
3. Nonsmokers who had not used nicotine-containing products for at least 6 months prior to the screening visit;
4. Male subjects with female partners of childbearing potential must have agreed to use 2 medically accepted, highly effective methods of birth control from Day 1 through 90 days after the final dose of study drug;
5. Male subjects must have agreed to abstain from sperm donation from Day 1 through 90 days after administration of the final dose of study drug;
6. Female subjects with male partners must have been surgically sterile (hysterectomy and/or bilateral oophorectomy), postmenopausal for at least 1 year (with follicle-stimulating hormone in the postmenopausal range), or had agreed to use 2 medically accepted, highly effective methods of birth control from Day −14 until 60 days following the final dose of study drug.
7. Was able to understand and willing to comply with study procedures and restrictions (including confinement to the clinical unit and restrictions on physical activity, use of recreational drugs or alcohol, and medications) and was able to provide written informed consent according to institutional and regulatory guidelines.

### Exclusion Criteria

Subjects who met any of the following criteria based on screening and check-in results (or Day −4 results for cortisol stimulation test [Cohorts 1 and 2] or cortisol level [Cohorts 3 through 5]) are excluded from participation in the study:
1. Was actively participating in an experimental therapy study; received experimental therapy with a small molecule within 30 days of Day 1, or 5 half-lives, whichever was longer; or received experimental therapy with a large molecule within 90 days of Day 1, or 5 half-lives, whichever was longer;
2. Had a personal or family history of long QT syndrome, Torsades de Pointes, or other complex ventricular arrhythmias, or family history of sudden death;
3. Had a history of, or current, clinically significant arrhythmias as judged by the Investigator, including ventricular tachycardia, ventricular fibrillation, or atrial fibrillation. Subjects with minor forms of ectopy (e.g., premature atrial contractions) were not necessarily excluded;
4. Had prolonged QTcF (>450 msec) based on the average of triplicate ECGs;
5. Had seated BP higher than 150/90 mmHg or lower than 90/50 mmHg based on the average of ≥3 BP measurements;
6. Had resting heart rate higher than 100 beats per minute (bpm) or lower than 50 bpm based on the average of ≥3 BP measurements, sinus node dysfunction, or clinically significant heart block;
7. Had temperature greater than 37.6° C. (99.68° F., measured orally), and respiration rate <12 and >20 breaths/minute;
8. Had postural tachycardia (i.e., >30 bpm upon standing) or orthostatic hypotension (i.e., a fall in systolic blood pressure [SBP] of ≥20 mmHg or diastolic blood pressure of ≥10 mmHg when a person assumed a standing position);
9. Had serum potassium >the upper limit of normal of the reference range (ULN) and serum sodium <the lower limit of normal of the reference range;
10. Had aspartate aminotransferase and alanine aminotransferase values >1.2 ULN;
11. Was positive for HIV antibody, hepatitis C virus antibody, or hepatitis B surface antigen;
12. Had any other clinical laboratory values which were meaningfully outside of normal limits (based on laboratory normal range) in the opinion of the Investigator;
13. For Cohorts 1 and 2, had an abnormal cortisol stimulation test (<13.5 μg/100 mL) based on results of the test performed in the morning on Day −4. For Cohorts 3 through 5, had an abnormal cortisol level (<5 μg/dL) based on results of the test performed in the morning on Day −4;
14. Had a known history of porphyria, myopathy, or an active liver disease;
15. Had evidence or history of any clinically significant immunologic, hematologic, renal, endocrine, pulmonary, gastrointestinal, cardiovascular, musculoskeletal, hepatic, psychiatric, neurologic, or allergic disease (including clinically significant or multiple drug allergies); surgical conditions; cancer (with the exception of basal or squamous cell carcinoma of the skin and cancer that resolved or has been in remission for >5 years prior to the screening visit); or any condition that, in the Investigator's opinion, may have confounded study procedures or results, impacted subject safety, or interfered with the absorption, distribution, metabolism, or excretion of the study drug (appendectomy allowed, cholecystectomy prohibited);
16. Used any prescription medications (including topicals) or over-the-counter medications (other than occasional use of acetaminophen or nonsteroidal anti-inflammatory drugs [NSAIDs], such as ibuprofen or naproxen, according to the package insert); herbal supplements; dietary supplements; or nutraceuticals within 14 days prior to the first dose of study drug, or 5 half-lives, whichever was longer, or was unwilling to refrain from these medications until the follow-up visit. Use of over-the-counter topical medications may have been permitted in consultation with the Sponsor. In addition, medications for which 5 half-lives exceeded 14 days must have been discussed with and approved by the Sponsor prior to subject enrollment;
17. Used corticosteroids (systemic or extensive topical) within 3 months prior to dosing;
18. Had positive drug or alcohol test result or a history of alcoholism or drug abuse within 2 years prior to the first dose of study drug as defined by the Diagnostic and Statistical Manual of Mental Disorders, 4th Edition: DSM-IV;
19. Had typical consumption of ≥14 alcoholic drinks weekly. 1 drink of alcohol was equivalent to ½ pint of beer (285 mL), 1 glass of spirits (25 mL), or 1 glass of wine (125 mL);
20. Had history of or evidence of illicit drug use within the past 2 years;
21. Donated or lost ≥400 mL blood or donated blood products within 30 days prior to the first dose of study drug;
22. Had surgical procedures within 4 weeks of check-in or planned elective surgery during the study period;
23. Had any illness during the 4 weeks before check-in, unless deemed not clinically significant by the Investigator;
24. Had known allergy to any ingredient of the study drug ((R)-Compound 1 or placebo). Any history of severe allergic reaction (including drugs, food, insect bites, environmental allergens);
25. Had known intolerance to ACTH stimulation test product;
26. Had inadequate venous access;
27. Was currently undergoing treatment with weight loss medication or prior weight loss surgery (e.g., gastric bypass surgery);
28. Was pregnant, breastfeeding, or planning to become pregnant during the study; or
29. Was considered by the Investigator, after reviewing medical and psychiatric history, physical examination, and laboratory evaluation, to be unsuitable for any other reason that may have either placed the subject at increased risk during participation or interfered with the interpretation of the study outcomes.

### Duration of Treatment:

For each subject, study participation lasted up to 56 days. Treatment with (R)-compound 1 or placebo lasted for 10 days (once daily dosing).

### Investigational Product and Comparator Information:

Study subjects were dosed with (R)-compound 1 (oral drinking solution) or matching placebo. The doses of (R)-compound 1 administered were 2.5 and 5.0 mg with a low salt diet and 1.5, 2.5, and 0.5 mg with a normal salt diet.

### Pharmacokinetics:

The following plasma PK parameters were determined for (R)-compound 1 and its primary metabolite following the first dose of (R)-compound 1, as the data permitted:
Maximum observed plasma concentration on Day 1 (Cₘₐₓ, D1), observed directly from data;
Time to Cₘₐₓ, D1 (Tₘₐₓ, D1), observed directly from data; and
Area under the plasma concentration-time curve (AUC) from time 0 to 24 hours postdose (AUC0-24h).

The following plasma PK parameters were determined for (R)-compound 1 and its primary metabolite following the final dose of (R)-compound 1 on Day 10, as the data permitted:
Maximum observed plasma concentration on Day 10 (Cₘₐₓ, D10), observed directly from data;
Time to Cₘₐₓ, D10 (Tₘₐₓ, D10), observed directly from data;
AUC over a dosing interval (i.e., tau) (AUC₀₋ₜₐᵤ);
AUC from time 0 to the time of the last quantifiable plasma concentration (Clast) (AUC₀₋ₜ);
AUC from time 0 to infinity (AUC_{0-inf});
AUC extrapolated, the percent of AUC extrapolated, calculated as 100 (AUC_{0-inf}-AUC₀₋ₜ)/AUC_{0-inf};
Apparent first-order terminal elimination rate constant (λz);
Terminal phase elimination half-life (t½), calculated as ln(2)/λz;
Apparent plasma clearance (CLss/F), calculated as Dose/AUC₀₋ₜₐᵤ (only for (R)-compound 1); and
Apparent volume of distribution (Vss/F), calculated as Dose/[λz AUC₀₋ₜₐᵤ] (only for (R)-compound 1).

Steady-state accumulation ratios were reported as follows:
Accumulation ratio based on maximum observed plasma concentration (Cₘₐₓ) after the first dose and the final dose (RCₘₐₓ), calculated as Cₘₐₓ, D10/Cₘₐₓ, D1; and
Accumulation ratio based on AUC after the first dose and last dose (RAUC), calculated as AUC₀₋ₜₐᵤ/AUC0-24h.

The following urine PK parameters were determined for (R)-compound 1 and its primary metabolite following the first dose:
Cumulative amount of drug excreted in urine on Day 1 (Ae, D1);
Fraction of the dose excreted renally on Day 1 (Fe, D1), calculated as 100 Ae, D1/Dose (only for compound 1); and
Renal clearance on Day 1 (CLR, D1), calculated as Ae, D1/AUC0-24h.

The following urine PK parameters are determined for (R)-compound 1 and its primary metabolite following the final dose:
Cumulative amount of drug excreted in urine on Day 10 (Ae, D10);
Fraction of the dose excreted renally on Day 10 (Fe, D10), calculated as 100 Ae, D10/Dose (only for (R)-compound 1); and
Renal clearance on Day 10 (CLR, D10), calculated as Ae, D10/AUC_{0-inf}.
Pharmacodynamics:
Plasma PD measures included concentrations of:
Aldosterone and its precursors 18-hydroxycorticosterone, corticosterone, and 11-deoxycorticosterone.
Cortisol (free and total) and its precursor 11-deoxycortisol;
Plasma renin activity (PRA); and
Adrenocorticotropic hormone (ACTH).
Plasma electrolyte measures included concentrations of
   Sodium;
   Chloride; and
   Potassium.

For analysis, AUC values are calculated for aldosterone and cortisol (free and total) and all precursor parameters for the following time periods, when possible, using a non-compartmental method as appropriate:
Area under the PD effect-time curve from time 0 to 4 hours postdose (AUC0-4h);
Area under the PD effect-time curve from time 4 hours postdose to 12 hours postdose (AUC4-12h);
Area under the PD effect-time curve from time 0 to 12 hours postdose (AUC0-12h); and
Area under the PD effect-time curve from time 0 to 24 hours postdose (AUC0-24h).

The linear trapezoidal/linear interpolation method was used in the calculation of AUCs. Nominal time points were used in the AUC calculations.

Urine PD measures included concentrations of
Aldosterone;
Cortisol (free and total); and
Tetrahydroaldosterone.

For analysis, the urine PD concentrations were converted to total analyte amounts: Total amount (unit)=concentration (unit/volume)
urine volume

(Urine volume may have been converted from urine mass collected using 1 mg=1 mL). Urine electrolyte measures included concentrations of
Sodium;
Chloride;
Potassium;
Creatinine; and
Phosphorus.

For analysis, the urine sodium to potassium ratio was also calculated.

### Safety:

Safety is assessed throughout the study based on AEs, physical examinations, weight measurements, ECGs, vital sign (including orthostatic) assessments, and clinical laboratory evaluations. In order to thoroughly assess any potential effect of (R)-compound 1 on QT interval, continuous ECG recordings were also performed starting approximately 1 hour before dosing and continuing for approximately 24 hours after dosing on Days 1 and 10. If cardiodynamic analyses are undertaken, 12-lead ECGs will be extracted at predefined time points paired with PK samples.

### Summary of Results

Results of the MAD study indicate that multiple ascending doses of (R)-compound 1 up to 5 mg QD for 10 days were also well tolerated by healthy subjects under low salt (2.5 and 5 mg of (R)-compound 1) and normal salt conditions (0.5, 1.5, and 2.5 mg of (R)-compound 1). Specifically, there were no deaths, SAEs, or treatment-emergent adverse events (TEAEs) leading to withdrawal and there were no clinically significant changes in electrocardiograms (ECGs including no meaningful changes in QT interval corrected by Fridericia's formula [QTcF]) or vital signs. The most common TEAEs following administration of multiple doses of (R)-compound 1 were headache, postural dizziness, and dizziness. No clinically significant changes were observed over time in the safety laboratory test results. PK data from the MAD study indicate that exposure to (R)-compound 1 (as assessed based on Cₘₐₓ and AUC) is generally 2- to 2.5-fold higher at steady state as compared to that observed following a single dose. Exposures within the dose range studied increased in an approximately dose-proportional manner. PD data from this study confirmed the ability of (R)-compound 1 to lower aldosterone at doses ≤5 mg without affecting levels of cortisol or its precursor 11-deoxycortisol in healthy subjects. As expected with a reduction in aldosterone levels, there were mild, dose-dependent increases in plasma potassium levels and reduction in plasma sodium levels.

### Pharmacokinetics:

Pharmacokinetic results from the MAD study indicate that exposure to (R)-compound 1 (as assessed based on Cₘₐₓ and AUC) is generally 2- to 2.5-fold higher at steady state as compared to that observed following a single dose. Exposures within the dose range studied increase in an approximately dose-proportional manner.

After oral administration, (R)-compound 1 was rapidly absorbed with peak concentrations typically observed within 4 hours after dosing (median time to maximum observed plasma concentration [Tₘₐₓ] ranged from 0.98 to 4 hours across treatment groups). Exposures declined from peak slowly in an apparent biphasic manner, with a long mean t½ ranging from approximately 26 to 31 hours.

At steady state, exposure was typically approximately 2- to 2.5-fold higher than after a single dose (mean RCₘₐₓ values ranged from approximately 1.7 to 2.4 and mean RAUC values ranged from 2 to 2.5 across treatment groups). Though the current study was not designed or powered to formally assess dose proportionality, the data were subjected to an exploratory dose proportionality assessment and (R)-compound 1 is considered to be dose proportional over the dose range studied.

On average, approximately 7% (range of 6.3% to 10.8% across treatment groups) of the (R)-compound 1 dose was recovered unchanged in the urine.

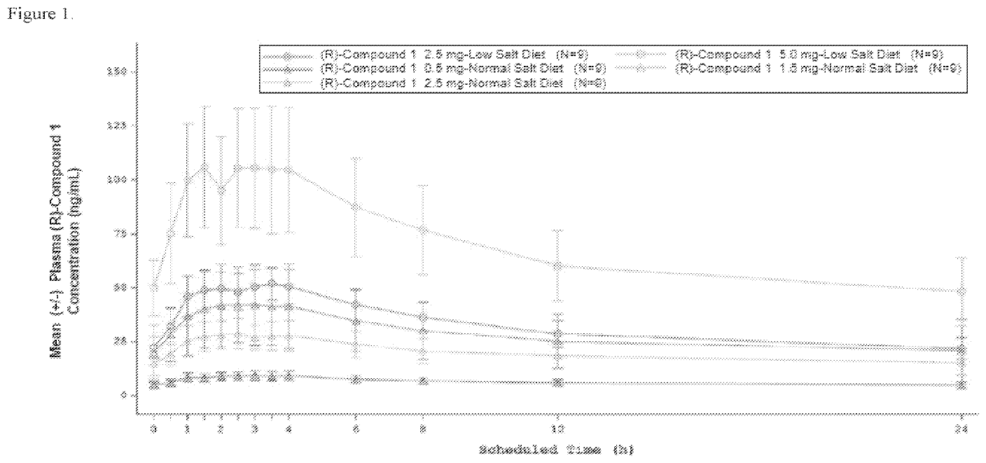

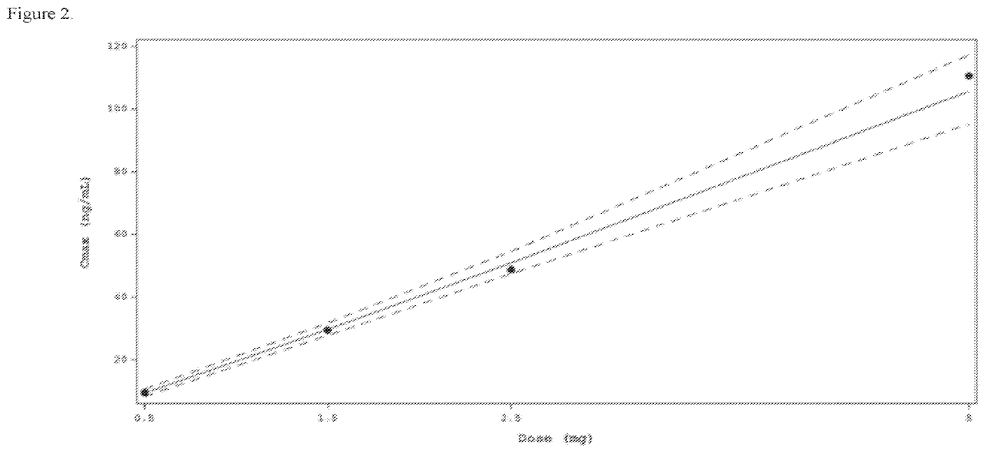

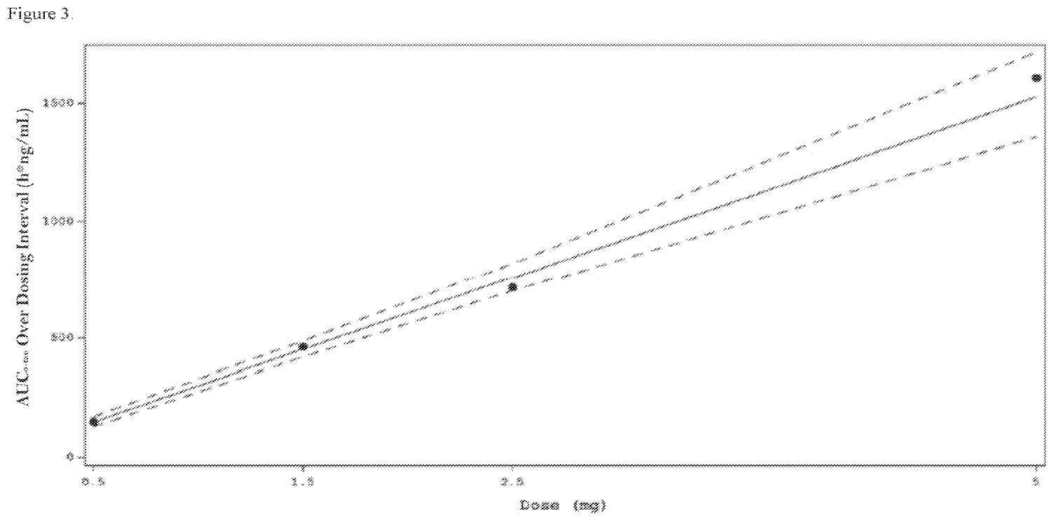

The primary metabolite of (R)-compound 1 was formed slowly over time after the initial dose of (R)-compound 1 (median Tₘₐₓ ranged from approximately 4 to 24 hours across treatment groups), with a steady-state (Day 10) median Tₘₐₓ observed within 4 hours (median Tₘₐₓ ranged from 3.5 to 4.0 hours across treatment groups). See FIG. 1. Metabolite levels increased with increasing dose. Plasma concentrations of metabolite generally declined from peak slowly, with a long mean t½ ranging from approximately 31 to 38 hours. At steady state, exposure (as assessed based on RCₘₐₓ and RAUC values) was approximately 2.4- to 3.5-fold higher than after a single dose. The metabolite represents, on average, 8.0% to 11% of parent based on Cₘₐₓ, D10 and 10% to 22% of parent based on AUC_{0-inf}. Plots of Cₘₐₓ and AUC₀₋ₜₐᵤ versus (R)-compound 1 dose (Day 10—Pharmacokinetic Population) are shown in FIGS. 2 and 3 respectively.

### Pharmacodynamics:

Marked inhibition of aldosterone synthesis was observed after the initial dose of (R)-compound 1 and sustained throughout the 10-day dosing period under both normal salt diet and low salt diet conditions; however, the effect of 0.5 mg (R)-compound 1 was similar to that of placebo.

Measurements following a subject's early termination from study drug were excluded. AUC0-12h=area under the pharmacodynamic effect-time curve from time 0 to 12 hours postdose; LS=least squares.

The effect of (R)-compound 1 on aldosterone at doses ≥1.5 mg was observed both in the presence and absence of the Cortrosyn challenge, with an approximate 70% to 85% decrease in mean AUC0-12h typically being observed as compared to baseline. Levels of the interim aldosterone precursors 18-hydroxycorticosterone and corticosterone demonstrated stepwise changes indicative of a progressive impact of cytochrome P450 11B2 inhibition on the pathway of aldosterone synthesis.

Specifically, levels of 18-hydroxycorticosterone (the immediate precursor to aldosterone) were generally comparable to or decreased from baseline but to a lesser extent than observed decreases in aldosterone. Levels of corticosterone, which is further upstream from aldosterone, increased in an apparent dose-dependent manner. Finally, levels of 11-deoxycorticosterone, the initial aldosterone precursor, showed modest (approximately 2- to 3-fold) increases in predose values as compared to baseline, with changes being most apparent under low salt diet conditions in which subjects also underwent a cortisol stimulation test.

There were no apparent effects of (R)-compound 1 on cortisol (total or free) or 11-deoxycortisol, including in the presence of Cortrosyn challenge (which occurred with the low salt diet treatment groups). Consistent with observations from the mean time course and AUC data for cortisol, (R)-compound 1 had no apparent effect on response to the Cortrosyn challenge, with Day 1 and Day 10 responses in (R)-compound 1-treated subjects being similar to their response at baseline and to the response in subjects receiving placebo.

The low salt diet conditions in Cohorts 1 and 2 resulted in an increase in ACTH. The increases were somewhat more pronounced in subjects receiving (R)-compound 1 as compared to subjects receiving placebo. Following administration of (R)-compound 1 under normal salt diet conditions, however, (R)-compound 1 resulted in apparent dose-dependent decreases in ACTH.

There were no clinically significant changes in seated vital signs or dose-related trends. There was a slight decrease in seated BP for the overall (R)-compound 1 treatment group compared to the overall pooled placebo group, although there was no clear dose dependency. Slight trends towards mild, drug-induced decreases in orthostatic BP and moderate increases in orthostatic heart rate were observed. As observed for the seated vital signs, these trends in orthostatic measurements were not consistently dose dependent. However, the most pronounced effects on heart rate were observed at the 5 mg (R)-compound 1 dose level. Changes in BP when moving from supine to standing position were smaller on Day 10 as compared to baseline in all treatment groups (compound 1 and placebo).

However, the decreases were generally larger in subjects receiving (R)-compound 1 as compared to subjects receiving placebo, particularly for systolic blood pressure (SBP). Likewise, increases in heart rate observed 1 minute after moving from a supine to a standing position were more pronounced in subjects receiving (R)-compound 1 as compared to subjects receiving placebo. Although there were no clear and consistent dose-related trends, the most pronounced changes in 1-minute standing heart rate were typically noted at the 5 mg (R)-compound 1 dose level.

Mild, dose-dependent decreases in plasma sodium levels and increases in plasma potassium levels were observed, as would be expected with a reduction in aldosterone levels. Urine sodium and potassium values corresponded to changes observed in plasma. Specifically, on Day 1 following the first dose of (R)-compound 1, the sodium:potassium ratio was increased, with the sodium loss in urine greater than the potassium retention. However, this effect had diminished by Day 10, suggesting that the balance between sodium excretion and potassium absorption was restored. The change in the sodium:potassium ratio appears to be mediated by a greater elimination of sodium in the urine on Day 1 as compared to sodium elimination in the urine on Day 10, as potassium appears not to change over the course of the 10-day treatment period.

There were mild increases in blood urea nitrogen, creatinine, and the blood urea nitrogen:creatinine ratio. A mild reduction in glomerular filtration rate (<15%) was also observed. The presence of increased blood urea nitrogen:creatinine ratio with reduced glomerular filtration rate suggests that (R)-compound 1 is producing a mild diuretic effect.

Mean body weight and BMI decreased slightly from baseline during the treatment period in all groups, including placebo, under both low salt diet and normal salt diet conditions. However, the decrease in mean body weight and BMI in subjects receiving (R)-compound 1 was more pronounced (−1.31 kg and −0.442 kg/m², respectively, in the overall (R)-compound 1 group) as compared to that in subjects receiving placebo (−0.02 kg and −0.016 kg/m², respectively, in the pooled placebo group).

There was no clear dose dependency in the observed decreases in the (R)-compound 1 treatment groups. Values generally returned to baseline at the follow-up visit.

### Safety:

Treatment with (R)-compound 1 was safe and well tolerated. There were no deaths, SAEs, or discontinuations due to a TEAE, and there were no apparent increases in incidence or severity of AEs with increasing doses. The most common System Organ Class of TEAEs was nervous system disorders. All TEAEs were mild in severity except for 1, a moderate TEAE of ventricular tachycardia. Among subjects receiving (R)-compound 1, 4 (9.5%) subjects experienced headache, 3 (7.1%) subjects experienced postural dizziness (preferred term of dizziness postural), and 2 (4.8%) subjects experienced dizziness. Generally, there were no clinically meaningful changes from baseline in laboratory parameters during the study; however, there were isolated abnormal sodium and potassium values, which were likely due to a combination of the protocol-specified dietary sodium requirements and the effects of (R)-compound 1. There were no clinically meaningful changes in physical examination results or clinically significant changes in 12-lead ECG findings during the study, including no meaningful changes in QTcF.

### Conclusions

Administration of (R)-compound 1 once daily for 10 days was safe and well tolerated by healthy subjects. (R)-compound 1 is rapidly absorbed and exhibits a long t½ conducive to once daily dosing with predictable increases in exposure over the dose range studied. Accumulation of (R)-compound 1 at steady-state is typically approximately 2- to 2.5-fold higher than after a single dose.

Treatment with (R)-compound 1 resulted in marked, sustained, selective, and generally dose dependent inhibition of aldosterone synthesis under both normal salt diet and low salt diet conditions without impact on cortisol or 11-deoxycortisol levels. The inhibition of aldosterone synthase associated with administration of (R)-compound 1 produced expected changes in aldosterone precursors, with increases observed in corticosterone and 11-deoxycorticosterone while 18-hydroxycorticosterone remained comparable or decreased.

There were no clinically significant changes in BP with (R)-compound 1 as compared to placebo.

Despite their normotensive status, there were slight reductions in SBP in subjects receiving (R)-compound 1 as compared to subjects receiving placebo, particularly upon standing. Heart rate increases upon standing were also greater following administration of (R)-compound 1 versus placebo.

Mild, dose-dependent decreases in plasma sodium levels and increases in plasma potassium levels were observed with corresponding changes in urine sodium and potassium levels. Mild increases in blood urea nitrogen:creatinine ratio and mild decreases in glomerular filtration rate were also observed following administration of (R)-compound 1.

Of the randomized subjects, 41 (97.6%) subjects in the overall (R)-Compound 1 treatment group and 13 (92.9%) subjects in the overall pooled placebo treatment group completed the study. There was 1 (2.4%) subject (Subject 001-082) in the overall (R)-Compound 1 treatment group who withdrew early from the study. This subject received 2.5 mg (R)-Compound 1 with normal salt diet and withdrew due to physician decision after steady state was disrupted pending repeat laboratory test results. There was 1 (7.1%) subject (Subject 001-101) in the overall pooled placebo treatment group who withdrew early from the study. This subject received placebo with normal salt diet and the reason for withdrawal was withdrawal by subject (the subject did not want to continue participation).

### Single Dose and Steady-State Plasma Pharmacokinetics of (R)-Compound 1

After oral administration, (R)-Compound 1 was rapidly absorbed with peak concentrations typically observed within 4 hours after dosing. Concentrations declined from peak in an apparent biphasic manner. The plasma concentration-time profile of (R)-Compound 1 over a dosing interval on Day 10 was qualitatively similar to that observed on Day 1.

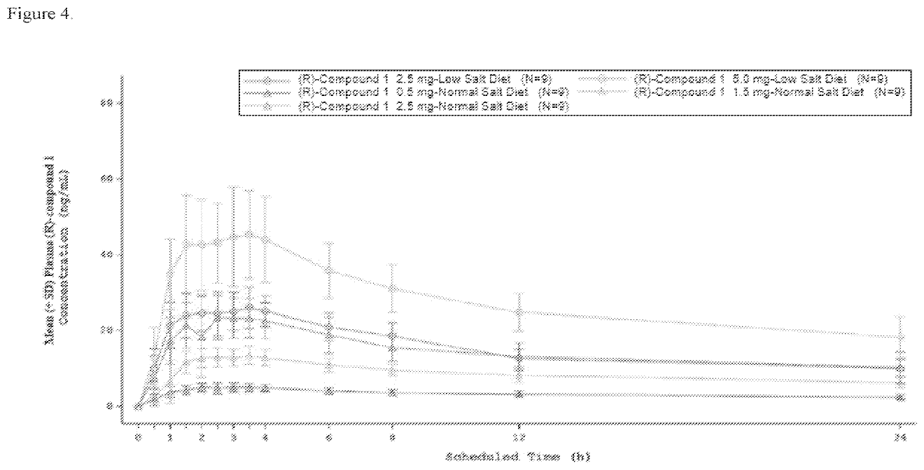

FIG. 4 displays the plot of mean (SD) plasma (R)-Compound 1 concentrations versus time by treatment for all (R)-Compound 1 treatment groups over 24 hours following the first dose of (R)-Compound 1 (Day 1) on a linear scale for the PK Population.

Single Dose (Day 1) and Steady-State (Day 10) Plasma Pharmacokinetic Parameters of (R)-Compound 1 are summarized in Table 1. Mean (% CV) Pharmacokinetic Parameters are summarized in Tables 2 and 3.

TABLE 1
Summary of Single Dose (Day 1) and Steady-State (Day 10) Plasma Pharmacokinetic
Parameters of (R)-Compound 1 - Pharmacokinetic Evaluable Population

| Day | | Normal Salt Diet | | | Low Salt Diet | |
|---|---|---|---|---|---|---|
| Urine PK | | 0.5 mg | 1.5 mg | 2.5 mg | 2.5 mg | 5.0 mg |
| Parameter | Statistic | (R)-Compound 1 | (R)-Compound 1 | (R)-Compound 1 | (R)-Compound 1 | (R)-Compound 1 |

Day 1

| | | | | | | |
|---|---|---|---|---|---|---|
| A_{e, D 1} (ng) | n | 9 | 9 | 6 | 9 | 9 |
| | Mean (CV %) | 39952.889 | 115762.122 | 270541.533 | 1687233.672 | 317339.306 |
| | | (42.0) | (33.2) | (29.4) | (33.2) | (29.1) |
| F_{e, D 1} (%) | n | 9 | 9 | 6 | 9 | 9 |
| | Mean (CV %) | 7.991 | 7.717 | 10.822 | 6.749 | 6.347 |
| | | (42.0) | (33.2) | (29.4) | (33.2) | (29.1) |
| CL_{R, D 1} (mL/h) | n | 9 | 9 | 6 | 9 | 9 |
| | Mean (CV %) | 0.533 | 0.575 | 0.818 | 0.478 | 0.489 |
| | | (48.4) | (35.2) | (28.3) | (37.3) | (26.3) |

Day 10

| | | | | | | |
|---|---|---|---|---|---|---|
| A_{e, D 10} (ng) | n | 9 | 9 | 5 | 9 | 9 |
| | Mean (CV %) | 168181.917 | 459968.526 | 826482.355 | 766791.190 | 1677304.469 |
| | | (56.6) | (54.2) | (42.3) | (33.1) | (36.6) |
| F_{e, D 10} (%) | n | 9 | 9 | 5 | 9 | 9 |
| | Mean (CV %) | 33.636 | 30.665 | 33.059 | 30.672 | 33.546 |
| | | (56.6) | (54.2) | (42.3) | (33.1) | (36.6) |
| CL_{R, D 10} (mL/h) | n | 9 | 9 | 5 | 8 | 9 |
| | Mean (CV %) | 0.493 | 0.440 | 0.668 | 0.510 | 0.493 |
| | | (46.9) | (43.2) | (30.7) | (19.3) | (27.5) |

A_{e, D1} = cumulative amount of drug excreted in urine on Day 1; A_{e, D 10} = cumulative amount of drug excreted in urine on Day 10; CL_{R, D1} = renal clearance on Day 1; CL_{R, D 10} = renal clearance on Day 10; CV = coefficient of variability; F_{e, D 1} = fraction of the dose excreted renally on Day 1; F_{e, D 10} = fraction of the dose excreted renally on Day 10; PK = pharmacokinetic(s).

TABLE 2
Mean (% CV) Pharmacokinetic Parameters of (R)-compound 1 Following Table 2.

| | 1 mg | | 3 mg | | 10 mg | | 30 mg | | 90 mg | | 180 mg | | 360 mg | |
|---|---|---|---|---|---|---|---|---|---|---|---|---|---|---|
| Parameter | (n = 9) | | (n = 9) | | (n = 9) | | (n = 6) | | (n = 6) | | (n = 6) | | (n = 6) | |
| Cₘₐₓ (ng/ml) | 10.1 | (12) | 31.9 | (14) | 125 | (12) | 386 | (20) | 1380 | (19) | 2490 | (16) | 5750 | (7) |
| Tₘₐₓ (h)^{a} | 1 | (0.5-3) | 2 | (0.5-4) | 0.5 | (0.5-1) | 0.75 | (0.5-3) | 1.25 | (0.5-4) | 1 | (0.5-4) | 0.5 | (0.5-1) |
| AUC₀₋₂₄ | 147 | (15) | 491 | (11) | 1602 | (9) | 5262 | (18) | 18322 | (11) | 33241 | (16) | 73225 | (10) |
| (ng · h/mL) | | | | | | | | | | | | | | |
| AUC_{0-inf} | 330 | (26) | 1070 | (14) | 3630 | (17) | 11000 | (18) | 32200 | (13) | 65800 | (28) | 147000 | (14) |
| (ng · h/mL) | | | | | | | | | | | | | | |
| t_{1/2} (h) | 27.4 | (19) | 29.1 | (19) | 30.7 | (16) | 29.4 | (11) | 25.3 | (19) | 28.0 | (16) | 27.8 | (4) |
| CL/F (mL/h) | 3270 | (32) | 2820 | (14) | 2820 | (17) | 2820 | (20) | 2830 | (13) | 2900 | (24) | 2480 | (13) |

| | | | | | | | |
|---|---|---|---|---|---|---|---|
| AUC_{extrapoloated} | 1.66 | 1.68 | 1.37 | 0.986 | 0456 | 0.789 | 0719 |
| (%) | | | | | | | |
| Vz/F (mL) | 122000 | 117000 | 123000 | 11800 | 102000 | 114000 | 994 |

AUC0₋₂₄ = area under the plasma concentration versus time curve from 0 to 24 hours postdose; AUC0_{-inf} = area under the plasma concentration versus time curve extrapolated to infinity; AUC_{extrapolated} = percent of area under the plasma concentration-time curve extrapolated; CL/F = apparent oral clearance; Cₘₐₓ = maximum observed plasma concentration; CV = coefficient of variation; n = number of subjects per dose group; t_{1/2} = apparent terminal half-life; Tₘₐₓ = time to maximum observed plasma concentration; Vz/F = apparent volume of distribution.
^{a}Tₘₐₓ is reported as median (range).

TABLE 3
Mean (% CV) Pharmacokinetic Parameters of (R)-compound 1 Following Single
Oral Dose and at Steady-State (Normal and/or Low Salt Conditions)

| | | Normal Salt Diet | | | Low Salt Diet | |
|---|---|---|---|---|---|---|
| | | 0.5 mg | 1.5 mg | 2.5 mg | 2.5 mg | 5.0 mg |
| Day | | (R)- | (R)- | (R)- | (R)- | (R)- |
| Plasma PK | | compound | compound | compound | compound | compound |
| Parameter | Statistic | 1 | 1 | 1 | 1 | 1 |

Day 1

| | | | | | | |
|---|---|---|---|---|---|---|
| Cₘₐₓ, D 1 | N | 9 | 9 | 6 | 9 | 9 |
| (ng/mL) | Mean (CV %) | 5.363 (23.3) | 14.03 (16.0) | 26.18 (18.2) | 28.09 (21.0) | 47.33 (26.0) |
| Tₘₐₓ, D 1 (h) | N | 9 | 9 | 6 | 9 | 9 |
| | Median | 2.0 (1.0, 4.0) | 2.50 (1.0, 4.0) | 3.00 (2.0, 4.0) | 3.00 (0.98, 4.00) | 3.02 (1.50, 4.00) |
| | (min, max) | | | | | |
| AUC_{0-24 h} | N | 9 | 9 | 6 | 9 | 9 |
| (h · ng/mL) | Mean (CV %) | 79.465 (21.8) | 205.305 (17.9) | 343.980 (26.9) | 365.790 (17.9) | 657.852 (20.9) |

Day 10

| | | | | | | |
|---|---|---|---|---|---|---|
| Cₘₐₓ, D 10 | N | 9 | 9 | 5 | 9 | 9 |
| (ng/ml) | Mean (CV %) | 9.724 (23.3) | 29.94 (20.4) | 43.88 (42.6) | 53.96 (14.0) | 113.44 (22.8) |
| Tₘₐₓ, | N | 9 | 9 | 5 | 9 | 9 |
| D 10 (h) | Median | 2.00 (1.0, 3.5) | 2.50 (1.0, 4.0) | 3.00 (2.5, 4.0) | 2.00 (0.98, 4.00) | 3.00 (1.5, 4.0) |
| | (min, max) | | | | | |
| AUC₀₋ₜₐᵤ | N | 9 | 9 | 5 | 9 | 9 |
| (h · ng/ml) | Mean (CV %) | 155.185 (23.0) | 479.668 (28.8) | 676.512 (50.8) | 782.991 (18.3) | 1659.405 (26.5) |
| AUC_{0-inf} | N | 9 | 9 | 5 | 8 | 9 |
| (h · ng/ml) | Mean (CV %) | 350.528 (27.7) | 1108.623 (47.3) | 1459.854 (79.2) | 1488.318 (18.5) | 3480.485 (35.7) |
| AUC extrap | N | 9 | 9 | 5 | 8 | 9 |
| (%) | Mean (CV %) | 6.325 (42.2) | 6.106 (65.9) | 3.832 (85.6) | 4.519 (45.4) | 5.397 (52.6) |
| t½ (h) | N | 9 | 9 | 5 | 8 | 9 |
| | Mean (CV %) | 31.160 (18.6) | 29.922 (25.8) | 25.548 (23.3) | 28.370 (16.8) | 29.364 (22.0) |
| CLss/F (L/h) | N | 9 | 9 | 5 | 9 | 9 |
| | Mean (CV %) | 3.421 (29.4) | 3.350 (27.5) | 4.315 (36.9) | 3.277 (16.1) | 3.207 (26.3) |
| Vss/F (L) | N | 9 | 9 | 5 | 9 | 9 |
| | Mean (CV %) | 151.579 (29.4) | 136.871 (11.5) | 150.377 (28.2) | 134.699 (27.2) | 134.266 (31.3) |
| RCₘₐₓ | N | 9 | 9 | 5 | 9 | 9 |
| | Mean (CV %) | 1.827 (14.2) | 2.132 (11.2) | 1.706 (34.1) | 1.965 (15.1) | 2.424 (13.6) |
| RAUC | N | 9 | 9 | 5 | 9 | 9 |
| | Mean (CV %) | 1.960 (13.9) | 2.307 (13.8) | 1.994 (22.3) | 2.147 (7.4) | 2.507 (10.2) |

For subjects whose apparent first-order terminal elimination rate constant was not assigned based on Statistical Analysis Plan rules (i.e., the adjusted regression coefficient (R-squared) was less than 0.8), the AUC0-24 was calculated using observed concentrations at 24 hour postdose.
AUC0-24 = area under the plasma concentration-time curve from time 0 to 24 hours postdose; AUC_{0-inf} = area under the plasma concentration-time curve from time 0 to infinity; AUC₀₋ₜₐᵤ = area under the plasma concentration-time curve over a dosing interval; AUC extrap = percent of area under the plasma concentration-time curve extrapolated; CLss/F = apparent plasma clearance; Cₘₐₓ, D 1 = maximum observed plasma concentration on Day 1; Cₘₐₓ, D 10 = maximum observed plasma concentration on Day 10; CV = coefficient of variability; max = maximum; min = minimum; PK = pharmacokinetic(s); RAUC = accumulation ratio based on the area under the plasma concentration-time curve after the first dose and the last dose; RCₘₐₓ = accumulation ratio based on the maximum observed plasma concentration after first dose and the final dose; t½ = terminal phase elimination half-life; TmaxD 1 = time to maximum observed plasma concentration on Day 1; Tmax, D 10 = time to maximum observed plasma concentration on Day 10; Vss/F = apparent volume of distribution.

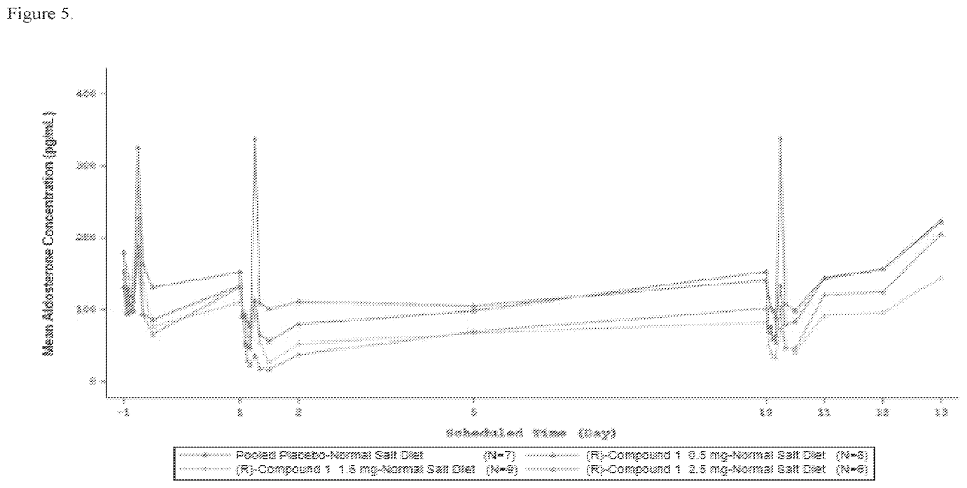

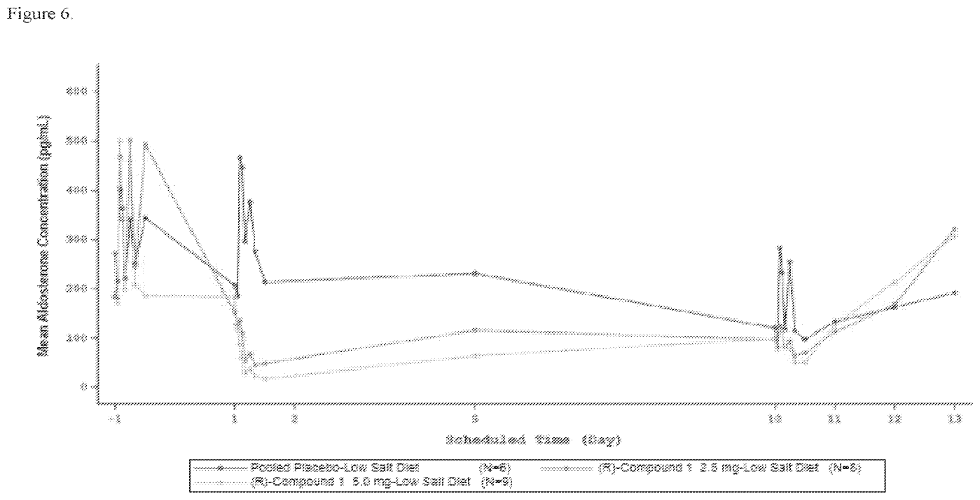

Plots of mean aldosterone plasma concentration over time by treatment for normal salt and low salt diet treatment groups are shown in FIGS. 5 and 6, and summarized in Tables 4 and 5.

TABLE 4
Plasma Aldosterone Concentration and Percent Change From Day −1 to
Day 10 - Pharmacodynamic Population (Excluding Outlier Subjects)

| | | (R)-Compound 1 | | | | | Pooled Placebo | |
|---|---|---|---|---|---|---|---|---|
| Parameter | | Normal Salt Diet | | | Low Salt Diet | | Normal | Low Salt |
| (Unit)/Visit | Statistic | 0.5 mg | 1.5 mg | 2.5 mg | 2.5 mg | 5.0 mg | Salt Diet | Diet |

Aldosterone (pg/mL)

| | | | | | | | | |
|---|---|---|---|---|---|---|---|---|
| Day −1 predose | n | 8 | 9 | 6 | 8 | 9 | 7 | 6 |
| | Mean | 131.588 | 143.100 | 153.667 | 272.125 | 189.633 | 179.700 | 183.883 |
| | SD | 60.6915 | 68.7665 | 40.2972 | 147.0835 | 121.1750 | 150.4071 | 127.9403 |
| Day 1, predose | n | 8 | 9 | 6 | 8 | 9 | 7 | 6 |
| | Mean | 133.013 | 110.567 | 131.250 | 151.988 | 182.800 | 152.614 | 206.867 |
| | SD | 59.5566 | 21.6652 | 57.2623 | 72.5843 | 86.2694 | 115.9945 | 166.1079 |
| Day 1, 12 hours | n | 8 | 9 | 6 | 8 | 9 | 7 | 6 |
| postdose | Mean | 56.250 | 27.111 | 16.535 | 48.013 | 17.511 | 101.329 | 214.150 |
| | SD | 36.3810 | 16.3896 | 12.4140 | 20.1895 | 7.1464 | 106.9761 | 215.0410 |
| Day 2, predose | n | 8 | 9 | 6 | NA | NA | 7 | NA |
| | Mean | 80.100 | 52.933 | 37.700 | NA | NA | 111.457 | NA |
| | SD | 30.7760 | 28.2778 | 16.2412 | NA | NA | 72.6705 | NA |
| Day 10, predose | n | 8 | 9 | 5 | 8 | 9 | 6 | 6 |
| | Mean | 152.875 | 82.144 | 101.880 | 97.538 | 98.367 | 141.300 | 120.417 |
| | SD | 73.7436 | 42.8040 | 48.5355 | 42.3058 | 21.2772 | 69.9021 | 64.0456 |
| Day 10, 12 hours | n | 8 | 9 | 5 | 8 | 9 | 6 | 6 |
| postdose | Mean | 83.625 | 41.300 | 46.360 | 69.100 | 49.467 | 99.033 | 97.317 |
| | SD | 56.3319 | 17.6874 | 10.8999 | 33.3925 | 18.9425 | 34.7845 | 59.9481 |
| Day 10, 24 hours | n | 8 | 9 | 5 | 8 | 9 | 6 | 6 |
| postdose | Mean | 143.338 | 92.533 | 120.740 | 112.213 | 125.811 | 144.800 | 133.183 |
| | SD | 74.8884 | 51.0080 | 62.4657 | 46.3130 | 27.6997 | 86.1330 | 77.7270 |
| Percent change | n | 8 | 9 | 5 | 8 | 9 | 6 | 6 |
| from Day −1 to | Mean | 28.841 | −37.405 | −34.296 | −56.540 | −36.400 | 14.968 | −23.344 |
| Day 10, predose | SD | 69.0031 | 34.3229 | 32.6191 | 28.6921 | 23.4522 | 125.4815 | 42.6628 |

Change and percent change from baseline are relative to time-matched Day −1 time points. Below the limit of quantitation values were imputed to lower limit of quantitation values for analysis use.
NA = not available; SD = standard deviation.

TABLE 5
Urine Aldosterone and Tetrahydroaldosterone Percent Change From Baseline (Day −1)
to Day 1 and Day 10 - Pharmacodynamic Population (Excluding Outlier Subjects)

| | | (R)-Compound 1 | | | | | Pooled Placebo | |
|---|---|---|---|---|---|---|---|---|
| Parameter | | Normal Salt Diet | | | Low Salt Diet | | Normal | Low Salt |
| (Unit)/Visit | Statistic | 0.5 mg | 1.5 mg | 2.5 mg | 2.5 mg | 5.0 mg | Salt Diet | Diet |

Aldosterone (ng)

| | | | | | | | | |
|---|---|---|---|---|---|---|---|---|
| Percent change | n | 8 | 9 | 6 | 8 | 9 | 7 | 6 |
| from baseline | | | | | | | | |
| (Day −1) to | Mean | −23.7290 | −67.6229 | −71.7141 | −61.1877 | −83.5768 | 21.4501 | 13.0016 |
| Day 1 0-24 hr | SD | 22.77591 | 8.20330 | 8.15860 | 44.52869 | 2.52822 | 54.03918 | 25.75507 |
| Percent change | n | 8 | 9 | 5 | 8 | 9 | 6 | 6 |
| from baseline | Mean | 4.2728 | −65.6831 | −50.7592 | −19.3985 | −68−9282 | 0.4809 | −31.7615 |
| (Day −1) to | | | | | | | | |
| Day 10 0-24 hr | SD | 67.53371 | 11.53699 | 6.59786 | 164.94270 | 8.51415 | 43.24180 | 38.18828 |

Tetrahydroaldosterone (ng)

| | | | | | | | | |
|---|---|---|---|---|---|---|---|---|
| Percent change | n | 8 | 9 | 6 | 8 | 9 | 7 | 6 |
| from baseline | Mean | −12.0171 | −47.0556 | −59.6517 | −26.0540 | −67.5905 | 2.8460 | 6.4238 |
| (Day −1) to | | | | | | | | |
| Day 1 0-24 hr | SD | 29.17470 | 8.68890 | 15.49878 | 105.16393 | 7.48454 | 3.41450 | 14.08131 |
| Percent change | n | 8 | 9 | 5 | 8 | 9 | 6 | 6 |
| from baseline | Mean | 11.0511 | −60.9034 | −55.1771 | −6.2967 | −64.5164 | −16.3095 | −29.3527 |
| (Day −1) to | | | | | | | | |
| Day 10 0-24 hr | SD | 72.50815 | 8.55617 | 18.10705 | \|181.88861 | 9.68927 | 35.54405 | 36.32771 |

Below limit quantitation values were imputed to lower limit of quantitation values for analysis use.
Measurements following a subject's early termination from study drug were excluded.
SD = standard deviation.

Plasma 11-Deoxycorticosterone concentrations over time by treatment for normal salt and low salt diet treatment groups are shown in Tables 6 to 9.

TABLE 6
Plasma 11-Deoxycorticosterone Concentration and Percent Change From Day
−1 to Day 10 - Pharmacodynamic Population (Excluding Outlier Subjects)

| | | (R)-Compound 1 | | | | | Pooled Placebo | |
|---|---|---|---|---|---|---|---|---|
| Parameter | | Normal Salt Diet | | | Low Salt Diet | | Normal | Low Salt |
| (Unit)/Visit | Statistic | 0.5 mg | 1.5 mg | 2.5 mg | 2.5 mg | 5.0 mg | Salt Diet | Diet |

11-Deoxycorticosterone (pg/mL)

| | | | | | | | | |
|---|---|---|---|---|---|---|---|---|
| Day −1 predose | n | 8 | 9 | 6 | 8 | 9 | 7 | 6 |
| | Mean | 52.625 | 43.489 | 50.017 | 72.688 | 49.111 | 54.557 | 49.467 |
| | SD | 40.7394 | 23.4208 | 17.9084 | 80.2038 | 39.4448 | 41.8702 | 36.9183 |
| Day 1, predose | n | 8 | 9 | 6 | 8 | 9 | 7 | 6 |
| | Mean | 50.938 | 44.711 | 53.200 | 83.825 | 55.789 | 51.514 | 66.650 |
| | SD | 19.7869 | 16.6626 | 29.2047 | 50.9480 | 22.4610 | 25.2290 | 45.3892 |
| Day 1, 12 hours | n | 8 | 9 | 6 | 8 | 9 | 7 | 6 |
| postdose | Mean | 35.900 | 19.656 | 23.667 | 133.300 | 94.622 | 15.819 | 59.133 |
| | SD | 16.6491 | 6.5240 | 8.3660 | 86.7581 | 41.3200 | 4.4835 | 33.1469 |
| Day 2, predose | n | 8 | 9 | 6 | NA | NA | 7 | NA |
| | Mean | 72.950 | 53.344 | 69.617 | NA | NA | 71.814 | NA |
| | SD | 48.6458 | 24.7120 | 30.2684 | NA | NA | 66.7361 | NA |
| Day 10, predose | n | 8 | 9 | 5 | 8 | 9 | 6 | 6 |
| | Mean | 112.375 | 73.322 | 130.680 | 164.725 | 218.611 | 56.217 | 28.500 |
| | SD | 73.8458 | 33.5219 | 80.9717 | 118.4331 | 98.5773 | 21.8190 | 8.2455 |
| Day 10, 12 hours | n | 8 | 9 | 5 | 8 | 9 | 6 | 6 |
| postdose | Mean | 70.813 | 46.733 | 93.780 | 204.250 | 219.700 | 25.550 | 21.367 |
| | SD | 47.9099 | 26.2272 | 70.1740 | 147.2821 | 99.3377 | 15.2320 | 5.6031 |
| Day 10, 24 hours | n | 8 | 9 | 5 | 8 | 9 | 6 | 6 |
| postdose | Mean | 115.663 | 84.767 | 177.400 | 217.975 | 308.444 | 74.233 | 26.933 |
| | SD | 75.1023 | 39.5078 | 139.8193 | 154.0686 | 138.3122 | 70.7427 | 6.1494 |
| Percent change | n | 8 | 9 | 5 | 8 | 9 | 6 | 6 |
| from Day −1 to | Mean | 161.115 | 104.945 | 171.567 | 412.560 | 573.144 | 88.334 | −21.042 |
| Day 10, predose | SD | 208.7129 | 123.0281 | 211.6021 | 729.9861 | 506.7111 | 216.4666 | 43.2896 |

Change and percent change from baseline are relative to time-matched Day −1 time points. Below the limit of quantitation values were imputed to lower limit of quantitation values for analysis use.
NA = not available; SD = standard deviation.

TABLE 7
Predose Plasma Corticosterone and 18-Hydroxycorticosterone Concentration and Percent
Change From Day −1 to Day 10 - Pharmacodynamic Population (Excluding Outlier Subjects)

| | | (R)-Compound 1 | | | | | Pooled Placebo | |
|---|---|---|---|---|---|---|---|---|
| Parameter | | Normal Salt Diet | | | Low Salt Diet | | Normal | Low Salt |
| (Unit)/Visit | Statistic | 0.5 mg | 1.5 mg | 2.5 mg | 2.5 mg | 5.0 mg | Salt Diet | Diet |

Corticosterone (pg/mL)

| | | | | | | | | |
|---|---|---|---|---|---|---|---|---|
| Day −1 predose | n | 8 | 9 | 6 | 8 | 9 | 7 | 6 |
| | Mean | 4302.5 | 3748.3 | 6395.0 | 6383.8 | 3700.7 | 3484.7 | 5675.0 |
| | SD | 6017.73 | 2011.69 | 4568.39 | 9417.27 | 3183.65 | 3339.17 | 3499.03 |
| Day 1, predose | n | 8 | 9 | 6 | 8 | 9 | 7 | 6 |
| | Mean | 3170.0 | 3988.3 | 5828.3 | 3711.5 | 2726.3 | 3290.0 | 6321.7 |
| | SD | 1918.50 | 2081.52 | 4445.29 | 2549.04 | 1228.25 | 2018.60 | 5197.38 |
| Day 1, 12 hours | n | 8 | 9 | 6 | 8 | 9 | 7 | 6 |
| postdose | Mean | 695.6 | 635.2 | 889.0 | 1653.4 | 890.3 | 424.4 | 659.3 |
| | SD | 282.30 | 303.89 | 447.88 | 1165.83 | 367.81 | 144.02 | 265.49 |
| Day 2, predose | n | 8 | 9 | 6 | NA | NA | 7 | NA |
| | Mean | 4490.0 | 4413.3 | 6328.3 | NA | NA | 4797.1 | NA |
| | SD | 5638.60 | 1459.64 | 2685.68 | NA | NA | 5437.21 | NA |
| Day 10, predose | n | 8 | 9 | 5 | 8 | 9 | 6 | 6 |
| | Mean | 4187.5 | 5677.8 | 8102.0 | 7190.0 | 11962.2 | 3185.0 | 4810.0 |
| | SD | 2630.86 | 2678.57 | 1895.35 | 3491.79 | 3724.79 | 1209.72 | 3745.85 |
| Day 10, 12 hours | n | 8 | 9 | 5 | 8 | 9 | 6 | 6 |
| postdose | Mean | 1211.4 | 1417.0 | 2704.0 | 3412.5 | 4738.9 | 1011.8 | 350.2 |
| | SD | 820.60 | 904.41 | 1897.39 | 2329.30 | 2879.04 | 1109.57 | 170.11 |
| Day 10, 24 hours | n | 8 | 9 | 5 | 8 | 9 | 6 | 6 |
| postdose | Mean | 5923.8 | 6065.6 | 11182.0 | 10012.5 | 13976.7 | 5163.3 | 3155.0 |
| | SD | 4966.27 | 2397.98 | 6062.78 | 8230.46 | 4464.87 | 5141.01 | 1913.74 |
| Percent change | n | 8 | 9 | 5 | 8 | 9 | 6 | 6 |
| from Day −1 to | Mean | 73.0 | 115.1 | 60.6 | 132.0 | 460.2 | 64.7 | −9.6 |
| Day 10, predose | SD | 136.09 | 179.04 | 79.86 | 225.60 | 409.98 | 200.68 | 51.17 |

18-hydroxycorticosterone (pg/mL)

| | | | | | | | | |
|---|---|---|---|---|---|---|---|---|
| Day −1 predose | n | 8 | 9 | 6 | 8 | 9 | 7 | 6 |
| | Mean | 955.5 | 1027.4 | 1185.8 | 1269.6 | 966.3 | 1026.4 | 1133.5 |
| | SD | 255.66 | 325.15 | 182.00 | 757.83 | 419.63 | 384.85 | 211.10 |
| Day 1, predose | n | 8 | 9 | 6 | 8 | 9 | 7 | 6 |
| | Mean | 943.1 | 991.7 | 1087.0 | 839.6 | 825.8 | 1001.1 | 1009.2 |
| | SD | 120.91 | 326.45 | 240.38 | 232.02 | 272.81 | 246.13 | 345.14 |
| Day 1, 12 hours | n | 8 | 9 | 6 | 8 | 9 | 7 | 6 |
| postdose | Mean | 310.1 | 225.6 | 267.3 | 230.4 | 162.3 | 377.0 | 563.3 |
| | SD | 119.67 | 44.26 | 108.38 | 101.38 | 43.66 | 182.32 | 352.02 |
| Day 2, predose | n | 8 | 9 | 6 | NA | NA | 7 | NA |
| | Mean | 819.6 | 829.3 | 901.2 | NA | NA | 955.6 | NA |
| | SD | 156.62 | 88.61 | 140.17 | NA | NA | 264.64 | NA |
| Day 10, predose | n | 8 | 9 | 5 | 8 | 9 | 6 | 6 |
| | Mean | 983.1 | 1047.9 | 1073.4 | 895.4 | 1159.6 | 983.7 | 947.8 |
| | SD | 237.82 | 269.93 | 94.27 | 241.15 | 154.96 | 204.65 | 253.25 |

TABLE 8
Predose Plasma Corticosterone and 18-Hydroxycorticosterone Concentration
and Percent Change From Day −1 to Day 10 - Pharmacodynamic
Population (Excluding Outlier Subjects) (Continued)

| | | (R)-Compound 1 | | | | | Pooled Placebo | |
|---|---|---|---|---|---|---|---|---|
| Parameter | | Normal Salt Diet | | | Low Salt Diet | | Normal | Low Salt |
| (Unit)/Visit | Statistic | 0.5 mg | 1.5 mg | 2.5 mg | 2.5 mg | 5.0 mg | Salt Diet | Diet |

18-hydroxycorticosterone (pg/mL)

| | | | | | | | | |
|---|---|---|---|---|---|---|---|---|
| Day 10, 12 hours | n | 8 | 9 | 5 | 8 | 9 | 6 | 6 |
| postdose | Mean | 326.5 | 318.4 | 345.6 | 339.1 | 405.2 | 436.3 | 297.0 |
| | SD | 150.39 | 40.35 | 72.93 | 123.83 | 141.39 | 147.08 | 89.36 |
| Day 10, 24 hours | n | 8 | 9 | 5 | 8 | 9 | 6 | 6 |
| postdose | Mean | 1016.4 | 1000.1 | 1238.0 | 961.8 | 1298.0 | 975.7 | 895.7 |
| | SD | 231.32 | 205.74 | 222.87 | 385.21 | 251.47 | 263.89 | 268.91 |
| Percent change | n | 8 | 9 | 5 | 8 | 9 | 6 | 6 |
| from Day −1 to | Mean | 5.2 | 6.1 | −10.4 | −22.0 | 38.2 | −2.7 | −15.6 |
| Day 10 | SD | 23.48 | 26.23 | 18.32 | 18.51 | 55.62 | 27.37 | 19.71 |

Change and percent change from baseline are relative to time-matched Day −1 time points. Below the limit of quantitation values were imputed to lower limit of quantitation values for analysis use.
NA = not available; SD = standard deviation.

TABLE 9
Plasma Total Cortisol Concentration and Percent Change From Day −1 to
Day 10 - Pharmacodynamic Population (Excluding Outlier Subjects)

| | | (R)-Compound 1 | | | | | Pooled Placebo | |
|---|---|---|---|---|---|---|---|---|
| Parameter | | Normal Salt Diet | | | Low Salt Diet | | Normal | Low Salt |
| (Unit)/Visit | Statistic | 0.5 mg | 1.5 mg | 2.5 mg | 2.5 mg | 5.0 mg | Salt Diet | Diet |

Total cortisol (ng/mL)

| | | | | | | | | |
|---|---|---|---|---|---|---|---|---|
| Day −1 predose | n | 8 | 9 | 6 | 8 | 9 | 7 | 6 |
| | Mean | 86.74 | 90.79 | 107.50 | 90.39 | 83.17 | 97.49 | 111.50 |
| | SD | 27.587 | 28.316 | 22.443 | 21.648 | 35.524 | 35.131 | 28.390 |
| Day 1, predose | n | 8 | 9 | 6 | 8 | 9 | 7 | 6 |
| | Mean | 84.15 | 97.61 | 106.85 | 71.75 | 68.24 | 100.10 | 100.52 |
| | SD | 12.423 | 27.655 | 42.299 | 16.901 | 24.432 | 33.069 | 42.722 |
| Day 1, 12 hours | n | 8 | 9 | 6 | 8 | 9 | 7 | 6 |
| postdose | Mean | 28.14 | 25.32 | 30.10 | 21.90 | 19.43 | 32.67 | 34.45 |
| | SD | 10.129 | 6.562 | 9.045 | 5.950 | 6.145 | 10.727 | 10.615 |
| Day 2, predose | n | 8 | 9 | 6 | NA | NA | 7 | NA |
| | Mean | 88.33 | 95.12 | 109.23 | NA | NA | 110.67 | NA |
| | SD | 28.253 | 19.425 | 24.944 | NA | NA | 43.027 | NA |
| Day 10, predose | n | 8 | 9 | 5 | 8 | 9 | 6 | 6 |
| | Mean | 81.90 | 96.54 | 106.52 | 91.85 | 112.67 | 91.45 | 104.33 |
| | SD | 23.977 | 15.455 | 16.276 | 24.263 | 21.311 | 13.623 | 23.670 |
| Day 10, 12 hours | n | 8 | 9 | 5 | 8 | 9 | 6 | 6 |
| postdose | Mean | 22.36 | 41.31 | 31.86 | 22.93 | 31.90 | 39.53 | 22.80 |
| | SD | 8.779 | 32.948 | 11.679 | 4.144 | 11.855 | 20.192 | 2.961 |
| Day 10, 24 hours | n | 8 | 9 | 5 | 8 | 9 | 6 | 6 |
| postdose | Mean | 95.76 | 90.27 | 126.20 | 94.36 | 121.40 | 114.62 | 94.95 |
| | SD | 32.344 | 23.007 | 16.947 | 30.306 | 23.871 | 40.011 | 17.481 |
| Percent change | n | 8 | 9 | 5 | 8 | 9 | 6 | 6 |
| from Day −1 to | Mean | −2.83 | 19.44 | −0.09 | 5.29 | 58.40 | 10.04 | −3.51 |
| Day 10, predose | SD | 26.297 | 54.860 | 13.092 | 32.749 | 73.415 | 72.092 | 24.639 |

Change and percent change from baseline are relative to time-matched Day −1 time points. Below the limit of quantitation values were imputed to lower limit of quantitation values for analysis use.
NA = not available; SD = standard deviation.

Table 10 presents mean predose plasma ACTH concentrations over time and change and percent change from Day 1 to Day 10 predose concentrations by treatment group for the PD Population. Low salt diet conditions resulted in an increase in ACTH. The increases were somewhat more pronounced in subjects receiving (R)-compound 1 as compared to subjects receiving placebo. Following administration of (R)-compound 1 under normal salt conditions, however, (R)-compound 1 resulted in apparent dose-dependent decreases in ACTH.

TABLE 10
Predose Plasma Adrenocorticotropic Hormone Concentration and Change and Percent Change
From Day 1 to Day 10 - Pharmacodynamic Population (Excluding Outlier Subjects)

| | | (R)-Compound 1 | | | | | Pooled Placebo | |
|---|---|---|---|---|---|---|---|---|
| Parameter | | Normal Salt Diet | | | Low Salt Diet | | Normal | Low Salt |
| (Unit)/Visit | Statistic | 0.5 mg | 1.5 mg | 2.5 mg | 2.5 mg | 5.0 mg | Salt Diet | Diet |

ACTH (pmol/L)

| | | | | | | | | |
|---|---|---|---|---|---|---|---|---|
| Day 1, predose | n | 8 | 9 | 6 | 8 | 9 | 7 | 6 |
| | Mean | 3.91 | 6.30 | 6.40 | 1.47 | 1.96 | 5.07 | 3.08 |
| | SD | 1.504 | 2.999 | 5.974 | 0.938 | 1.186 | 1.818 | 1.094 |
| Day 10, predose | n | 8 | 9 | 5 | 8 | 9 | 6 | 6 |
| | Mean | 3.76 | 4.67 | 2.80 | 3.59 | 3.52 | 5.95 | 5.47 |
| | SD | 1.351 | 2.352 | 0.718 | 1.934 | 1.545 | 2.864 | 2.715 |
| Change from | n | 8 | 9 | 5 | 8 | 9 | 6 | 6 |
| Day 1 to Day 10, | Mean | −0.15 | −1.63 | −4.04 | 2.11 | 1.57 | 0.73 | 2.38 |
| predose | SD | 0.578 | 0.966 | 6.171 | 1.286 | 1.329 | 2.330 | 1.923 |
| Percent change | n | 8 | 9 | 5 | 8 | 9 | 6 | 6 |
| from Day 1 to Day | Mean | −2.01 | −24.00 | −35.69 | 184.44 | 125.86 | 16.30 | 74.94 |
| 10, predose | SD | 17.841 | 14.520 | 40.058 | 131.485 | 165.344 | 49.744 | 52.728 |

ACTH = adrenocorticotropic hormone; SD = standard deviation.

### Pharmacokinetic and Pharmacodynamic Conclusions

(R)-Compound 1 is rapidly absorbed and exhibits a long t½ conducive to once daily dosing with predictable t½ increases in exposure over the dose range studied. Accumulation of (R)-Compound 1 at steady state is typically approximately 2- to 2.5-fold.

Treatment with (R)-Compound 1 resulted in marked, sustained, selective, and generally dose dependent inhibition of aldosterone synthesis under both normal salt diet and low salt diet conditions without impact on cortisol or 11-deoxycortisol levels. The inhibition of aldosterone synthase associated with administration of (R)-Compound 1 produced expected changes in aldosterone precursors, with increases observed in corticosterone and 11 deoxycorticosterone while 18-hydroxycorticosterone remained comparable or decreased.

There were no clinically significant changes in BP with (R)-Compound 1 as compared to placebo.

Consistent with what would be expected with an aldosterone synthase inhibitor, mild, dose-dependent decreases in plasma sodium levels and increases in plasma potassium levels were observed with corresponding changes in urine sodium and potassium levels. Mild increases in blood urea nitrogen:creatinine ratio and mild decreases in glomerular filtration rate were also observed following administration of (R)-Compound 1.

### Brief Summary of Adverse Events

There were no deaths, SAEs, or discontinuations due to a TEAE. In total, 11 (26.2%) subjects receiving (R)-Compound 1 and 3 (21.4%) subjects receiving placebo experienced a TEAE. Nearly all TEAEs were mild in severity, with 1 subject receiving placebo under low salt diet conditions experiencing a moderate TEAE (ventricular tachycardia). No subjects experienced a severe TEAE.

Overall, 6 (14.3%) subjects receiving (R)-Compound 1 and 3 (21.4%) subjects receiving placebo experienced a TEAE that was considered related to study drug by the Investigator. Nearly all study drug-related TEAEs were mild in severity, with 1 subject receiving placebo under low salt diet conditions experiencing a moderate study drug-related TEAE (ventricular tachycardia). No subjects experienced a severe study drug related TEAE.

Table 11 provides an overview of AEs by treatment at onset for the Safety Population.

TABLE 11
Overall Summary of Adverse Events by Treatment at Onset - Safety Population

| | Normal Salt Diet | | | Low Salt Diet |
|---|---|---|---|---|
| | (R)-compound 1 | (R)-compound 1 | (R)-compound 1 | (R)-compound 1 |
| | 0.5 mg | 1.5 mg | 2.5 mg | 2.5 mg |
| | (N = 9) | (N = 9) | (N = 6) | (N = 9) |
| Category | n (%) | n (%) | n (%) | n (%) |
| Subjects with any AE | 1 (11.1) | 1 (11.1) | 3 (50.0) | 3 (33.3) |
| Subjects with any TEAE | 1 (11.1) | 1 (11.1) | 3 (50.0) | 3 (33.3) |

Subjects with any TEAE by maximum severity

| | | | | |
|---|---|---|---|---|
| Mild | 1 (11.1) | 1 (11.1) | 3 (50.0) | 3 (33.3) |
| Moderate | 0 (0.0) | 0 (0.0) | 0 (0.0) | 0 (0.0) |
| Severe | 0 (0.0) | 0 (0.0) | 0 (0.0) | 0 (0.0) |
| Subjects with any study | 1 (11.1) | 0 (0.0) | 2 (33.3) | 2 (22.2) |
| drug-related TEAE | | | | |

Subjects with any study drug-related TEAE by maximum severity

| | | | | |
|---|---|---|---|---|
| Mild | 1 (11.1) | 0 (0.0) | 2 (33.3) | 2 (22.2) |
| Moderate | 0 (0.0) | 0 (0.0) | 0 (0.0) | 0 (0.0) |
| Severe | 0 (0.0) | 0 (0.0) | 0 (0.0) | 0 (0.0) |
| Subjects with any | 0 (0.0) | 0 (0.0) | 0 (0.0) | 0 (0.0) |
| treatment-emergent SAE | | | | |
| Subjects with any study | 0 (0.0) | 0 (0.0) | 0 (0.0) | 0 (0.0) |
| drug-related treatment- | | | | |
| emergent SAE | | | | |
| Subjects with any TEAE | 0 (0.0) | 0 (0.0) | 0 (0.0) | 0 (0.0) |
| leading to death | | | | |
| Subjects with any TEAE | 0 (0.0) | 0 (0.0) | 0 (0.0) | 0 (0.0) |
| leading to discontinuation | | | | |
| Subjects with any study | 0 (0.0) | 0 (0.0) | 0 (0.0) | 0 (0.0) |
| drug-related TEAE leading | | | | |
| to discontinuation | | | | |

| | | | Normal | Low Salt | |
|---|---|---|---|---|---|
| | Low Salt Diet | | Salt Diet | Diet | Overall |
| | (R)-compound 1 | Overall | Pooled | Pooled | Pooled |
| | 5.0 mg | (R)-compound 1 | Placebo | Placebo | Placebo |
| | (N = 9) | (N = 42) | (N = 8) | (N = 6) | (N = 14) |
| Category | n (%) | n (%) | n (%) | n (%) | n (%) |
| Subjects with any AE | 3 (33.3) | 11 (26.2) | 1 (12.5) | 2 (33.3 | 3 (21.4) |
| Subjects with any TEAE | 3 (33.3) | 11 (26.2) | 1 (12.5) | 2 (33.3 | 3 (21.4) |

Subjects with any TEAE by maximum severity

| | | | | | |
|---|---|---|---|---|---|
| Mild | 3 (33.3) | 11 (26.2) | 1 (12.5) | 1 (16.7) | 2 (14.3) |
| Moderate | 0 (0.0) | 0 (0.0) | 0 (0.0) | 1 (16.7) | 1 (7.1) |
| Severe | 0 (0.0) | 0 (0.0) | 0 (0.0) | 0 (0.0) | 0 (0.0) |
| Subjects with any study | 1 (11.1) | 6 (14.3) | 1 (12.5) | 2 (33.3) | 3 (21.4) |
| drug-related TEAE | | | | | |

Subjects with any study drug-related TEAE by maximum severity

| | | | | | |
|---|---|---|---|---|---|
| Mild | 1 (11.1) | 6 (14.3) | 1 (12.5) | 1 (16.7) | 2 (14.3) |
| Moderate | 0 (0.0) | 0 (0.0) | 0 (0.0) | 1 (16.7) | 1 (7.1) |
| Severe | 0 (0.0) | 0 (0.0) | 0 (0.0) | 0 (0.0) | 0 (0.0) |
| Subjects with any | 0 (0.0) | 0 (0.0) | 0 (0.0) | 0 (0.0) | 0 (0.0) |
| treatment-emergent SAE | | | | | |
| Subjects with any study | 0 (0.0) | 0 (0.0) | 0 (0.0) | 0 (0.0) | 0 (0.0) |
| drug-related treatment- | | | | | |
| emergent SAE | | | | | |
| Subjects with any TEAE | 0 (0.0) | 0 (0.0) | 0 (0.0) | 0 (0.0) | 0 (0.0) |
| leading to death | | | | | |
| Subjects with any TEAE | 0 (0.0) | 0 (0.0) | 0 (0.0) | 0 (0.0) | 0 (0.0) |
| leading to discontinuation | | | | | |
| Subjects with any study | 0 (0.0) | 0 (0.0) | 0 (0.0) | 0 (0.0) | 0 (0.0) |
| drug-related TEAE leading | | | | | |
| to discontinuation | | | | | |

TEAEs were defined as any AE, regardless of relationship to study drug, which began after the first dose of study drug. Percentage (%) was calculated as 100 x n/N.
AE = adverse event; SAE = serious adverse event; TEAE = treatment-emergent adverse event.

The most common SOC of TEAEs was nervous system disorders. Among subjects receiving (R)-Compound 1, 4 (9.5%) subjects experienced headache (1 [11.1%] subject each in the 2.5 mg (R)-Compound 1 low salt diet treatment group, 5.0 mg (R)-Compound 1 low salt diet treatment group, and 0.5 mg (R)-Compound 1 normal salt diet treatment group, and 1 [16.7%] subject in the 2.5 mg (R)-Compound 1 normal salt diet treatment group); 3 (7.1%) subjects experienced postural dizziness (PT term of dizziness postural) (1 [11.1%] subject in the 2.5 mg (R)-Compound 1 low salt diet treatment group and 2 [33.3%] subjects in the 2.5 mg (R)-Compound 1 normal salt diet treatment group); and 2 (4.8%) subjects experienced dizziness (1 [11.1%] subject in the 5.0 mg (R)-Compound 1 low salt diet treatment group and 1 [16.7%] subject in the 2.5 mg (R)-Compound 1 normal salt diet treatment group).

All other TEAEs experienced by subjects receiving (R)-Compound 1 were experienced by 1 subject each: presyncope (2.5 mg (R)-Compound 1 low salt diet treatment group), eye irritation (2.5 mg (R)-Compound 1 normal salt diet treatment group), abdominal pain (5.0 mg (R)-Compound 1 low salt diet treatment group), constipation (1.5 mg (R)-Compound 1 normal salt diet treatment group), viral infection (2.5 mg (R)-Compound 1 low salt diet treatment group), rhinitis (2.5 mg (R)-Compound 1 normal salt diet treatment group), back pain (5.0 mg (R)-Compound 1 low salt diet treatment group), anxiety (2.5 mg (R)-Compound 1 normal salt diet treatment group), dry throat (2.5 mg (R)-Compound 1 normal salt diet treatment group), and dysphonia (5.0 mg (R)-Compound 1 low salt diet treatment group).

Among subjects receiving placebo, 1 (12.5%) subject (normal salt diet) experienced palpitations, 1 (16.7%) subject (low salt diet) experienced ventricular tachycardia, and 1 (16.7%) subject (low salt diet) experienced nausea.

### Safety

Treatment with (R)-Compound 1 was safe and well tolerated. There were no deaths, SAEs, or discontinuations due to a TEAE, and there were no apparent increases in incidence or severity of AEs with increasing doses. The majority of TEAEs were mild in severity. There were no clinically meaningful changes from baseline in laboratory parameters during the study and no clinically meaningful changes in physical examination results or clinically significant changes in 12-lead ECG findings during the study, including no meaningful changes in QTcF.

### Discussion

The study was conducted in an ascending manner with interim data reviews to ensure subject safety as dose levels increased, and the dose levels studied were intended to characterize the safety, PK, and PD of (R)-Compound 1 over a meaningful dose range. The duration of dosing was intended to achieve steady-state PK and PD effects. Subjects in the initial 2 cohorts were on a low salt diet in order to stimulate aldosterone levels and to enable the evaluation of safety in a potential patient who may be following a low salt diet. Subjects in these cohorts also underwent ACTH challenge, which increases both aldosterone and cortisol levels, allowing for evaluation of the specificity of (R)-Compound 1 for aldosterone synthase. Subsequent cohorts at equal or lower doses (Cohorts 3 through 5) were on a normal salt diet.

Administration of (R)-Compound 1 once daily for 10 days was safe and well tolerated by healthy subjects. There were no deaths, SAEs, or discontinuations due to a TEAE, and there were no apparent increases in incidence or severity of AEs with increasing doses. The most common SOC of TEAEs was nervous system disorders. Among subjects receiving (R)-Compound 1, 4 (9.5%) subjects experienced headache, 3 (7.1%) subjects experienced postural dizziness (PT of dizziness postural), and 2 (4.8%) subjects experienced dizziness. All TEAEs were mild in severity, except for 1 (7.1%) subject in the overall pooled placebo group who received placebo under low salt diet conditions who experienced a moderate TEAE of ventricular tachycardia that was considered study drug related. Generally, there were no clinically meaningful changes from baseline in laboratory parameters during the study; however, there were isolated abnormal sodium and potassium values likely due to a combination of protocol-specified dietary sodium requirements and the effects of (R)-Compound 1. There were no clinically meaningful changes in physical examination results or clinically significant changes in 12-lead ECG findings during the study, including no meaningful changes in QTcF.

Consistent with the findings of the prior SAD study, levels of (R)-Compound 1 increased with increasing dose in an apparently dose-proportional manner over the dose range studied. After oral administration, (R)-Compound 1 was rapidly absorbed with peak concentrations typically observed within 4 hours after dosing. Exposures declined from peak slowly in an apparent biphasic manner, with a long mean t½ ranging from approximately 26 to 31 hours, and, at steady state, exposure was ½ typically approximately 2- to 2.5-fold higher than after a single dose. On average, approximately 7% (range of 6.3% to 10.8% across treatment groups) of the (R)-Compound 1 dose was recovered unchanged in the urine. Levels of (R)-Compound 1-M, the primary metabolite of (R)-Compound 1, increased with increasing dose and represented, on average, 8.0% to 11% of parent based on Cₘₐₓ and 10% to 22% of parent based on AUC_{0-inf} at steady state. After the initial dose of (R)-Compound 1, peak primary metabolite concentrations were attained more slowly (median Tmax ranged from approximately 4 to 24 hours across treatment groups) as compared to steady state when peak primary metabolite concentrations were observed within 4 hours. Similar to parent, plasma concentrations of (primary metabolite generally declined from peak slowly, with a long mean t½ ranging from approximately 31 to 38 hours. At steady state, exposure (as assessed based on RCₘₐₓ and R_{AUC} values) was approximately 2.4- to 3.5-fold higher than after a single dose.

The PD results of this study confirm the ability of (R)-Compound 1 to markedly decrease aldosterone levels under both low salt diet and normal salt diet conditions at doses >0.5 mg, with (R)-Compound 1 inducing a dose-dependent blunting of plasma aldosterone levels as compared to baseline and as compared to placebo. The effects were apparent after the initial dose and were sustained throughout the 10-day dosing period. The effect of (R)-Compound 1 on aldosterone at doses ≥1.5 mg was observed both in the presence and absence of the Cortrosyn challenge, with an approximate 70% to 85% decrease in mean AUC0-12h typically being observed as compared to baseline. Levels of the interim aldosterone precursors 18-hydroxycorticosterone and corticosterone demonstrated stepwise changes indicative of a progressive impact of CYP11B2 inhibition on the pathway of aldosterone synthesis. Specifically, levels of 18-hydroxycorticosterone (the immediate precursor to aldosterone) were generally comparable to or decreased from baseline but to a lesser extent than observed decreases in aldosterone. Levels of corticosterone, which is further upstream from aldosterone, increased in an Apparent dose-dependent manner. Finally, levels of 11-deoxycorticosterone, the initial aldosterone precursor, showed modest increases in predose values as compared to baseline, with changes being most apparent under low salt diet conditions in which subjects also underwent a cortisol stimulation test. The extent of the increase in predose 11 deoxycorticosterone levels, however, was minimal (2- to 3-fold) compared to what has previously been observed with another aldosterone synthase inhibitor (LCI1699) where predose 11-deoxycorticosterone levels increased up to 10-fold.

The results of this study indicate that selectivity for aldosterone synthase was maintained with repeat dosing since levels of cortisol and 11-deoxycortisol (which are solely mediated by CYP11B1) remained unaffected by (R)-Compound 1 indicating no undesired effects of (R)-Compound 1 on these steroids, even in the presence of ACTH stimulation.

The low salt diet conditions (Cohorts 1 and 2) resulted in an increase in ACTH. The increases were somewhat more pronounced in subjects receiving (R)-Compound 1 as compared to subjects receiving placebo. Following administration of (R)-Compound 1 under normal salt diet conditions, however, (R)-Compound 1 resulted in apparent dose-dependent decreases in ACTH. In contrast, the aldosterone reductions that were observed with LCI1699 were consistently associated with a corresponding increase in ACTH.

Changes in relevant laboratory measures as well as body weight and BMI were consistent with what would be expected in the presence of an aldosterone synthase inhibitor. Mild, dose dependent decreases in plasma sodium levels and increases in plasma potassium levels were observed with corresponding changes in urine sodium and potassium levels. Of note, despite an initial increase in the urine sodium:potassium ratio, indicating that the sodium loss in urine is greater than the potassium retention, the ratio normalized by Day 10, suggesting that the balance between sodium excretion and potassium absorption was restored. The change in the sodium:potassium ratio appears to be mediated by a greater elimination of sodium in the urine on Day 1 as compared to sodium elimination in the urine on Day 10, as potassium appears not to change over the course of the 10-day treatment period.

Consistent with observations for other RAAS-modifying agents, increases in blood urea nitrogen and creatinine were also observed following administration of (R)-Compound 1 along with a mild reduction (<15%) in glomerular filtration rate. The presence of an increased blood urea nitrogen:creatinine ratio with reduced glomerular filtration rate suggests that suggests that (R)-Compound 1 is producing a mild diuretic effect. Finally, subjects receiving (R)-Compound 1 experienced more pronounced decreases in body weight and BMI as compared to subjects receiving placebo, likely due to the previously noted mild diuretic effects. Values generally returned to baseline at the follow-up visit.

Although the study was conducted in healthy subjects where substantial changes in BP are not likely to be detected, the results of this study did show some slight trends towards mild, study drug-induced decreases in seated BP and orthostatic BP following administration of (R)-Compound 1 as compared to placebo, although there was no clear dose dependency in the (R)-Compound 1-related changes. Furthermore, changes in BP when moving from supine to standing position were smaller on Day 10 as compared to baseline in all treatment groups ((R)-Compound 1 and placebo). However, the decreases were generally larger in subjects receiving (R)-Compound 1 as compared to subjects receiving placebo, particularly for SBP. Likewise, increases in heart rate observed 1 minute after moving from a supine to a standing position were more pronounced in subjects receiving (R)-Compound 1 as compared to subjects receiving placebo. Although there were no clear and consistent dose-related trends, the most pronounced changes in 1-minute standing heart rate were typically noted at the 5 mg (R)-Compound 1 dose level.

Taken together, the results of the current study indicate that (R)-Compound 1 continues to represent a promising once daily treatment for the negative health impact of elevated aldosterone. The molecule has demonstrated a favorable safety profile, desirable and predictable PK attributes, and PD characteristics suggestive of potential beneficial effects in the intended patient populations at doses >0.5 mg per day.

### Pharmacokinetics

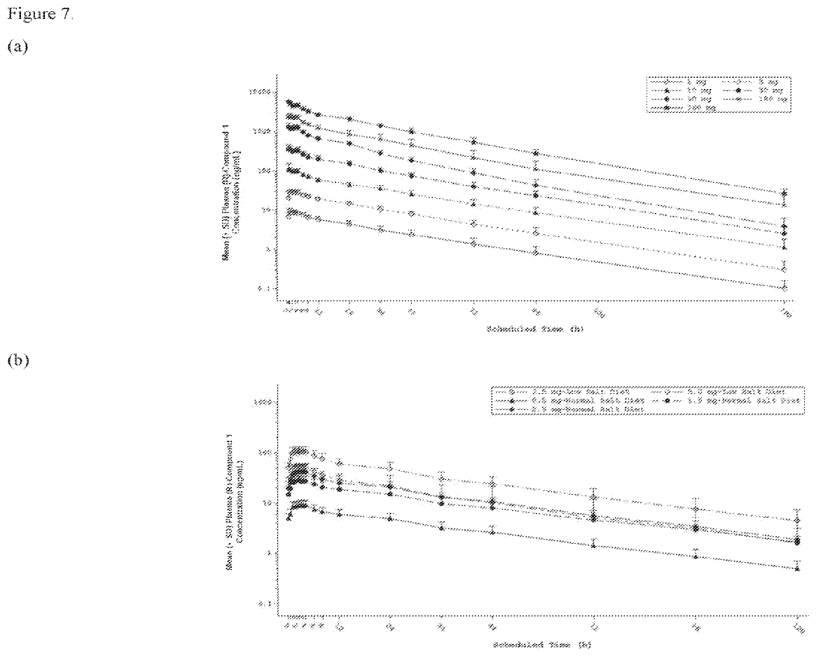

Single- and Multiple-Dose Plasma Pharmacokinetics of (R)-compound 1. The single- and multiple-dose PK profiles of (R)-compound 1 have been characterized in healthy subjects in the Phase 1 studies. FIG. 7 depicts the mean plasma concentration versus time profiles of (R)-compound 1 after administration of single oral doses of (R)-compound 1 ranging from 1 mg to 360 mg (under low salt conditions) and at steady-state over the range of 0.5 mg to 5 mg (under low and/or normal salt conditions). After oral administration, (R)-compound 1 is rapidly absorbed with peak concentrations observed within 3 hours after dosing (range 0.5 to 4 hours). (R)-compound 1 concentrations decline from peak in an apparent biphasic manner with a long mean t½ ranging from approximately 26 to 31 hours.

### Absolute Bioavailability

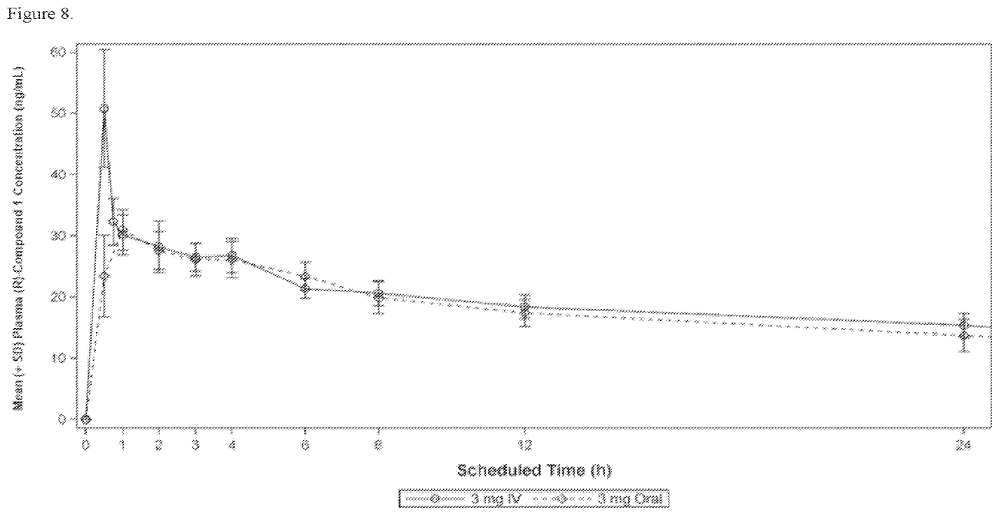

Plasma concentration versus time profiles of (R)-compound 1 following IV administration of a 3 mg dose are presented in FIG. 8. An absolute bioavailability of 97.9% was determined for (R)-compound 1 at 3 mg.

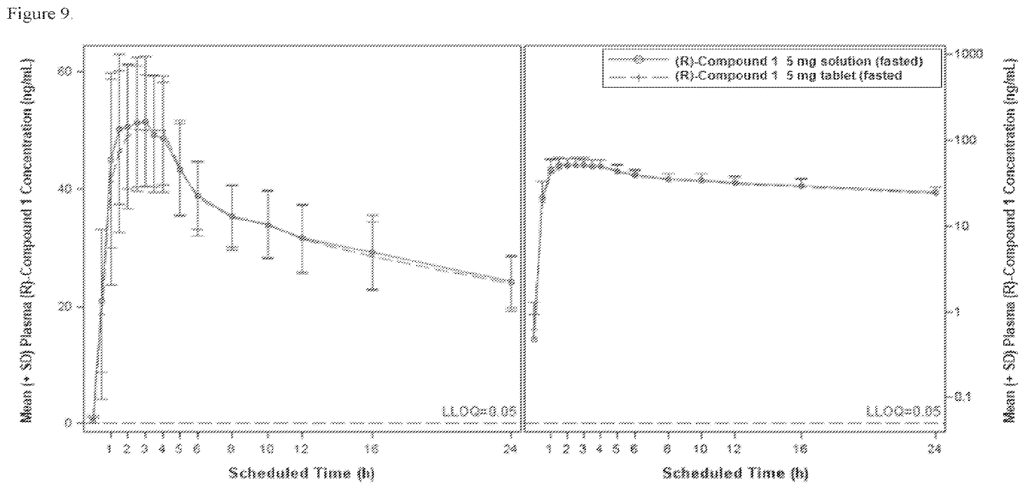

The relative bioavailability of the tablet formulation intended for use in future clinical studies as compared to the oral solution of (R)-compound 1 administered in the SAD and MAD was assessed following a single 5 mg dose of each formulation. Mean (+SD [standard deviation]) plasma concentration-time profiles following administration of the oral solution and tablet formulations are presented in FIG. 9.

Mean (SD) PK parameters of (R)-compound 1 following administration of the oral solution and tablet formulations are presented in Table 12.

TABLE 12
Summary and Analysis of Pharmacokinetic Parameters
for the (R)-compound 1 Oral Solution and Tablet

| | | 5 mg (R)-compound 1 | |
|---|---|---|---|
| PK Parameter | Statistic | Oral Solution | Tablet |
| Cₘₐₓ (ng/mL) | N | 14 | 14 |
| | Mean (SD) | 53.886 (11.6813) | 53.136 (9.7104) |
| | GM (Geo. CV %) | 52.727 (21.9) | 52.310 (18.6) |
| | Geo. LS Mean [1] | 52.12 | 52.76 |
| | Ratio of Geo. LS Means | 101.2 | |
| | (tablet/solution) | | |
| | 90% CI for Ratio [2] (%) | 95.40, 107.41 | |
| AUC₀₋ₜ | N | 14 | 14 |
| (ng · h/mL) | Mean (SD) | 1648.853 | 1618.128 |
| | | (371.9217) | (384.1689) |
| | GM (Geo. CV %) | 1610.025 (23.1) | 1577.696 (23.5) |
| | Geo. LS Mean [1] | 1584.82 | 1620.23 |
| | Ratio of Geo. LS Means | 102.2 | |
| | (tablet/solution) | | |
| | 90% CI for Ratio [2] (%) | 99.10, 105.47 | |
| AUC_{0-inf} | N | 14 | 14 |
| (ng · h/mL) | Mean (SD) | 1752.676 | 1721.792 |
| | | (425.2480) | (444.8871) |
| | GM (Geo. CV %) | 1705.336 (24.8) | 1671.781 (25.4) |
| | Geo. LS Mean [1] | 1681.33 | 1718.54 |
| | Ratio of Geo. LS Means | 102.2 | |
| | (tablet/solution) | | |
| | 90% CI for Ratio [2] (%) | 98.95, 105.59 | |
| AUCextrapolated | N | 14 | 14 |
| (%) | Mean (SD) | 5.565 (2.2131) | 5.600 (2.3669) |
| | GM (Geo. CV %) | 5.018 (56.4) | 4.995 (59.7) |
| Tmax (h) | N | 14 | 14 |
| | Median (min, max) | 2.500 (1.00, 3.50) | 3.000 (0.50, 4.07) |

Note 1:
Geo. CV % = 100 × (exp[SD²]−1)^{0.5}, where SD was the SD of the logarithm-transformed data.
Note 2:
The ANOVA model includes treatment, sequence, and period as fixed effects and subjects nested within sequence as a random effect. A subject must have a calculable PK parameter for both treatments in order to be included in the analysis for that parameter.
[1] Geometric LS Means were the LS means from the mixed model presented after back transformation to the original scale.
[2] The 90% CIs are presented after back transformation to the original scale.
ANOVA = analysis of variance;
AUC_{0-inf} = area under the plasma concentration-time curve from time 0 to infinity;
AUC₀₋ₜ = area under the plasma concentration-time curve from time 0 to the time of the last quantifiable concentration;
AUCextrapolated = percent of area under the plasma concentration-time curve extrapolated;
CI = confidence interval;
Cₘₐₓ = maximum observed plasma concentration;
CV = coefficient of variation;
Geo. = geometric;
GM = geometric mean;
h = hours;
LS = least squares;
max = maximum;
min = minimum;
PK = pharmacokinetic(s);
SD = standard deviation;
Tmax = time to maximum plasma concentration.

### Aldosterone and Precursors

Single Dose with Cortrosyn Challenge

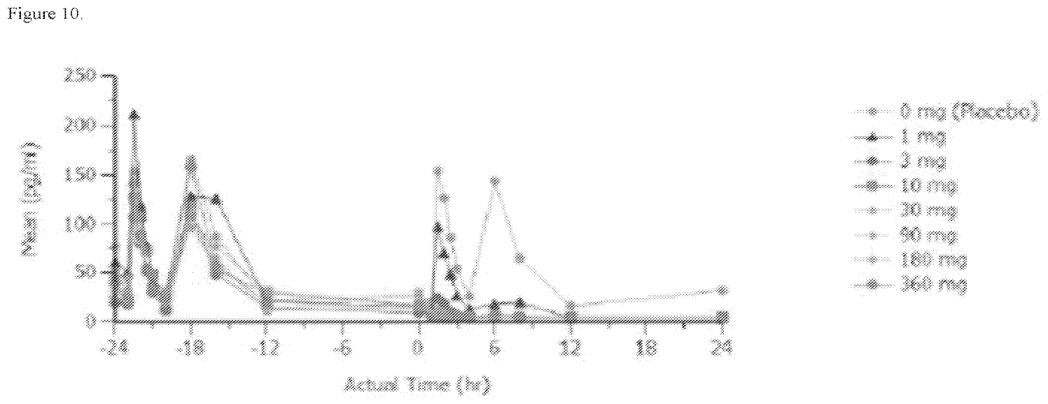

(R)-compound 1 induced a dose-dependent blunting of plasma aldosterone levels as compared to Day −1 baseline and as compared to placebo, with a maximum effect achieved at the 10 mg dose level (approximate 85% to 90% decrease as compared to Day −1). This effect was observed both on the post-Cortrosyn challenge readout (time interval 0 to 4 hours), and on the standing aldosterone peak (time interval 4 to 12 hours) (See FIG. 10).

Notes: Plasma aldosterone levels that were Below limit of quantification were set to the lower limit of quantitation value (5 pg/mL) to allow further calculation.

Cortrosyn challenge was performed on Day −1 and Day 1 at 1 hour postdose and induced a plasma aldosterone peak at the 0 to 3 hr time interval.

On both Day −1 and Day 1 at 6 hours postdose subjects were asked to stand for 30 minutes, which induced a plasma aldosterone peak at the 4 to 12 hr time interval.

The decreases in plasma aldosterone levels were associated with dose-dependent decreases in both aldosterone and tetrahydro-aldosterone urine excretion, which started on Day 1 and remained constant on Day 2. A close to maximum effect of change in aldosterone urine excretion was also achieved at the 10 mg dose.

The precursors of aldosterone remained unchanged until doses ≥90 mg, at which point 11-DOC (the precursor to both aldosterone and cortisol) began to increase while corticosterone levels remained unchanged, suggesting partial inhibition of this pathway at doses well above the anticipated therapeutic range.

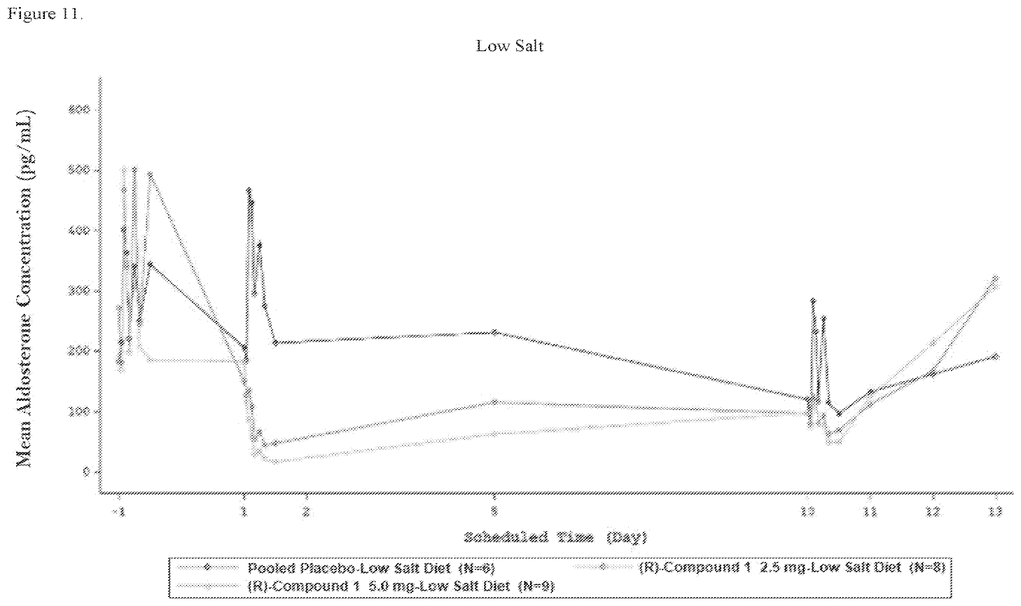

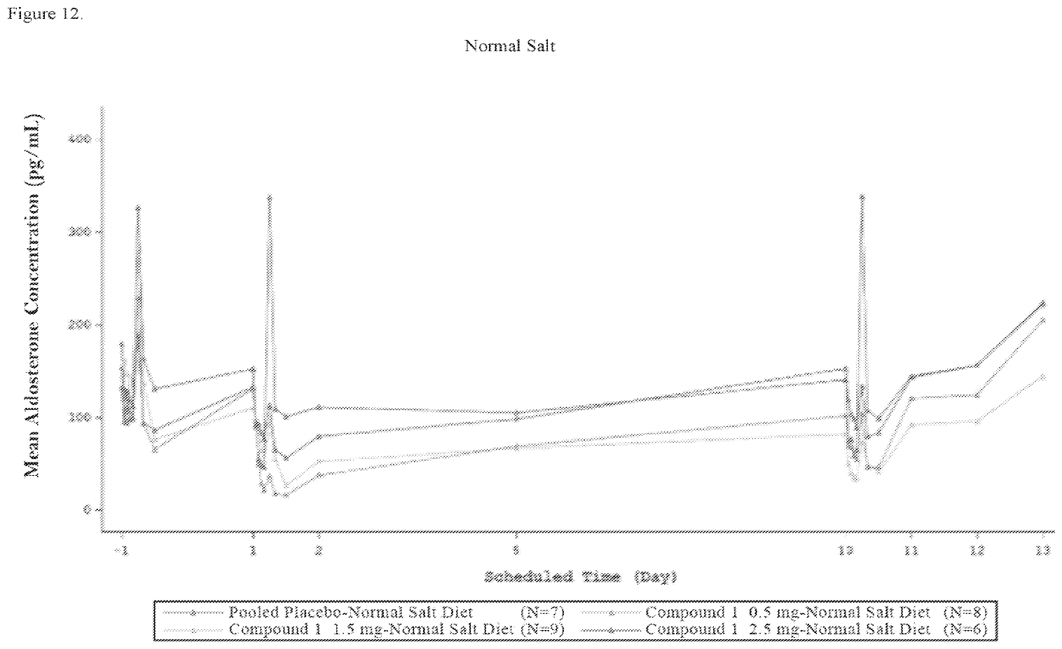

FIG. 11 and FIG. 12 display the plots of mean 11-deoxycorticosterone concentrations over time by treatment for the normal salt diet and low salt diet treatment groups, respectively, for the PD Population. Treatment with (R)-Compound 1 resulted in increases in 11-deoxycorticosterone on Day 10 as compared to Day −1 in both normal salt diet and low salt diet treatment groups and in the presence and absence of Cortrosyn stimulation.

The dietary sodium and potassium limits during the run-in period for Cohort 1 were 50 to 60 mEq Na⁺/day and 70 to 100 mEq K⁺/day, respectively. On Day 1 of the treatment period, the dietary restrictions were changed to 65 to 70 mEq Na⁺/day and 70 to 100 mEq K⁺/day for Cohort 1 and remained as such until the end of the treatment period. These limits of 65 to 70 mEq Na⁺/day and 70 to 100 mEq K+/day were applied to Cohort 2 from the start ofthe run-in period through completion ofthe treatment period. In both cohorts, additional minor modifications to salt intake were made on an individual basis, as needed, to manage electrolyte levels.

### Multiple Dose

Following administration of multiple doses of (R)-compound 1≤5 mg, there were no apparent differences in cortisol or 11-deoxycortisol levels as compared to placebo on either Day 1 or Day 10. These findings are consistent in the presence of Cortrosyn challenge (which occurred with the low salt treatment groups) and in the absence of the challenge (normal salt groups) (Table 13).

TABLE 13
Analysis of Plasma Cortisol and 11-Deoxycortisol
AUCO-12 Estimated Percent Change from Baseline

| | | | | Day 1 | | Day 10 | |
|---|---|---|---|---|---|---|---|
| | Parameter/Treatment | | | LS | | LS | |
| | Group | | | Mean | 90% CI | Mean | 90% CI |
| Cortisol | (R)- | Normal | 0.5 | −4.43 | (−17.45, 10.65) | −19.53 | (−32.41, −4.20) |
| | Compound | salt diet | mg | | | | |
| | 1 | | 1.5 | 0.62 | (−12.02, 15.06) | −4.45 | (−18.35, 11.82) |
| | | | mg | | | | |
| | | | 2.5 | −0.52 | (−15.60, 17.24) | −9.23 | (−26.53, 12.13) |
| | | | mg | | | | |
| | | Low | 2.5 | −3.91 | (−12.16, 5.11) | −38.02 | (−47.51, −26.81) |
| | | salt diet | mg | | | | |
| | | | 5.0 | 4.04 | (−4.64, 13.50) | −37.25 | (−46.59, −26.27) |
| | | | mg | | | | |
| | Pooled | Normal salt | | 2.66 | (−12.25, 20.11) | 2.10 | (−16.70, 25.15) |
| | Placebo | diet | | | | | |
| | | Low salt diet | | 12.79 | (1.55, 25.26) | −38.72 | (−49.54, −25.58) |
| 11- | (R)- | Normal | 0.5 | −0.57 | (−11.10, 11.20) | −6.77 | (−20.52, 9.35) |
| Deoxycortisol | Compound | salt diet | mg | | | | |
| | 1 | | 1.5 | 2.39 | (−7.58, 13.44) | 1.82 | (−12.00, 17.81) |
| | | | mg | | | | |
| | | | 2.5 | 12.73 | (−0.62, 27.88) | −2.72 | (−20.03, 18.34) |
| | | | mg | | | | |
| | | Low | 2.5 | 21.63 | (12.44, 31.56) | −25.69 | (−33.76, −16.63) |
| | | salt diet | mg | | | | |
| | | | 5.0 | 36.67 | (26.85, 47.24) | 18.84 | (6.56, 32.54) |
| | | | mg | | | | |
| | Pooled | Normal salt | | 3.15 | (−8.71, 16.54) | 6.18 | (−11.93, 28.02) |
| | | diet | | | | | |
| | Placebo | Low salt diet | | 33.04 | (21.43, 45.76) | −40.69 | (−48.11, −32.21) |

Measurements following a subject's early termination from study drug were excluded. LS means and 90% CIs were derived using an ANOVA model on change in AUC from baseline (Day −1) to Days 1 and 10. AUC values were logarithm-transformed prior to analysis and the model estimates were exponentiated to present model estimates in the original scale.
ANOVA = analysis of variance; AUC₀₋₁₂ = area under the pharmacodynamic effect-time curve from time 0 to 12 hours postdose; CI = confidence interval; LS = least square.

Consistent with observations from the AUC data for cortisol, (R)-compound 1 had no apparent effect on response to the Cortrosyn challenge, with Day 1 and Day 10 responses in (R)-compound 1-treated subjects being similar to their response at baseline and to the response in subjects receiving placebo.

### Safety of (R)-Compound 1 in Humans

AEs were reported for few subjects and the incidence was not dose dependent (Table 14). No severe AEs, SAEs, withdrawals due to AEs, or deaths were noted. Overall, the most frequently reported AEs across dose levels were headache, nasopharyngitis, diarrhea, and nausea; however, the only events reported by >1 subject at any dose level were toothache (2 subjects [12.5%] with placebo), nasopharyngitis (2 subjects [12.5%] with placebo), and headache (2 subjects [33.3%] with 180 mg). The majority of AEs were considered not related to study drug. Two events of moderate gastroenteritis were reported (with 180 mg and placebo) and all other AEs were mild in intensity.

TABLE 14
Overview of Adverse Events: Part 1

| | Number (%) of Subjects | | | | | | | |
|---|---|---|---|---|---|---|---|---|
| Type of | 1 mg | 3 mg | 10 mg | 30 mg | 90 mg | 180 mg | 360 mg | Placebo |
| AE | N = 9 | N = 9 | N = 6 | N = 6 | N = 6 | N = 6 | N = 6 | N = 16 |
| Any AE | 1 (11.1) | 3 (33.3) | 2 (33.3) | 2 (33.3) | 2 (33.3) | 4 (66.7) | 2 (33.3) | 9 (56.3) |
| Related AE | 0 | 0 | 2 (33.3) | 0 | 1 (16.7) | 2 (33.3) | 1 (16.7) | 1 (6.3) |

AE = adverse event; N = number of subjects.

Isolated markedly abnormal safety laboratory values were reported but no dose-dependent pattern was apparent. No AEs related to markedly abnormal safety laboratory values were reported. Mean decreases in hemoglobin, hematocrit, red blood cell count, urine osmolality, and urine-specific gravity were observed at all dose levels including placebo, but with no clear dose-dependency.

No clinically significant or dose-dependent changes were observed over time for the ECG parameters, vital signs, or ocular assessments. A transient BW decrease was observed for the active treatment groups compared to placebo but without dose dependency.

There were no dose-related increases in the incidence of AEs (Table 15). No severe AEs, SAEs, withdrawals due to AEs, or deaths were reported. A larger proportion of subjects reported AEs during the 3 mg IV dose (75%) compared to any of the oral doses (maximum of 66.7% with 3 mg during the low salt diet and with 10 mg during both the low and normal salt diet). Overall, the most frequently reported AEs were nasopharyngitis, asthenia, and dizziness. AEs reported by >1 subject at any dose level and with either salt diet were asthenia (2 subjects [33.3%] with 10 mg low salt diet, 2 subjects [33.3%] with placebo low salt diet), nasopharyngitis (3 subjects [37.5%] with 3 mg IV, 2 subjects [33.3%] with 10 mg low salt diet), dizziness (2 subjects [33.3%] with 3 mg low salt diet) and gingival pain (2 subjects [33.3%] with 10 mg low salt diet). Only 1 AE (dizziness) reported by a subject receiving 10 mg (R)-compound 1 under normal salt diet conditions was considered related to the study drug. One case of fractured coccyx and one case of concussion (both reported for subjects who received the IV dose) were moderate in intensity and all other AEs were mild in intensity.

TABLE 15
Overview of Adverse Events: Part 2

| | Number (%) of Subjects | | | | | | | | |
|---|---|---|---|---|---|---|---|---|---|
| | 1 mg | 1 mg | 3 mg | 3 mg | 3 mg | 10 mg | 10 mg | Placebo | Placebo |
| Type of | (low) | (normal) | (low) | (normal) | IV | (low) | (normal) | (low) | (normal) |
| AE | N = 6 | N = 6 | N = 6 | N = 6 | N = 8 | N = 6 | N = 6 | N = 6 | N = 6 |
| | 2 | 3 | 4 | 2 | 6 | 4 | 4 | 2 | 3 |
| Any AE | (33.3) | (50.0) | (66.7) | (33.3) | (75.0) | (66.7) | (66.7) | (33.3) | (50.0) |
| Related | 0 | 0 | 0 | 0 | 0 | 0 | 1 | 0 | 0 |
| AE | | | | | | | (16.7) | | |

AE = adverse event; low = low salt diet; N = number of subjects; normal = normal salt diet.
Source: WP28586 Clinical Study Report

No clinically significant or dose-dependent changes were observed over time in the safety laboratory test results. Isolated markedly abnormal safety laboratory values were reported but no dose-dependent pattern was apparent. Mean increases in hemoglobin, hematocrit, and red blood cell count were observed at all dose levels except placebo during the low salt diet. At each dose level, the increase was greater during the normal salt diet than during the low salt diet conditions. The 3 mg IV dose had smaller increases compared to the 3 mg oral dose (both under low and normal salt diet conditions). A decrease in mean urine osmolality and urine specific gravity was noted at all dose levels, except 1 and 3 mg under low salt diet conditions.

There were no clinically relevant trends observed for the change in ECG parameters or vital signs over time or between dose levels. Mean BW decreased from baseline in all dose levels including placebo from Day 1 until Day 4 and returned to normal at the follow-up visit. The decreases were not dose-dependent; however, larger decreases were noted during the low salt diet compared to the normal salt diet for the active treatment groups.

(R)-compound 1 administered QD for 10 days was well tolerated by healthy subjects under low salt (2.5 mg and 5 mg of (R)-compound 1) and normal salt conditions (0.5, 1.5, and 2.5 mg of (R)-compound 1). There were no deaths, SAEs, or TEAEs leading to withdrawal. The most common TEAEs following administration of multiple doses of (R)-compound 1 were headache (4 subjects), postural dizziness (3 subjects), and dizziness (2 subjects). The TEAEs in the placebo group included nausea (1 subject), non-sustained ventricular tachycardia (1 subject), and palpitations (1 subject). All AEs following administration of (R)-compound 1 were mild in nature.

The overall incidence of AEs in subjects receiving (R)-compound 1 was comparable to that in subjects receiving placebo and there was no consistent trend towards an increase in incidence or relatedness of AE with increase in dose (Table 16).

TABLE 16
Overview of Adverse Events Following Multiple Oral Doses of (R)-compound 1

| | Number (%) of Subjects | | | | | | | | |
|---|---|---|---|---|---|---|---|---|---|
| | 0.5 mg | 1.5 mg | 2.5 mg | 2.5 mg | 5 mg | Total (R)- | Placebo | Placebo | Total |
| | NS | NS | NS | LS | LS | Compound 1 | NS | LS | Placebo |
| | N = 9 | N = 9 | N = 6 | N = 9 | N = 9 | N = 42 | N = 8 | N = 6 | 14 |
| Any | 1 | 1 | 3 | 3 | 3 | 11 | 1 | 2 | 3 |
| TEAE | (11.1) | (11.1) | (50) | (33.3) | (33.3) | (26.2) | (12.5) | (33.3) | (21.4) |
| Related | 1 | 0 | 2 | 2 | 1 | 6 | 1 | 2 | 3 |
| TEAE | (11.1) | (0) | (33.3) | (22.2) | (11.1) | (14.3) | (12.5) | (33.3) | (21.4) |

TEAE = treatment-emergent adverse events; LS = low salt; NS = normal salt; N = number of subjects.

No clinically significant changes were observed over time in the safety laboratory test results. Isolated markedly abnormal safety laboratory values were reported, which were likely due to a combination of the protocol-specified dietary sodium requirements and the effects of (R)-compound 1, but no dose-dependent pattern was apparent. Mild, dose-dependent decreases in plasma sodium levels and increases in plasma potassium levels were observed with corresponding changes in urine sodium and potassium levels. Of note, despite an initial increase in the urine sodium:potassium ratio, indicating that the sodium loss in urine is greater than the potassium retention, the ratio normalized by Day 10, suggesting that the balance between sodium excretion and potassium absorption was restored. The change in the ratio appears to be mediated by a greater elimination of sodium in the urine on day 1 as compared to sodium elimination in the urine on Day 10 as potassium appears not to change over the course of the 10-day treatment period.

Consistent with observations for other RAAS-modifying agents, increases in blood urea nitrogen and creatinine were also observed following administration of (R)-compound 1 along with a mild reduction (<15%) in glomerular filtration rate. The presence of an increased blood urea nitrogen:creatinine ratio with reduced glomerular filtration rate suggests that (R)-compound 1 is producing a mild diuretic effect. Finally, subjects receiving (R)-compound 1 experienced more pronounced decreases in body weight and body mass index (BMI) as compared to subjects receiving placebo, in all likelihood due to the previously noted mild diuretic effects. Values returned to normal by the time of the follow-up visit.

There were no clinically relevant ECG findings, including no meaningful changes in QTcF. There were also no clinically relevant changes in vital signs. However, there were some slight trends towards mild, drug-induced decreases in seated and orthostatic BP and moderate increases in orthostatic heart rate. These trends were not consistently dose-dependent though the most pronounced effects on heart rate were observed at the 5 mg dose level.

Mean BW and body mass index decreased slightly (≤2 kg or <0.65 kg/m²) from baseline in all dose levels including placebo during the treatment period then returned to normal at the follow-up visit. There was no clear dose-dependency in the observed decreases however the decrease in subjects receiving (R)-compound 1 was greater than that in subjects receiving placebo.

### Example 7

The placebo-controlled Phase 2 study is to evaluate the efficacy and safety of multiple dose strengths of (R)-Compound 1 in the treatment of patients with rHTN on a stable background hypertensive regimen. Efficacy will be analyzed by the change from baseline of SBP, DBP, PK, and PD parameters. AEs will be monitored from the time of informed consent until the end of the Follow-up Period. All patients will receive their background antihypertensive medications, unless requested otherwise through a Central Pharmacy from the time of the SB-RI Period (Visit 3) through the End of Treatment Visit (Visit 11).

### Study Purposes

### Part A

Purpose: To demonstrate that at least one dose strength of (R)-Compound 1 is superior to placebo in mean change from baseline in seated SBP after 12 weeks of treatment in patients with rHTN.

### Other Purposes

To evaluate the change from baseline in mean seated DBP with each of the selected dose strengths of (R)-Compound 1 compared to placebo after 12 weeks of treatment in patients with rHTN; and
To evaluate the percentage of patients achieving a seated BP response <130/80 mmHg with each of the selected dose strengths of (R)-Compound 1 compared to placebo after 12 weeks of treatment for rHTN.
To evaluate vital signs, standing BP and heart rate, physical examinations, electrocardiography, weight measurement, and clinical laboratory evaluations, including standard safety chemistry panel, hematology, coagulation, and urinalysis;
To evaluate TEAEs;
To evaluate TEAEs leading to premature discontinuation of study drug;
To evaluate treatment-emergent marked laboratory abnormalities; and
To evaluate the change in standing SBP and DBP (measured pre-dose at the clinical site) from baseline to End of Treatment (Visit 11).

The PK-PD objective is to evaluate exposure-response relationships (pharmacokinetic-pharmacodynamics) of (R)-Compound 1 using measures of safety, PD, and/or efficacy.

### Part B

Part B is a sub-study to characterize the PK of (R)-Compound 1 in patients with rHTN and to obtain additional data to support the PK-PD objective of Part A.

### Study Description

### (i) Summary of Study Design

This is a Phase 2, 2-part, randomized, double-blind, placebo-controlled, multicenter, parallel-group, dose-ranging study to evaluate the efficacy and safety of multiple dose strengths of (R)-Compound 1 as compared to placebo after 12 weeks of treatment in patients with rHTN.

Patients with rHTN will be defined as being on a stable regimen of ≥3 antihypertensive agents, 1 of which is a diuretic, with a mean seated BP≥130/80 mmHg.

The safety of (R)-Compound 1 will be assessed from the time of informed consent until the end of the Follow-up Period. Patients will be followed for efficacy and adherence throughout the Double-Blind Treatment Period. PD variables analyzed during the study may include, but are not limited to, measures of aldosterone and its precursors, cortisol and its precursor, PRA, and calculation of ARR and UACR. PK variables analyzed during the study will include plasma concentrations of (R)-Compound 1 and any measured metabolites.

Patients should not exercise, smoke, or consume caffeinated beverages or food for at least 2 hours prior to each clinical site visit. All clinical site visits should occur between 6:00 a.m. and 11:00 a.m.

### Part A

Part A is a Phase 2, randomized, double-blind, placebo-controlled, multicenter, parallel-group, dose-ranging study to evaluate the efficacy and safety of multiple dose strengths of (R)-Compound 1 as compared to placebo after 12 weeks of treatment in patients with rHTN.

### (i) Adaptive Design

During the Double-Blind Treatment Period, eligible patients will be randomized 1:1:1 into 1 of the 3 treatment groups (2 active [1 mg and 2 mg (R)-Compound 1] and 1 placebo). After approximately the first 25 randomized patients per group reach approximately 4 weeks of study drug dosing, emerging data will be evaluated and reported on cumulative SAEs. Based on assessments, the next dose level) to be studied is 0.05 mg QD. Patient enrollment in the study will not stop during the first review.

Part A will enroll patients using a randomization plan to allow for approximately equal distribution between the treatment groups at the conclusion of the study.

### (ii) Study Visits

Part A of the study will consist of 4 periods:
A Screening Period (Screening Visit [Visit 1] and Telephone Call 1 [Visit 2]) of up to 8 weeks;
An SB-RI Period (Visit 3) of 2 weeks;
A Double-Blind Treatment Period (Visits 4 to 11) of 12 weeks; and
A Follow-up Period (Telephone Call 2 [Visit 12]) of up to 1 week.

Patients will complete at least 12 total visits over a period of approximately 6 months, including 10 clinic visits and 2 Telephone Visits.

(iii) Screening Period (Visits 1 and 2)

Patients will provide informed consent at the Screening Visit (Visit 1) and undergo assessment for Inclusion/Exclusion Criteria.

Patients taking an MRA or a potassium sparing diuretic (e.g., triamterene, amiloride, etc.) as an antihypertensive agent must be willing to discontinue this agent for study eligibility. Potassium sparing diuretic must be discontinued and replaced with a non-potassium sparing diuretic. If an MRA is a fourth antihypertensive agent, a replacement medication does not need to be initiated. If an MRA is a third antihypertensive agent, a replacement medication must be initiated. All patients who remain on a stable regimen of ≥3 antihypertensive agents, including a non-potassium sparing diuretic for at least two weeks, will be eligible to enter the SB-RI Period. Eligible patients will be contacted via Telephone Call 1 (Visit 2) to be informed about study qualification and to schedule their SB-RI Period.

Patients may have a mean seated BP<130/80 mmHg at Screening if taking an MRA as part of their antihypertensive regimen; however, the mean seated BP must be ≥130/80 mmHg at SB-RI Period (Visit 3) after MRA discontinuation, with or without replacement medication, for study eligibility.

Screening laboratory evaluations, if abnormal, may be repeated once for eligibility purposes before excluding the patient. A patient who is screened and does not meet the study Inclusion/Exclusion Criteria or Randomization Criteria (screening failure) may be rescreened no less than 5 days after the last study visit.

### (iv) SB-RI Period (Visit 3)

The SB-RI Period will last approximately 2 weeks (±2 days). The objective of this period is to determine whether medication adherence is a factor in patients not achieving goal BP.

All patients will receive their background antihypertensive medications, unless requested otherwise through a Central Pharmacy from Visit 3 through Visit 11. Clinical sites will send prescriptions for background antihypertensive medications to the Central Pharmacy at Visit 2 or at least 1 week before Visit 3. These medications will be delivered directly to the clinical site. Background antihypertensive medications and study drug (single-blind placebo) will be dispensed at Visit 3.

### (v) Double-Blind Treatment Period (Visits 4 to 11)

Measurements of efficacy and safety variables at Randomization (Visit 4) will constitute “baseline” measurements. Measurements of efficacy and safety variables recorded prior to study drug administration at the clinical site will constitute “pre-dose” measurements.

Patients with ≥70% and ≤120% adherence (based on pill counts) to each antihypertensive medication and study drug during the SB-RI Period, and a baseline mean seated BP of ≥130/80 mmHg will continue with Randomization eligibility procedures.

Eligible patients will be randomized 1:1:1 into 1 of the 3 treatment groups (2 active [1 mg and 2 mg (R)-Compound 1] and 1 placebo). After approximately the first 25 randomized patients per group reach approximately 4 weeks of study drug dosing, emerging data is evaluated and reported on cumulative SAEs. Based on assessments, the next dose level to be studied will be 0.05 mg QD. Following review, Part A will enroll patients using a randomization plan to allow for approximately equal distribution between the treatment groups at the conclusion of the study.

Study drug ((R)-Compound 1 or placebo) dispensing may occur at any time starting at Visit 4 and before Visit 11. Clinical sites will send prescriptions for background antihypertensive medications to the Central Pharmacy at Visit 4 and these medications will be dispensed at Visit 5 or Visit 6. It is expected that the patient's background antihypertensive regimen remains unchanged, and is not titrated, during the treatment period. On clinical site visit days, patients will self-administer the morning dose of background hypertensive medications at home and withhold the study drug. At the clinical site, patients will self-administer the morning dose of study drug to be witnessed by site staff after completion of pre-dose evaluations and laboratory sampling. Between clinical site visits, patients will continue taking their study drug QD by mouth at approximately the same time each morning. The primary endpoint evaluation will take place at the End of Treatment (Visit 11).

Pre-dose blood samples for PD analysis will be collected at Visits 4, 7, 8, and 11. Pre-dose blood samples for PK analysis will be collected at Visits 8 and 11. Safety and adherence will be monitored all throughout the Double-Blind Treatment Period.

Urine for PD and electrolyte measurements will be collected starting 24 hours prior to dosing at Visit 4 as well as 24 hours prior to dosing at Visit 11/EOT.

### (vi) Follow-up Period (Telephone Call 2, Visit 12)

Patients will have a Telephone Call 2 (Visit 12) at 1 week±3 days following the last dose of the study drug to assess adverse events (AEs) and concomitant medications including background antihypertensive regimen since study completion.

Study visits will follow the Schedule of Procedures. See, Tables 17A and 17B.

TABLE 17A
SCHEDULE OF PROCEDURES

| | Screening Period | | |
|---|---|---|---|
| | Screening | Telephone | SB-RI |
| | Visit | Call¹ | Period |
| Visit^{a} | 1 | 2 | 3 |
| Week | −10 to −2 | −4 to −2 | −2 to 1 |
| Day | −70 to −14 | −28 to −14 | −14 to 1 |
| (±Visit Window) | (±2) | (±2) | (±2) |
| Informed consent^{b} | X | | |
| Inclusion/Exclusion^{c} | X | | X |
| Demographics | X | | |
| Medical/surgical history | X | | |
| Adverse events | X | X | X |
| Prior/concomitant medications | X^{g} | X | X |
| Weight, height, and BMI^{h} | X | | X |
| Vital signsⁱ | X | | X |
| Seated BPⁱ | X^{j} | | X |
| Standing BP and heart rate^{l} | X | | X |
| Complete physical examination^{m} | X | | |
| Limited physical examinationⁿ | | | X |
| 12-lead ECG^{o} | X | | |
| Urinalysis | X | | X |
| Standard safety chemistry panel, | X | | X |
| hematology, coagulation | | | |
| HbA1c | X | | |
| HIV, HBsAg, HCV screen | X | | |
| Pregnancy test^{p} | X | | |
| FSH^{q} | X | | |
| Dispense study drug | | | X^{w} |
| Dispense antihypertensive | | | X |
| medications | | | |
| Administer study drug^{y} | | | X |
| Adherence counselling | | X | X |
| Provide instructions for next visit^{dd} | | X | X |
| Provide materials for next 24- hour | | | X |
| urine collection^{gg} | | | |

^{a}Unscheduled Visits may be scheduled at any time during the study period.
^{b}Written informed consent must be obtained before any protocol-specific procedures are performed.
^{c}Screening laboratory evaluations, if abnormal, may be repeated once for eligibility purposes before excluding the patient. A patient who is screened and does not meet the study Inclusion/Exclusion Criteria or Randomization Criteria (screening failure) may be rescreened no less than 5 days after the last study visit.
^{g}Patients taking an MRA or a potassium sparing diuretic (e.g., triamterene, amiloride, etc.) as an antihypertensive agent must be willing to discontinue this agent for study eligibility. The potassium sparing diuretic must be discontinued and replaced with a non-potassium sparing diuretic. If an MRA is a fourth antihypertensive agent, a replacement medication does not need to be initiated. If an MRA is a third antihypertensive agent, a replacement medication must be initiated. All patients who remain on a stable regimen of ≥ 3 antihypertensive agents, including a non-potassium sparing diuretic, for at least two weeks, will be eligible to enter the SB-RI Period.
^{h}Height will be collected at Screening only and will be used to calculate BMI at subsequent visits.
ⁱPatient should be seated for at least 5 minutes in the examination room before measurement of vital signs and BP.
^{j}BP will be measured in both upper arms (3 times/arm) using an appropriately sized cuff to detect possible laterality differences. The arm with the higher mean value will then be used to take the Screening BP measurements (at least 5 minutes after determining laterality) and for all subsequent measurements.
^{l}Once the seated BP has been determined, the patient will be asked to stand and after 60 seconds a single standing BP and heart rate measurement will be obtained.
^{m}A complete physical examination will consist of general appearance, skin, head, eyes, ears, mouth, oropharynx, neck, heart, lungs, abdomen, extremities, and neuromuscular system.
ⁿA limited physical examination will consist of a minimum of general appearance, skin, heart, lungs, and abdomen.
^{o}Perform 12-lead ECG after the patient has been resting in the supine position for at least 10 minutes and after measuring vital signs and BP.
^{p}For female patients of childbearing potential (ovulating, pre-menopausal, and not surgically sterile), serum pregnancy tests will be performed at Screening, EOT, and ET Visits. A POC pregnancy test will be performed at Randomization (Visit 4) to assess eligibility.
^{q}FSH levels will be measured only for female patients who are post-menopausal for at least 1 year at Screening and are not surgically sterile.
^{w}Study drug (a single-blind placebo) will be dispensed to cover the SB-RI period and dosing of the study drug will have been completed 1 day prior to Visit 4 for most patients.
^{y}During clinical site visits, patients will self-administer the study drug in the clinic to be witnessed by site staff after completion of pre-dose evaluations and laboratory sampling. Starting the following morning, patients will self-administer the study drug by mouth QD at home at approximately the same time each morning.
^{z}Site staff will calculate treatment adherence based on pill counts. Between clinical site visits, site staff will utilize the electronic diary to ensure patient's adherence to background antihypertensive regimen and study drug.
^{dd}Instruct patients to take their scheduled morning doses of background antihypertensive medications at home and to hold their dose of study drug on the morning of their next visit. Patients must bring their study drug and background antihypertensive medications to the clinical site at all visits. Patients should not exercise, smoke, or consume caffeinated beverages or food for at least 2 hours prior to the next visit.
^{gg}Patients will be instructed to begin collecting all urine starting 24 hours prior to Visit 4 and 11 and to bring the entire sample to the clinical site.

TABLE 17B
SCHEDULE OF PROCEDURES

| | | | | | | | | | Follow- |
|---|---|---|---|---|---|---|---|---|---|
| | | | | | | | | | up Period |
| | | | | | | | | | Telephone |
| | Double-Blind Treatment Period | | | | | | | | Call 2 |
| | Visit^{a} | | | | | | | | |
| | | | | | | | | EOT/ | |
| | | | | | | | | ET 11/ | |
| | 4 | 5 | 6 | 7 | 8 | 9 | 10 | NA | 12 |
| | Week | | | | | | | | |
| | 1 | 1 | 2 | 3 | 4 | 7 | 10 | 13/NA | |
| Day | 1 | 3 | 8 | 15 | 22 | 43 | 64 | 85 (±2)/ | 14 |
| (±Visit Window) | (±2) | (±2) | (±2) | (±2) | (±2) | (±2) | (±2) | NA | 92 (±3) |
| Inclusion/Exclusion^{c} | X^{d} | | | | | | | | |
| Adverse events | X | X | X | X | X | X | X | X | X |
| Prior/concomitant | X | X | X | X | X | X | X | X | X |
| medications | | | | | | | | | |
| Weight, height, and | X | X | X | X | | X | X | X | |
| BMI^{h} | | | | | | | | | |
| Vital signsⁱ | X | X | X | X | | X | X | X | |
| Seated BPⁱ | X^{k} | X | X | X | X | X | X | X | |
| Standing BP and heart | X | X | X | X | X | X | X | X | |
| rate^{l} | | | | | | | | | |
| Complete physical | | | | | | | | X | |
| examination^{m} | | | | | | | | | |
| Limited physical | X | X | X | X | X | X | X | | |
| examinationⁿ | | | | | | | | | |
| 12-lead ECG^{o} | X | | | | | | | X | |
| Urinalysis | X | X | X | X | X | X | X | X | |
| Standard safety | X | X | X | X | X | X | X | X | |
| chemistry panel, | | | | | | | | | |
| hematology, | | | | | | | | | |
| coagulation | | | | | | | | | |
| Pregnancy test^{p} | X | | | | | | | X | |
| FSH^{q} | | | | | | | | | |
| PD blood sampling^{r} | X | | | X | X | | | X | |
| PK blood sampling^{s} | | | | | X | | | X^{t} | |
| Dispense study drug | ← X^{x} → | | | | | | | | |
| Dispense | | X^{v} | X^{v} | | | | | | |
| antihypertensive | | | | | | | | | |
| medications | | | | | | | | | |
| Randomization | X | | | | | | | | |
| Administer study | X | X | X | X | X | X | X | X | |
| drug^{y} | | | | | | | | | |
| Assess treatment | X | X | X | X | X | X | X | X | |
| adherence^{z} | | | | | | | | | |
| Adherence counselling | X | X | X | X | X | X | X | | |
| Collect unused study | X | | | | | | | X | |
| drug | | | | | | | | | |
| Provide instructions | X | X | X | X | X | X | X^{ee} | | |
| for next visit^{dd} | | | | | | | | | |
| PGx sample^{ff} | ← X → | | | | | | | | |
| Provide materials for | | | | | | | X | | |
| next 24- hour Urine | | | | | | | | | |
| Collection^{gg} | | | | | | | | | |
| Obtain Sample from | X | | | | | | | X | |
| 24-hour Urine | | | | | | | | | |
| Collection^{hh} | | | | | | | | | |

^{a}Unscheduled Visits may be scheduled at any scheduled at any time during the study period.
^{c}Screening laboratory evaluations, if abnormal, may be repeated once for eligibility purposes before excluding the patient. A patient who is screened and does not meet the study Inclusion/Exclusion Criteria or Randomization Criteria (screening failure) may be rescreened no less than 5 days after the last study visit.
^{d}Patients must meet the Randomization Criteria in addition to the Inclusion/Exclusion Criteria.
^{h}Height will be collected at Screening only and will be used to calculate BMI at subsequent visits.
ⁱPatient should be seated for at least 5 minutes in the examination room before measurement of vital signs and BP.
^{k}If the lowest and highest SBP measurements are >15 mmHg apart, additional readings should be performed. The last 3 consecutive, consistent SBP measurements will be averaged to determine the final value to be used to assess Randomization eligibility. If the lowest and highest SBP measurements are >20 mmHg apart after a total of 6 measurements, the measurements will not be used to assess study eligibility, but measurements may be reassessed after at least 72 hours. If the lowest and highest SBP values remain >20 mmHg apart after 6 measurements at a subsequent assessment, the patient will be excluded from the study.
^{l}Once the seated BP has been determined, the patient will be asked to stand and after 60 seconds a single standing BP and heart rate measurement will be obtained.
^{m}A complete physical examination will consist of general appearance, skin, head, eyes, ears, mouth, oropharynx, neck, heart, lungs, abdomen, extremities, and neuromuscular system.
ⁿA limited physical examination will consist of a minimum of general appearance, skin, heart, lungs, and abdomen.
^{o}Perform 12-lead ECG after the patient has been resting in the supine position for at least 10 minutes and after measuring vital signs and BP.
^{p}For female patients of childbearing potential (ovulating, pre-menopausal, and not surgically sterile), serum pregnancy tests will be performed at Screening, EOT, and ET Visits. A POC pregnancy test will be performed at Randomization (Visit 4) to assess eligibility.
^{q}FSH levels will be measured only for female patients who are post-menopausal for at least 1 year at Screening and are not surgically sterile.
^{r}Pre-dose blood samples for PD analysis will be collected at specified visits.
^{s}Pre-dose blood samples for PK analysis will be collected within approximately 15 minutes prior to dosing.
^{t}Patients who provide written informed consent to participate in the optional Part B sub-study will undergo post-dose PK blood sampling at the following timepoints at Visit 11: 1, 2, 3, 4, 6, and 8 hours. A ±5 minutes window is permitted for the collection of post-dose PK samples.
^{v}Clinical sites will send prescriptions for background antihypertensive medications to the Central Pharmacy on the day of randomization (Visit 4) to dispense at Visit 5 or Visit 6. The supply of background antihypertensive medications provided to the patient at Visit 5 or Visit 6 should be adequate to cover until Visit 11 (EOT).
^{x}Randomized study drug ((R)-Compound 1 or placebo) dispensation may occur at any time starting at Visit 4 and before Visit 11 (EOT).
^{dd}Instruct patients to take their scheduled morning doses of background antihypertensive medications at home and to hold their dose of study drug on the morning of their next visit. Patients must bring their study drug and background antihypertensive medications to the clinical site at all visits. Patients should not exercise, smoke, or consume caffeinated beverages or food for at least 2 hours prior to the next visit.
^{y}During clinical site visits, patients will self-administer the study drug in the clinic to be witnessed by site staff after completion of pre-dose evaluations and laboratory sampling. Starting the following morning, patients will self-administer the study drug by mouth QD at home at approximately the same time each morning.
^{z}Site staff will calculate treatment adherence based on pill counts. Between clinical site visits, site staff will utilize the electronic diary to ensure patient's adherence to background antihypertensive regimen and study drug.
^{dd}Instruct patients to take their scheduled morning doses of background antihypertensive medications at home and to hold their dose of study drug on the morning of their next visit. Patients must bring their study drug and background antihypertensive medications to the clinical site at all visits. Patients should not exercise, smoke, or consume caffeinated beverages or food for at least 2 hours prior to the next visit.
^{ee}Patients participating in the optional Part B sub-study should be instructed to present to the clinical site at Visit 11 in a fasting state for 8 hours relative to study drug administration and will remain so for 4 hours after study drug administration. Patients will not be able to eat or drink other than water during the 12 hours of fasting.
^{ff}For patients who provide written informed consent to participate in the optional pharmacogenomic assessment, a blood sample will be collected at any time after Randomization.
^{gg}Patients will be instructed to begin collecting all urine starting 24 hours prior to Visit 4 and 11 and to bring the entire sample to the clinical site.
^{hh}A 24-hour urine collection can be repeated if it is suspected that the sampling is insufficient and the patient is within the visit window.
NA = not applicable

### Part B

After taking part in the prior visits and procedures of Part A, approximately 10-15% of the patients are expected to participate in the optional Part B sub-study at the End of Treatment (Visit 11).

Patients participating in Part B will present to the clinical site at Visit 11 in a fasted state for 8 hours relative to study drug administration and will remain so for 4 hours after study drug administration. Patients will not be able to eat or drink other than water during the 12 hours of fasting. Additional post-dose PK sampling will be performed at the following timepoints at Visit 11: 1, 2, 3, 4, 6, and 8 hours. A ±5 minutes window is permitted for the collection of post-dose PK samples.

### Selection and Withdrawal of Patients

All Inclusion, Exclusion, and Randomization Criteria for patients participating in Part A are applicable to patients participating in Part B. No new patients will be enrolled into Part B, and there are no additional criteria for participation in Part B.

### (i) Inclusion Criteria

Patients who meet all of the following criteria will be eligible to participate:
1. Are adult male and female patients ≥18 years;
2. Is on a stable regimen of ≥3 antihypertensive agents at the time of Screening, 1 of which is a diuretic, at MTD based on Investigator judgment—patients taking an MRA or a potassium sparing diuretic (e.g., triamterene, amiloride, etc.) as an antihypertensive agent must be willing to discontinue this agent for study eligibility. The potassium sparing diuretic must be discontinued and replaced with a non-potassium sparing diuretic. If an MRA is a fourth antihypertensive agent, a replacement medication does not need to be initiated. If an MRA is a third antihypertensive agent, a replacement medication must be initiated. All patients who remain on a stable regimen of ≥3 antihypertensive agents, including a non-potassium sparing diuretic, for at least two weeks, will be eligible to enter the SB-RI Period;
Anti-anginal nitrates including nitroglycerine, isosorbide mononitrate, and isosorbide dinitrate are not considered antihypertensive agents;
3. Has a mean seated BP 130/80 mmHg—Mean seated BP is defined as the average of 3 seated BP measurements at any single clinical site visit. Patients may have mean seated BP<130/80 mmHg at Screening if taking an MRA as part of their antihypertensive regimen; however, the mean seated BP must be ≥130/80 mmHg at Visit 3 after discontinuing the MRA, with or without replacement medication;
4. Agrees to comply with the contraception and reproduction restrictions of the study as follows:
   Male subjects must agree to abstain from sperm donation from Day 1 through 90 days after the final dose of study drug;
   Postmenopausal women must have had no menstrual bleeding for at least 1 year and either be >60 years or have an elevated plasma follicle-stimulating hormone (FSH) level >40 mIU/mL at Screening;
   Female patients of childbearing potential (i.e., ovulating, pre-menopausal, and not surgically sterile) must have a documented negative pregnancy test at Screening and Randomization;
   All male patients (unless surgically sterile) must use a highly effective method of contraception (i.e., <10% failure rate) from Day 1 through 90 days after the last administration of study drug;

Acceptable methods of contraception for male patients enrolled in the study include the following:
Condoms with spermicide; or
Surgical sterilization (vasectomy) at least 26 weeks before Screening; and
Female patients of childbearing potential must use a highly effective method of contraception (i.e., <1% failure rate) from Day 1 through 30 days after the last administration of study drug;

Acceptable methods of contraception for female patients enrolled in the study include the following:
Surgical sterilization (tubal ligation);
Intrauterine device for at least 12 weeks before Screening;
Hormonal contraception (oral, implant, injection, ring, or patch) for at least 12 weeks before Screening; or
Diaphragm used in combination with spermicide; and
5. Is able and willing to give informed consent for participation in the clinical study.

### (ii) Exclusion Criteria

Patients who meet any of the following criteria will be excluded from participation in the study:
1. Has a mean seated SBP 180 mmHg or DBP 110 mmHg Mean seated BP is defined as the average of 3 seated BP measurements at any single clinical site visit. If the patient did not take their regularly scheduled antihypertensive medications prior to the visit (Visits 1, 3, or 4), 1 BP re-test is allowed within 2 days after taking the medications.
2. Has a body mass index (BMI) >45 kg/m² at Screening (provided they still satisfy the arm circumference requirements, see exclusion criteria #3);
3. Has an upper arm circumference <7 or >17 inches at Screening;
4. Has been on night shifts at any time during the 4 weeks before Screening;
5. Is using a beta blocker for any primary indication other than systemic hypertension (e.g., migraine headache);
6. Is not willing or not able to discontinue an MRA or a potassium sparing diuretic as part of an existing antihypertensive regimen;
7. Is not willing to discontinue taking a potassium supplement;
8. Is expected to receive or is receiving any of the exclusionary drugs (strong cytochrome P450 3A inducers and/or chronic use of non-steroidal anti-inflammatory drugs [NSAIDs])—patients using chronic NSAIDs at screening who are willing to come off during the course of the study, are allowed to participate;
9. Has known secondary causes of hypertension (e.g., renal artery stenosis, uncontrolled or untreated hyperthyroidism, uncontrolled or untreated hypothyroidism, hyperparathyroidism, pheochromocytoma, Cushing's syndrome, or aortic coarctation) except obstructive sleep apnea; Patients with primary aldosteronism CAN BE considered for enrollment unless an adrenalectomy is expected before the end of their participation in the study.
10. Has documented estimated glomerular filtration rate <45 mL/min/1.73 m² using the Chronic Kidney Disease Epidemiology Collaboration equation at Screening;²¹
11. Has known and documented New York Heart Association stage III or IV chronic heart failure at Screening;
12. Has had a stroke, transient ischemic attack, hypertensive encephalopathy, acute coronary syndrome, or hospitalization for heart failure within 6 months before Screening;
13. Has known current severe left ventricular outflow obstruction, such as obstructive hypertrophic cardiomyopathy and/or severe aortic valvular disease diagnosed from a prior echocardiogram;
14. Has planned coronary revascularization (PCI or CABG) or any major surgical procedure;
15. Has had CABG or other major cardiac surgery (e.g., valve replacement), peripheral arterial bypass surgery, or PCI within 6 months before Screening;
16. Has chronic permanent atrial fibrillation;
17. Has uncontrolled diabetes with HbA1c>9.5% at Screening;
18. Has planned dialysis or kidney transplant during the course of this study;
19. Has had prior solid organ transplant and/or cell transplants;
20. Has known hypersensitivity to (R)-Compound 1 or drugs of the same class, or any of its excipients;
21. Has any clinically relevant medical or surgical conditions (including patients with unstable conditions and/or treated with systemic immunosuppressants including corticosteroids) that would put the patient at risk by participating in the study;
22. Has evidence of the following at Screening or at the start of the SB-RI Period (1 retest is allowed):
   White blood cell count >15×10⁹/L or absolute neutrophil count <1×10⁹/L;
   Potassium <3.5 mEq/L;
   Potassium >5.0 mEq/L;
   Hemoglobin <10.0 g/dL and/or anticipated initiation of erythropoietin-stimulating agents and/or planned transfusion within 2 months after Screening; or
   Serum aspartate aminotransferase and/or alanine aminotransferase >3×the upper limit of normal range, with a corresponding bilirubin >2 mg/dL, unless patient has a history of Gilbert's syndrome;
23. Is positive for HIV antibody, HCV RNA, or HBsAg;
24. Has typical consumption of 14 alcoholic drinks weekly. One drink of alcohol is equivalent to 12 pint of beer (285 mL), 1 glass of spirits (25 mL), or 1 glass of wine (125 mL);
25. Is pregnant, breastfeeding, or planning to become pregnant during the study;
26. Has participated in another clinical study involving any investigational drug within 30 days prior to Screening, or plans to participate in another clinical study within 30 days of discontinuation of study drug;
27. Has received experimental therapy with a small molecule within 30 days of Day 1 or 5 half-lives, whichever is greater, or received experimental therapy with a large molecule within 90 days of Day 1 or 5 half-lives, whichever is greater; or
28. Is considered to be unsuitable for any other reason that may either place the patient at increased risk during participation or interfere with the interpretation of the study outcomes, after reviewing medical and psychiatric history, physical examination, and laboratory evaluation.
   (iii) Randomization Criteria

Patients must meet all of the following criteria at Randomization (Visit 4):
1. Continues to satisfy all Inclusion/Exclusion Criteria;
2. Has no change in background therapy consisting of 3 antihypertensive medications for at least 4 weeks prior to Randomization;
3. Has 70% and ≤120% adherence to each antihypertensive medication and placebo during the SB-RI Period, based on pill counts on the morning of Randomization; and
4. Has a mean (the average of 3 measurements) seated BP 130/80 mmHg at Randomization.

### (iv) Withdrawal Criteria

Participation of patients in this clinical study will be discontinued for any of the following reasons:
The patient withdraws consent or requests discontinuation from the study for any reason;
The patient has mean seated BP>175/105 mmHg at 2 separate occasions during the Double-Blind Treatment Period;
The patient has occurrence of any medical condition or circumstance that exposes the patient to substantial risk and/or does not allow the patient to adhere to the requirements of the protocol;
The patient has any SAE, clinically significant AE, severe laboratory abnormality, intercurrent illness, or other medical condition which indicates that continued participation is not in the best interest of the patient;
The patient has a requirement of prohibited concomitant medication;
The patient fails to comply with protocol requirements or study-related procedures;
The patient becomes pregnant; or
The study is terminated.

If a patient withdraws prematurely from the study due to the above criteria or any other reason, they will be requested to undergo the Early Termination procedures and site staff should make every effort to complete the full panel of assessments scheduled for the End of Treatment (Visit 11). The reason for patient withdrawal must be documented. Patients should still attend study visits after Early Termination for safety monitoring.

### Criteria for Temporary Suspension of Dosing

Dosing of patients in this clinical study may be suspended temporarily for any of the following reasons.
Any SAE that is deemed related to the study drug, including death;
Withdrawal of a patient after randomization and who has received one or more doses of study drug for safety-related reasons;
A study drug-related AE deemed to be severe in intensity (severity) in ≥2 patients;
A study drug-related AE from a single System Organ Class deemed to be of moderate intensity (severity) in ≥4 patients; or
Potassium ≥6 mEq/L; the patient should stop study drug dosing and present to the clinical site immediately for repeat testing. Note: This criterion is specific only to the individual subject suspending dosing. The patient may restart study drug following consultation and approval from the Medical Monitor.

When the below event occurs, it should be reported as soon as possible:
The patient has evidence of hyponatremia (sodium concentrations <130 mmol/L with repeat confirmation within 72 hours upon notification) or SBP≤90 mmHg with symptoms consistent with postural hypotension; This criterion is specific only to the individual subject suspending dosing. Other enrolled subjects do not need to suspend dosing. The patient may restart study drug following approval.

### Study Treatments

### (i) Treatment Groups

Eligible patients will be randomized in a 1:1:1 ratio to one of the following groups:
1 mg (R)-Compound 1;
2 mg (R)-Compound 1; or
Placebo.

The next dose level to be studied. will be 0.5 mg QD. Following review, Part A will enroll patients using a randomization plan to allow for approximately equal distribution between the following treatment groups at the conclusion of the study:
1 mg (R)-Compound 1;
2 mg (R)-Compound 1;
0.5 mg (R)-Compound 1;
Placebo.

### (ii) Single Blind-Run in Period

Placebo tablets, indistinguishable from the (R)-Compound 1 tablets, will be administered during the SB-RI Period to determine whether medication adherence is a factor in patients not achieving goal BP. The single-blind placebo will be included with the ongoing stable antihypertensive regimen.

(iii) Randomization and Double-Blind Treatment Period

Patients who meet all eligibility criteria will be randomized in a 1:1:1 ratio into 1 of the 3 treatment groups (2 active [1 mg and 2 mg (R)-Compound 1] and 1 placebo) for Part A. After approximately the first 25 randomized patients per group reach approximately 4 weeks of study drug dosing in the Double-Blind Treatment Period, emerging data will be evaluated and reported on cumulative SAEs collected during the study. Based on assessments, the next dose level of (R)-Compound 1 to be studied will be 0.5 mg QD.

Part A will enroll patients using a randomization plan to allow for approximately equal distribution between the treatment groups at the conclusion of the study.

Patients will be stratified according to their baseline SBP (<145 or ≥145 mmHg) and their baseline glomerular filtration rate (<60 or ≥60 mL/min/1.73 m²).

Following randomization, study drug will be dispensed in a double-blind manner. Randomization information will be concealed until the end of the study, with the exception of an emergency situation involving a patient that requires unblinding of the treatment assignment.

### (iv) (R)-Compound 1 Supply

### Formulation

(R)-Compound 1 tablets will be provided in the following strengths: 0.5 mg, 1 mg and 2 mg. The tablets will be packaged in blister packs to achieve the doses required for the study. (R)-Compound 1 tablets will contain the study drug as the active ingredient and lactose anhydrous, microcrystalline cellulose, croscarmellose sodium, colloidal silicon dioxide, and magnesium stearate as inactive ingredients.

Matching placebo tablets will contain no active ingredient and the same inactive ingredients.

### Study Drug Administration

Patients will be allowed a normal diet every morning of study drug administration. On clinic visit days, patients will self-administer the morning dose of background hypertensive medications at home and withhold the morning dose of study drug.

Patients will self-administer the morning dose of study drug at the clinic to be witnessed by site staff after completion of pre-dose evaluations and laboratory sampling.

Patients participating in Part B will present to the clinical site at Visit 11 in a fasted state for 8 hours relative to study drug administration and will remain so for 4 hours after study drug administration. Patients will not be able to eat or drink other than water during the 12 hours of fasting.

### Treatment Adherence

Patients will self-administer the morning dose of study drug at the clinic to be witnessed by site staff during all clinic visits.

For all protocol-specified doses when the patient is not at the clinical site, patients will self-administer study drug at home and continue taking their background antihypertensive medications.

Excluded Medications and/or Procedures

Use of the following investigational, prescription, or over-the-counter medications is not permitted during the study:
Strong CYP3A inducers such as those in Table 18.

TABLE 18

| Group | Excluded Medications |
|---|---|
| Strong CYP3A inducers¹ | Apalutamide, carbamazepine², |
| | enzalutamide³, mitotane, |
| | phenytoin⁴, rifampin⁵, St. John's wort⁶ |

¹Examples of clinical inducers for P450-mediated metabolisms (for concomitant use clinical DDI studies and/or drug labeling). Strong, moderate, and weak inducers are drugs that decreasethe AUC of sensitive index substrates of a given metabolic pathway by ³80%, ³50% to <80%, and ³20% to <50%, respectively.
²Strong inducer of CYP2B6, CYP3A, and weak inducer of CYP2C9.
³Strong inducer of CYP3A and moderate inducer of CYP2C9, and CYP2C19.
⁴Strong inducer of CYP2C19, CYP3A, and moderate inducer of CYP1A2, CYP2B6, CYP2C8, CYP2C9.
⁵Strong inducer of CYP3A and moderate inducer of CYP1A2, CYP2C19. 6. Theeffect of St. John's wort varies widely and is preparation dependent.

Beta blockers for any primary indication other than systemic hypertension;
MRAs;
Chronic use of NSAIDs;

Patients using chronic NSAIDs at screening who are willing to come off during the course of the study, are allowed to participate
Potassium sparing diuretics; and/or
Potassium supplements.

### Study Procedures

### (i) Informed Consent

Written consent will be obtained from all patients before any protocol-specific procedures are performed.

Patients participating in the optional Part B sub-study at Visit 11 will first take part in the prior visits and procedures of Part A.

Patients who provide written informed consent to participate in the optional Part B sub-study will present to the clinical site at Visit 11 in a fasted state for 8 hours relative to study drug administration and will remain so for 4 hours after study drug administration. Patients will not be able to eat or drink other than water during the 12 hours of fasting. Post-dose PK blood sampling will be performed at the following timepoints at Visit 11: 1, 2, 3, 4, 6, and 8 hours. A ±5 minutes window is permitted for the collection of post-dose PK samples.

In some situations, collection of specific individual PK samples (including but not limited to the collection of the sample at 8 hours post-dose) may not be required.

### (ii) Early Termination Visit and Withdrawal Procedures

The End of Treatment for patients completing the study is Visit 11. For patients who are withdrawn from the study prior to completion, all Visit 11 procedures will be performed at the Early Termination Visit.

### Efficacy Assessments

### (i) Primary Efficacy Endpoint

The primary efficacy endpoint is the change from baseline in mean seated SBP after 12 weeks of treatment in patients with rHTN.

### (ii) Secondary Efficacy Endpoints

The secondary efficacy endpoints include the following:
Change from baseline in mean seated DBP of (R)-Compound 1 compared to placebo after 12 weeks of treatment in patients with rHTN; and
Percentage of patients achieving a seated BP response <130/80 mmHg of (R)-Compound 1 compared to placebo after 12 weeks of treatment for rHTN.

### Pharmacokinetic, Pharmacodynamic, and Pharmacogenomic Assessments

### (i) Pharmacokinetic Assessments

### Part A

Blood samples for PK analyses will be collected pre-dose at Visits 8 and 11 as specified in Table 17. Additional PK samples may also be collected in the event of an SAE, AE leading to withdrawal, or any other safety event. PK samples should be collected within approximately 15 minutes prior to dosing.

Samples will be analyzed to measure the plasma concentrations of (R)-Compound 1 and any measured metabolite(s) using validated liquid chromatography mass spectrometry methods.

### Part B

The date and time of the study drug taken at the clinical site on the day of Visit 11 will be recorded. The actual date and time of collection of each post-dose PK sample will also be recorded.

Patients in the optional Part B sub-study will have additional post-dose PK sampling performed at the following timepoints at Visit 11: 1, 2, 3, 4, 6, and 8 hours. A ±5 minutes window is permitted for the collection of post-dose PK samples.

The following plasma PK parameters will be determined for (R)-Compound 1 and any measured metabolite(s) using concentration data from the End of Treatment Visit (Visit 11), as the data permit:
Cₘₐₓ;
Tₘₐₓ; and
AUC from time 0 to the time of last measured plasma concentration.

### (ii) Pharmacodynamic Assessments

Blood samples for PD analyses will be collected pre-dose at Visits 4, 7, 8, and 11 as specified in Table 17. Urine samples from a 24-hour urine collection will be obtained over the 24 hours leading up to Visit 4 and 11 as specified in Table 17.

Plasma PD variables may include, but are not limited to, the following:
Aldosterone and its precursors (18-hydroxycorticosterone, corticosterone, and 11-deoxycorticosterone);
PRA; and
Cortisol (total) and its precursor 11-deoxycortisol.

Measurement of free cortisol will be performed if changes are noted in total cortisol.

Levels of plasma electrolytes (collected as part of the standard safety chemistry panel, see Table 19) will be used in the PD analysis.

TABLE 19
Clinical Laboratory Analytes
Standard Safety Chemistry Panel

| | |
|---|---|
| Alanine aminotransferase | Albumin |
| Alkaline phosphatase | Amylase |
| Aspartate aminotransferase | Bicarbonate |
| Blood urea nitrogen | Calcium |
| Chloride | Creatine kinase |
| Creatinine | Estimated glomerular filtration rate |
| Gamma-glutamyl transferase | Glucose |
| Inorganic phosphorus | Lactate dehydrogenase |
| Lipase | Potassium |
| Sodium | Total bilirubin |
| Total protein | Uric acid |

Additional Chemistry Parameter

Glycosylated hemoglobin

Hematology

| | |
|---|---|
| Hematocrit | Hemoglobin |
| Platelets | Red blood cell count |
| White blood cell count and differential¹ | |

Coagulation

Prothrombin time

Urinalysis

| | |
|---|---|
| Bilirubin | Blood |
| Glucose | Ketones |
| Leukocyte esterase | Microscopy ² |
| Nitrite | pH |
| Protein | Specific gravity |
| Urobilinogen | |

Endocrinology

| | |
|---|---|
| β-human chorionic gonadotropin ³ | Follicle-stimulating hormone (FSH) ⁴ |
| Serology | |
| Hepatitis B surface antigen | Hepatitis C virus RNA |
| HIV antibody | |

Pharmacodynamic Analytes

| | |
|---|---|
| Aldosterone and its precursors | Cortisol⁵ and its precursor 11-deoxycortisol |
| (18-hydroxycorticosterone, | |
| corticosterone, and 11- | |
| deoxycorticosterone) | |
| Plasma renin activity | B type Natriuretic Peptide |
| Pharmacokinetic Analytes | |
| (R)-Compound 1 | Any measured metabolite(s) of (R)-Compound 1 |

24-hour Urine Collection Analytes

| | |
|---|---|
| Aldosterone | Creatinine |
| Potassium | Albumin |
| Sodium | Protein |

¹Manual microscopic review is performed only if white blood cell count and/or differential values are out of reference range.
² Microscopy is performed only as needed based on positive dipstick test results.
³ Serum or point-of-care pregnancy tests will be performed only for female patients of childbearing potential (ovulating, pre-menopausal, and not surgically sterile).
⁴ FSH levels will be measured only for female patients who are post-menopausal for at least 1 year at Screening and are not surgically sterile.
⁵ Total cortisol will be measured. Measurement of free cortisol will be performed if changes are noted in total cortisol.

Urinary aldosterone and urine electrolyte levels will also be assessed from the 24-hour urine collections prior to Visits 4 and 11. Urine electrolyte levels may include, but not be limited to, urinary sodium and potassium (see Table 18).

PD samples will be collected in the morning at the clinical site, after the patient has been out of bed for approximately 2 hours and has been seated for 5 to 15 minutes. Samples will be analyzed using validated methods, as appropriate.

(iii) Pharmacogenomic Assessments

A single, optional, pharmacogenomic blood sample may be collected at any time after Randomization. The pharmacogenomic samples may be used for genetic research to explore the underlying causes of variability and/or differences in response in PK, PD, and/or safety data following administration of (R)-Compound 1.

Patients will be given the option to participate in the pharmacogenomic assessment during the consenting process. For patients who provide written informed consent to participate in the optional pharmacogenomic assessment, a blood sample will be collected at any time after Randomization. The patient may withdraw consent to participate in the pharmacogenomic assessment at any time during the study without withdrawing consent to participate in the study.

### Safety Assessments

### (i) Safety Endpoints

The safety of (R)-Compound 1 will be assessed from the time of informed consent until the end of the Follow-up Period. All safety endpoints will be summarized descriptively.

The safety endpoints will include the following:
Vital signs, standing BP and heart rate, physical examinations, electrocardiography, weight measurement, and clinical laboratory evaluations, including standard safety chemistry panel, hematology, coagulation, and urinalysis;
TEAEs;
Treatment-emergent SAEs;
TEAEs leading to premature discontinuation of study drug;
Treatment-emergent marked laboratory abnormalities; and
Change in mean standing SBP and DBP (measured pre-dose at the clinical site) from baseline to End of Treatment (Visit 11).

### (ii) Adverse Events

An AE is defined as any untoward medical occurrence in a clinical investigation that occurs to a patient administered a pharmaceutical product, which does not necessarily have a causal relationship with this treatment. An AE can therefore be any unfavorable and/or unintended sign (including an abnormal laboratory finding), symptom, or disease temporally associated with the use of a study drug, whether or not related to the study drug. All AEs, including observed or volunteered problems, complaints, or symptoms, are to be recorded. Clinical sites will record the time of event (hour, min) for AEs that start and/or end on the first randomized study drug administration visit (Visit 4) or at Visit 11 (EOT).

AEs, which include clinical laboratory test variables, will be monitored and documented from the time of informed consent until the end of the Follow-up Period. Patients should be instructed to report any AE that they experience, whether or not they think the event is due to study drug. Beginning at Screening, assessment for AEs should be made at each visit.

Wherever possible, a specific disease or syndrome, rather than individual associated signs and symptoms, should be identified. However, if an observed or reported sign or symptom is not considered a component of a specific disease or syndrome, it should be recorded. Additionally, the condition that led to a medical or surgical procedure (e.g., surgery, endoscopy, tooth extraction, or transfusion) should be recorded as an AE, not the procedure itself.

Any medical condition already present at Screening should be recorded as medical history and not be reported as an AE unless the medical condition or signs or symptoms present at baseline changes in severity, frequency, or seriousness at any time during the study. In this case, it should be reported as an AE.

Clinically significant abnormal laboratory or other examination (e.g., ECG) findings that are detected during the study or are present at Screening and significantly worsen during the study should be reported as AEs, as described below. Clinically significant abnormal laboratory values occurring during the clinical study will be followed until repeat tests return to normal, stabilize, or are no longer clinically significant. Abnormal test results that are determined to be an error should not be reported as an AE. Laboratory abnormalities or other abnormal clinical findings (e.g., ECG abnormalities) should be reported as an AE if any of the following are applicable:
If an intervention is required as a result of the abnormality; or
If action taken with the study drug is required as a result of the abnormality

### Adverse Drug Reaction

All noxious and unintended responses to a study drug related to any dose should be considered an adverse drug reaction. “Responses” to a study drug means that a causal relationship between a study drug and an AE is at least a reasonable possibility, i.e., the relationship cannot be ruled out.

### Unexpected Adverse Drug Reaction

An Unexpected Adverse Drug Reaction is defined as an adverse reaction, the nature or severity of which is not consistent with the applicable product information.

### Assessment of Adverse Events

The severity (intensity) of each AE will be assessed as mild, moderate, or severe, and will also categorize each AE as to its potential relationship to study drug using the categories of yes or no.

### Assessment of Severity

Mild—An event that is easily tolerated and generally not interfering with normal daily activities. Moderate—An event that is sufficiently discomforting to interfere with normal daily activities. Severe—An event that is incapacitating with inability to work or perform normal daily activities.

### Causality Assessment

The relationship of an AE to the administration of the study drug is to be assessed according to the following definitions:

No (not related, unlikely to be related)—The time course between the administration of study drug and the occurrence or worsening of the AE rules out a causal relationship and another cause (concomitant drugs, therapies, complications, etc.) is suspected.

Yes (possibly, probably, or definitely related)—The time course between the administration of study drug and the occurrence or worsening of the AE is consistent with a causal relationship and no other cause (concomitant drugs, therapies, complications, etc.) can be identified.

The definition implies a reasonable possibility of a causal relationship between the event and the study drug. This means that there are facts (evidence) or arguments to suggest a causal relationship.

The following factors should also be considered:
The temporal sequence from study drug administration. The event should occur after the study drug is given. The length of time from study drug exposure to event should be evaluated in the clinical context of the event.
Underlying, concomitant, intercurrent diseases. Each report should be evaluated in the context of the natural history and course of the disease being treated and any other disease the patient may have.
Concomitant drug. The other drugs the patient is taking or the treatment the patient receives should be examined to determine whether any of them might be recognized to cause the event in question.
Known response pattern for this class of study drug. Clinical and/or preclinical data may indicate whether a particular response is likely to be a class effect.
Exposure to physical and/or mental stresses. The exposure to stress might induce adverse changes in the recipient and provide a logical and better explanation for the event.
The pharmacology and PK of the study drug. The known pharmacologic properties (absorption, distribution, metabolism, and excretion) of the study drug should be considered.

### Adverse Events of Special Interest

Each patient will be monitored for clinical and laboratory evidence for predefined adverse events of special interest (AESIs) throughout the patient's participation in this study.

Any additional information on the AESI will be assessed in detail.

For this study, AESIs include the following:
Events of hypotension that require clinical intervention;
Sodium levels that require clinical intervention; or
Potassium levels that require clinical intervention.

AESIs must be recorded.

(iii) Serious Adverse Events

An AE or adverse reaction is considered serious if it results in any of the following outcomes:
Death;
A life-threatening AE. An AE or adverse reaction is considered “life-threatening” if its occurrence places the patient at immediate risk of death. It does not include an event that, had it occurred in a more severe form, might have caused death;
Requires hospitalization or prolongation of existing hospitalizations;

Any hospital admission with at least 1 overnight stay will be considered an inpatient hospitalization. An emergency room or urgent care visit without hospital admission will not be recorded as an SAE under this criterion, nor will hospitalization for a procedure scheduled or planned before signing of informed consent, or elective treatment of a pre-existing condition that did not worsen from baseline. However, unexpected complications and/or prolongation of hospitalization that occur during elective surgery should be recorded as AEs and assessed for seriousness. Admission to the hospital for social or situational reasons (i.e., no place to stay, live too far away to come for hospital visits, respite care) will not be considered inpatient hospitalizations;
A persistent or significant disability/incapacity or substantial disruption of the ability to conduct normal life functions;
A congenital anomaly/birth defect; or
An important medical event. Important medical events that do not meet any of the above criteria may be considered an SAE when, based upon appropriate medical judgment, they may jeopardize the patient and may require medical or surgical intervention to prevent 1 of the outcomes listed above. Examples of such medical events include allergic bronchospasm requiring intensive treatment in an emergency room or at home, blood dyscrasias or convulsions that do not result in inpatient hospitalizations, or the development of drug dependency.

### (iv) Overdose Reporting

Overdose refers to the administration of a quantity of the study drug given per administration or cumulatively (accidentally or intentionally), which is above the maximum recommended dose according to the protocol.

In cases of a discrepancy in the drug accountability, overdose will be established only when it is clear that the patient has taken additional dose(s), or it is suspected that the patient has taken additional dose(s).

### (v) Safety Surveillance and Management of Potassium Levels

Serum potassium levels will be monitored systemically throughout the study. Potassium will be measured at the Central Laboratory at each visit as indicated in Table 17). Unscheduled assessments of potassium levels should be completed as needed for acute management of the patient (e.g., follow-up from elevatedcentral lab potassium, acute changes in clinical condition, suspected dehydration, etc.).

For serum potassium ≥5.5 mEq/L and <6 mEq/L, the patient should present to the clinical site immediately for repeat testing, but study drug dosing may continue.

For serum potassium ≥6 mEq/L, the patient should suspend study drug dosing and present to the clinical site immediately for repeat testing.

### (vi) Clinical Laboratory Evaluations

Blood samples for standard safety chemistry panel, hematology, and coagulation will be obtained and assessed as indicated in Table 17. PD samples will be obtained as indicated in Table 17 and assessed. PK samples will be obtained as indicated in Table 17 and assessed. See Table 18 for a complete list of analytes.

A serum or POC pregnancy test will be performed for female patients of childbearing potential as indicated in Table 17.

Urine samples (including samples from 24-hour urine collection) will be obtained as indicated in Table 17 and assessed at the Central Laboratory per institutional guidelines for complete urinalysis.

Blood samples for pharmacogenomic assessments will be stored and analyzed at Cincinnati Children's Hospital Medical Center.

Screening laboratory evaluations, if abnormal, may be repeated once for eligibility purposes.

(vii) Vital Signs and Blood Pressure Measurement

Vital signs will include heart rate, respiratory rate, and body temperature. Orthostatic vitals will include standing BP and standing heart rate. Vitals signs and BP will be measured at visits as indicated in Table 17 using the following standardized procedures:
Patients should not exercise, smoke, or consume caffeinated beverages or food 30 minutes prior to assessment of vital signs and AOBPM;
On visits when study drug will be administered, vital signs and BP will be assessed pre-dose;
Vital signs and BP measurements should be obtained prior to ECG recordings; and
For measuring BP by AOBPM, the following additional standardized procedures are recommended:
   Patient should be seated for at least 5 minutes in the examination room with the back supported, feet flat on the floor, and the measurement arm supported so that the midpoint of the manometer cuff is at heart level;
   A designated AOBPM device will be provided to each clinical site and must be used for all study-related measurements;
   An appropriately sized cuff should be used with the bladder centered over the brachial artery;
   The cuff size and arm used for the measurement should be recorded;
   The arm with the higher mean BP value at Screening should be used for Screening and subsequent BP measurements;
   All BP measurements should be obtained at approximately the same time of day as the Screening measurements are obtained.
   3 seated BP measurements (each measurement 1 to 2 minutes apart) should be obtained using the same arm and the AOBPM device at each clinical site visit. Mean seated BP is defined as the average of 3 seated BP measurements at any single clinical site visit;
   If the lowest and highest SBP measurements are >15 mmHg apart, additional readings should be performed; and
   Once the seated BP has been determined, the patient will be asked to stand and after 60 seconds a single standing BP and heart rate measurement will be obtained, as required.
      (viii) Electrocardiograms

Standard 12-lead ECGs will be performed at Visits 1, 4, and 11 as indicated in Table 17. ECGs will be performed after the patient has been resting in the supine position for at least 10 minutes. 12-lead ECGs will be printed and will be interpreted as soon as possible. All ECGs collected at the time of Randomization, End of Treatment, and Early Termination Visits must be evaluated for the presence of abnormalities. Standard ECG parameters will be measured, and the following ECG parameters will be recorded:
QRS interval;
Heart rate;
RR interval;
QT interval; and
QTc (QTcF).

Clinically meaningful changes from baseline electrocardiograms, include but are not limited to:
QTcF≥450 msec (male);
QTcF≥470 msec (female);
A >60 msec increase in QTcF from baseline; or
A ≥6% increase in QTcF from baseline.

New onset findings including, but not limited to, the following:
Second degree atrioventricular (AV) block (Mobitz II);
Third degree AV block (complete heart block);
Acute myocardial infarction;
New left bundle branch block;
Severe bradycardia (ventricular rate ≤40 bpm);
Supraventricular tachycardia (ventricular rate ≥150 bpm);
Torsades de pointes;
Ventricular tachycardia (≥3 beats regardless of rate);
Ventricular fibrillation; or
Atrial fibrillation/atrial flutter (ventricular rate ≥150 bpm).

### (ix) Physical Examinations

A complete physical examination will include assessment of general appearance, skin, head, eyes, ears, mouth, oropharynx, neck, heart, lungs, abdomen, extremities, and neuromuscular system and will be performed at Visits 1 and 11 as indicated in Table 17.

A limited physical examination will consist of a minimum of general appearance, skin, heart, lungs, and abdomen and will be performed at the other clinical site visits.

### (x) Height and Weight

Weight will be measured at the visits indicated in Table 17. Height measured at Visit 1 will be used to calculate BMI at subsequent visits. Height will be measured with the patient's shoes off Weight will be measured with the patient's shoes off and after the patient's bladder has been emptied.

### Statistics

### (i) Analysis Populations

Intent-to-Treat Population (ITT): The ITT Population will include all patients randomized into the study. Treatment classification will be based on the randomized treatment.

Modified Intent-to-Treat Population (mITT): The mITT Population will include all patients in the ITT Population who receive at least 1 dose of any study drug and have a baseline value for the SBP assessment. Any efficacy measurement obtained after a patient received a restricted BP altering therapy, outside of the current study design, will be removed from the mITT analysis. Treatment classification will be based on the randomized treatment. The mITT Population will be used for the primary analysis of all efficacy endpoints.

Per-Protocol Population (PP): The PP Population will include all patients in the mITT Population who have a baseline value for the SBP assessment, have an End of Treatment Visit (Visit 11) value for the SBP assessment, and who did not experience a major protocol deviation that potentially impacted the primary efficacy endpoint. The PP Population, along with the reason for exclusion, will be finalized prior to study unblinding.

Safety Population: The Safety Population will include all patients who receive at least 1 dose of any randomized study drug. Treatment classification will be based on the actual treatment received. The Safety Population will be the primary population used for the safety analyses.

Pharmacokinetic Population: The PK Population will include all patients in the mITT Population who have at least 1 quantifiable plasma concentration.

Pharmacodynamic Population: The PD population will include all patients in the mITT Population who have at least 1 quantifiable concentration of a PD variable.

### (ii) Statistical Methods

All study-collected data will be summarized by treatment group using descriptive statistics, graphs, and/or raw data listings. Descriptive statistics for continuous variables will include number of patients (n), mean, standard deviation (SD), median, minimum, and maximum values. Analysis of categorical variables will include frequency and percentage.

### Efficacy Analysis

The PP Population will be the primary population for the efficacy analyses. Efficacy will also be analyzed using the ITT Population and the mITT Population as supportive analyses.

The primary efficacy analysis will compare the change in mean seated SBP from baseline (Visit 4) to End of Treatment (Visit 11) between each dose strength of (R)-Compound 1 and placebo. A mixed model for repeated measures will be used to perform this analysis. The analysis will include fixed effects for treatment, visit, and treatment-by-visit interaction, along with a covariate of the baseline value. The restricted maximum likelihood estimation approach will be used with an unstructured covariance matrix. The least squares means, standard errors, and 2-sided 95% confidence intervals for each treatment group and for pairwise comparisons of each dose strength of (R)-Compound 1 to the placebo group will be provided. To protect the overall alpha level on the primary endpoint, the hypothesis testing will be performed sequentially. The first comparison will be between the highest active dose group and placebo at the 2-sided alpha=0.05 level; if significant, the next highest active dose group will be compared to placebo at the 2-sided alpha=0.05 level. Hypothesis testing will proceed in this step down fashion until a comparison is not significant. At that point, all remaining sequential tests will be deemed not significant.

Missing data will be imputed using multiple imputation methodology. Results will be combined using Rubin's method.

Similar models will be used to analyze DBP and PD variables. Logistic regression analyses will be used to analyze binary endpoints with model covariates of treatment group, baseline SBP, and baseline DBP. No adjustment will be made for multiplicity in testing the secondary efficacy endpoints.

Safety Analysis: The Safety Population will be the primary population for the safety analysis. All safety endpoints will be summarized descriptively.

### Pharmacokinetic Analysis

Individual plasma concentration data for (R)-Compound 1 and any measured metabolite(s) will be listed and summarized by visit, timepoint, and treatment group for the PK Population.

For patients participating in Part B of the study, relevant parameters for (R)-Compound 1 and any measured metabolite(s) will be listed by individual patient and summarized by treatment for active treatments. Mean and individual plasma concentrations of (R)-Compound 1 and any measured metabolite(s) will be plotted against time points by regimen for patients in Part B.

Pharmacodynamic Analysis: The PD Population will be the primary population for the PD analysis. All PD variables will be summarized descriptively.

Pharmacokinetic-Pharmacodynamic Analysis: An attempt will be made to correlate plasma concentrations and parameters with measures of safety, PD, and/or efficacy, if the data permit.

Interim Analysis: A formal unblinded interim analysis may be performed based on previous review(s) of the safety data.

(iii) Sample Size Determination

### Part A

A sample size of at least 308 evaluable patients (i.e., 77 patients per treatment group) will provide >80% power to detect a 5 mmHg difference in mean seated SBP (SD=11 mmHg) after 12 weeks of treatment with 3 dose strengths of (R)-Compound 1 compared to placebo at a 2-sided significance level of 0.05.

The sample size for this study was determined in order to provide sufficient power for the analyses of the primary efficacy endpoint described above. Therefore, assuming an approximately 13% dropout rate, enrollment of approximately 348 patients (i.e., 87 patients per treatment group) is planned for this study.

Patients will be stratified according to their baseline SBP (<145 or ≥145 mmHg) and their baseline glomerular filtration rate (<60 or ≥60 mL/min/1.73 m²).

### Part B

Approximately 10-15% of the patients are expected to participate in this optional sub-study. The sample size was chosen empirically to support the stated objectives and without formal statistical considerations. The proposed sample size is considered adequate to characterize the PK of (R)-Compound 1 in patients with rHTN.

### Example 8

### Study Objectives:

The objectives of this study were as follows:
To assess the impact of Compound 1 on the pharmacokinetics (PK) of immediate-release metformin; and
To assess the safety and tolerability of coadministration of Compound 1 and metformin as compared to that of metformin alone.

### Methodology:

This was a randomized, open-label, two-period, crossover, Phase 1 study to assess the impact of Compound 1 on the PK of metformin and the safety and tolerability of coadministration of Compound 1 and metformin as compared to that of metformin alone. Up to 32 subjects were to be enrolled in the study with the intent that a minimum of 24 subjects would complete both treatment periods. Subjects were randomly assigned to 1 of 2 treatment sequences (AB or BA) below on Day 1 of Treatment Period 1:
Treatment A: a single 1000 mg dose of immediate-release metformin; and
Treatment B: a single 1000 mg dose of immediate-release metformin coadministered with a 10 mg dose of Compound 1.

Note: Metformin was administered 2 hours after a single 10 mg dose of Compound 1.

Subjects were administered study drug on the morning of Day 1 of each treatment period.

For each subject, the study consisted of the following:
A screening period of up to 26 days;
Two 4-day inpatient periods (from Check-In through completion of treatment), each consisting of a single dose of study drug (metformin alone or coadministered with Compound 1) followed by 3 days of PK sampling; and
A follow-up phone call 3 days (+1 day) after completion of Treatment Period 2.

There was a minimum 10-day washout between administration of study drug in each treatment period. Subjects were confined from Check-In on the day prior to dosing in each treatment period through collection of the final PK sample in each treatment period.

Safety was assessed throughout the study based on adverse events (AEs), physical examinations, weight measurements, electrocardiograms (ECGs), vital signs assessments (seated and orthostatic), and clinical laboratory evaluations.

Unscheduled procedures or visits and/or additional follow-up may have been required for subjects with clinically significant abnormal laboratory findings, unresolved treatment-emergent AEs (TEAEs), serious AEs (SAEs) that required follow-up laboratories and review, and clinically significant AEs.

### Duration of Treatment:

There were two 4-day inpatient periods (from Check-In through the completion of treatment), each consisting of a single dose of study drug (metformin alone or coadministered with Compound 1). There was a minimum 10-day washout between administration of study drug in each treatment period.

### Number of Subjects:

Planned: up to 32 subjects planned
Screened: 51 subjects screened
Randomized: 27 subjects randomized
Completed: 26 subjects completed
Discontinued: 1 subject discontinued from the study

### Diagnosis and Main Criteria for Inclusion:

The population for this study included healthy subjects between the ages of 18 and 55 years, inclusive, who had a body mass index (BMI) between 18 and 30 kg/m², inclusive; were in good health based on medical/surgical and psychiatric history, physical examination, ECG, vital signs (seated and orthostatic), and routine laboratory tests (serum chemistry, hematology, and urinalysis); had normal renal function; and were nonsmokers.

### Investigational Product and Comparator Information:

Compound 1 was supplied as 5 mg oral tablets. Immediate-release metformin was obtained from a commercial supplier as 500 mg oral tablets.

### Criteria for Evaluation:

### Pharmacokinetics:

The following plasma PK parameters were determined for Compound 1 and its primary metabolite (Compound 1-metabolite):
Maximum observed plasma concentration (Cₘₐₓ), determined directly from the concentration-time profile;
Time to Cₘₐₓ (Tₘₐₓ), defined as the first time point with the maximum value, if the maximum value occurred at more than 1 time point;
Apparent first-order terminal elimination rate constant calculated from a semi-logarithmic plot of the plasma concentration versus time curve (z), calculated by linear least squares (LS) regression analysis using points in the terminal logarithmic-linear phase;
Area under the concentration-time curve (AUC) from time 0 to 24 hours (AUC₀₋₂₄);
AUC from time 0 to 72 hours;
AUC from time 0 to infinity (AUC_{0-inf}) (Compound 1 only), calculated as AUC from time 0 to the time of the last quantifiable plasma concentration (AUC₀₋ₜ)+last quantifiable plasma concentration (Cₗₐₛₜ)/λ_{z}; and
Percent of AUC_{0-inf} extrapolated (AUC_{% extrap}) (Compound 1 only), represented as (1−AUC₀₋ₜ/AUC_{0-inf})×100.

The following plasma PK parameters were determined for metformin:
Cₘₐₓ, determined directly from the concentration-time profile;
Tₘₐₓ, defined as the first time point with the maximum value, if the maximum value occurred at more than 1 time point;
λ_{z}, calculated by linear LS regression analysis using points in the terminal logarithmic-linear phase;
AUC₀₋₂₄;
AUC₀₋ₜ;
AUC_{0-inf}, calculated as (AUC₀₋ₜ+Cₗₐₛₜ/λ_{z});
AUC_{% extrap}, represented as (1−AUC₀₋ₜ/AUC_{0-inf})×100; and
Terminal phase elimination half-life, calculated as ln(2)/λ_{z}.

The following urine PK parameters were determined for metformin:
Cumulative amount of metformin excreted in the urine (cumulative amount of drug excreted in the urine [Ae]);
Renal clearance, calculated as Ae/AUC; and
Fraction of the dose excreted renally, calculated as 100×Ae/Dose.

### Safety:

The following safety assessments were performed:
AEs and SAEs;
Clinical laboratory test assessments;
Vital signs including heart rate, blood pressure (BP) (including orthostatic BP when indicated), respiration rate, and temperature;
12-lead ECGs;
Physical examinations; and
Height, weight, and BMI.

### Statistical Methods:

### Analysis Populations:

The Safety Population consisted of all randomized subjects who received any study drug (Compound 1 or metformin).

The PK Population included all subjects who received any study drug (Compound 1 or metformin) and had at least 1 quantifiable postdose plasma concentration for Compound 1, metformin, or any measured metabolite.

The PK Evaluable Population included all subjects who received any study drug (Compound 1 or metformin) and had sufficient plasma concentration data to characterize at least 1 PK parameter of Compound 1, metformin, or any measured metabolite.

### Pharmacokinetic Analyses:

Plasma concentrations of Compound 1, its primary metabolite (Compound 1-metabolite), and metformin were listed by individual subject and summarized by treatment using descriptive statistics for the PK Evaluable Population. Compound 1, Compound 1-metabolite, and metformin plasma concentrations were plotted against time points by treatment (mean and individual). Mean concentrations were plotted against nominal sampling times, while individual concentrations were plotted against actual sampling times.

Urine concentrations of metformin were listed by individual subject and summarized by treatment using descriptive statistics for the PK Evaluable Population. Plots of Ae by time point and treatment (individual and mean) were also presented.

Plasma and urine PK parameters were determined using non-compartmental methods as appropriate. Parameters were listed by individual subject and summarized by treatment using descriptive statistics for the PK Population.

Logarithmic transformations of PK parameters of metformin were analyzed using a mixed model including terms for sequence, treatment group, and period as fixed effects, and subject nested within sequence as a random effect.

The PK parameters analyses were based on the PK Population.

### Safety:

Safety analyses were performed throughout the study based on the Safety Population. Safety was evaluated through assessments of AEs, physical examinations, ECGs, weight measurements, vital signs assessments (seated and orthostatic), and clinical laboratory evaluations.

TEAEs were summarized by Medical Dictionary for Regulatory Activities system organ class (SOC) and preferred term (PT) for each treatment and overall.

The Safety Population was used for all the safety analyses. The following summaries of AEs were presented for each part:
Overall summary of TEAEs by treatment and overall with a breakdown by AE maximum severity;
TEAEs by SOC and PT by treatment;
TEAEs by SOC, PT, and relationship to study drug or metformin by treatment;
TEAEs by SOC, PT, and maximum severity by treatment; and
TEAEs by SOC, PT, maximum severity, and the relationship to the study drug by treatment.

Separate listings were prepared for SAEs, AEs leading to death, and AEs leading to study discontinuation.

Safety laboratory values and changes from baseline were summarized descriptively by treatment group at each time point of collection, when appropriate. Shift tables describing out-of-normal range shifts were provided for clinical laboratory results. All safety laboratory data were provided in data listings.

Pregnancy test results, drug and alcohol testing results, and viral serology results were listed.

Vital signs; continuous ECG parameters; height, weight, and BMI values and changes form baseline were summarized descriptively by treatment group at each time point of collection. All data were listed.

Physical examination results by body system were summarized at each time point of collection by treatment group and findings were listed.

### Summary of Results

### Pharmacokinetics:

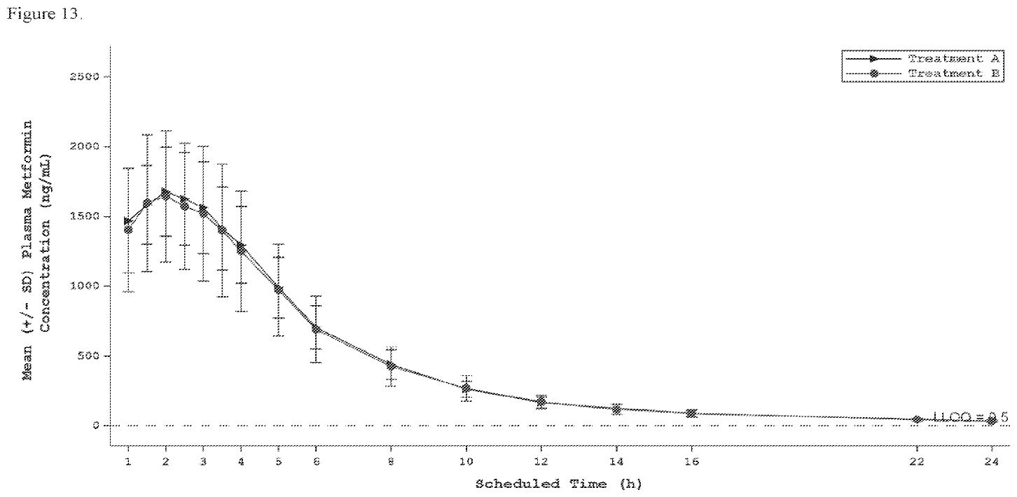

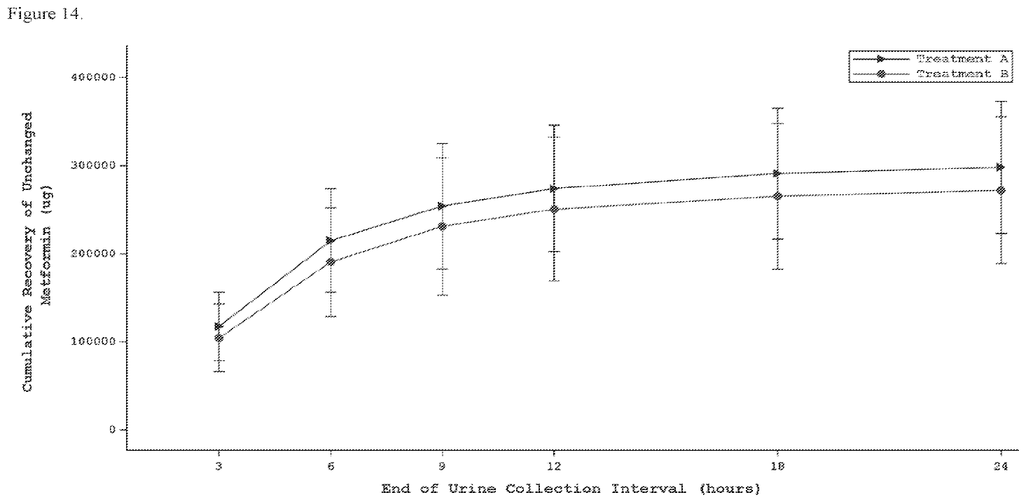

The plasma concentration-time course curves for metformin were nearly identical in the presence and absence of Compound 1. See FIGS. 13 and 14.

The ratios of geometric LS mean for Treatment B:Treatment A for metformin plasma Cₘₐₓ, AUC_{0-inf}, and AUC₀₋ₜ all approximated 100%, with 90% confidence interval values falling within the acceptance range of 80% to 125%, indicating that systemic exposure to metformin was not affected by Compound 1.

Consistent with the lack of observed effect of Compound 1, urinary excretion of metformin was qualitatively and quantitatively similar in the presence and absence of Compound 1.

### Safety:

The safety results of the current study indicate that metformin was well tolerated when administered alone or 2 hours after a single 10 mg dose of Compound 1. There were no deaths, SAEs, or discontinuations due to a TEAE, and there was no noteworthy increase in the incidence of AEs when metformin and Compound 1 were coadministered versus metformin administered alone. All TEAEs experienced by subjects were mild in severity. The most frequent TEAEs were gastrointestinal-related, as one would expect with a single high dose of metformin. There were no trends or clinically meaningful changes in laboratory parameters or physical examination results. There were no clinically significant changes observed in vital signs or 12-lead ECG findings, including no meaningful changes in QTcF.

### Conclusions

Metformin was well tolerated when administered alone or 2 hours after a dose of Compound 1. Compound 1 did not result in an increase in metformin plasma concentrations or a decrease in metformin renal clearance when compared with administration of metformin alone. Based on the results from this study, dose adjustment of metformin is not considered necessary when metformin is coadministered with Compound 1.

This was a randomized, open-label, two-period, crossover, Phase 1 study to assess the impact of Compound 1 on the PK of metformin and the safety and tolerability of coadministration of Compound 1 and metformin as compared to that of metformin alone. Up to 32 subjects were to be enrolled in the study with the intent that a minimum of 24 subjects would complete both treatment periods. Subjects were randomly assigned to 1 of 2 treatment sequences (AB or BA) below on Day 1 of Treatment Period 1:
Treatment A: a single 1000 mg dose of immediate-release metformin; and
Treatment B: a single 1000 mg dose of immediate-release metformin coadministered with a 10 mg dose of Compound 1.

Note: Metformin was administered 2 hours after a single 10 mg dose of Compound 1.

Subjects were administered study drug on the morning of Day 1 of each treatment period.

For each subject, the study consisted of the following:
A screening period of up to 26 days;
Two 4-day inpatient periods (from Check-In through completion of treatment), each consisting of a single dose of study drug (metformin alone or coadministered with Compound 1) followed by 3 days of PK sampling; and
A follow-up phone call 3 days (+1 day) after completion of Treatment Period 2.

There was a minimum 10-day washout between administration of study drug in each treatment period. Subjects were confined from Check-In on the day prior to dosing in each treatment period through collection of the final PK sample in each treatment period.

Safety was assessed throughout the study based on AEs, physical examinations, weight measurements, ECGs, vital signs assessments (seated and orthostatic), and clinical laboratory evaluations.

Unscheduled procedures or visits and/or additional follow-up may have been required for subjects with clinically significant abnormal laboratory findings, unresolved TEAEs, SAEs that required follow-up laboratories and review, and clinically significant AEs.

### Selection of Study Population

### Inclusion Criteria

Subjects who met all of the following criteria based on Screening and Check-In results (Treatment Period 1) were eligible to participate in the study:
1. Healthy subjects between the ages of 18 and 55 years, inclusive, at Screening;
2. Body mass index (BMI) between 18 and 30 kg/m², inclusive;
3. Good health based on medical/surgical and psychiatric history, physical examination, ECG, vital signs (seated and orthostatic), and routine laboratory tests (serum chemistry, hematology, and urinalysis);
4. Normal renal function, defined as estimated glomerular filtration rate 85 mL/min/1.73 m² at Screening and Day −1;
5. Nonsmokers who had not used nicotine-containing products (ie, cigarettes, nicotine patch, nicotine chewing gum, or electronic cigarettes) for at least 6 months prior to Screening;
6. Male subjects with female partners of childbearing potential must have agreed to use 2 medically accepted, highly effective methods of birth control from Day 1 through 90 days after administration of the final dose of study drug (per Section 5.6.2 of the Clinical Study Protocol [Appendix 16.1.1]);
7. Male subjects must have agreed to abstain from sperm donation from Day 1 through 90 days after administration of the final dose of study drug;
8. Female subjects with male partners must have been surgically sterile (hysterectomy and/or bilateral oophorectomy), postmenopausal for at least 1 year (with follicle-stimulating hormone in postmenopausal range), or have agreed to use 2 medically accepted, highly effective methods of birth control from Day −14 until 60 days following administration of the final dose of study drug (per Section 5.6.2 of the Clinical Study Protocol [Appendix 16.1.1]); and
9. Was able to understand and willing to comply with study procedures and restrictions (including confinement to the study site, fasting and meal requirements, and restrictions on physical activity, use of recreational drugs or alcohol, and medications) and provided written informed consent according to institutional and regulatory guidelines.

### Exclusion Criteria

Subjects who met any of the following criteria based on Screening and Check-In results were excluded from participation in the study:
1. Was actively participating in an experimental therapy study; received experimental therapy with a small molecule other than Compound 1 within 30 days of the first dose of study drug or 5 half-lives, whichever was longer; or received experimental therapy with a large molecule within 90 days of the first dose of study drug or 5 half-lives, whichever was longer;
2. Had a personal or family history of long QT syndrome, Torsades de Pointes, other complex ventricular arrhythmias, or family history of sudden death;
3. Had a history of, or current, clinically significant arrhythmias, as judged by the Investigator, including ventricular tachycardia, ventricular fibrillation, atrial fibrillation, sinus node dysfunction, or clinically significant heart block. Subjects with minor forms of ectopy (eg, premature atrial contractions) were not necessarily excluded;
4. Had prolonged QTcF (>450 msec);
5. Had seated systolic blood pressure (BP) >140 mmHg and/or diastolic BP>90 mmHg or systolic BP<90 mmHg and/or diastolic BP<50 mmHg;
6. Had a resting heart rate >100 or <50 bpm;
7. Had a body temperature >37.6° C. (99.68° F.), measured orally, or respiration rate <12 or >20 breaths/minute;
8. Had postural tachycardia (ie, >30 bpm upon standing) or orthostatic hypotension (ie, a fall in systolic BP of ≥20 mmHg or diastolic BP of ≥10 mmHg upon standing);
9. Had serum potassium >upper limit of normal (ULN) of the reference range and serum sodium <lower limit of normal of the reference range;
10. Had aspartate aminotransferase, alanine aminotransferase, or total bilirubin values >1.2×ULN;
11. Was positive for human immunodeficiency virus antibody, hepatitis C virus antibody, hepatitis B surface antigen, or SARS-CoV-2 RNA;
12. Had any other clinical laboratory values which were meaningfully outside of normal limits (based on laboratory normal range) in the opinion of the Investigator;
13. Had a known history of porphyria, myopathy, or active liver disease;
14. Had evidence or history of any clinically significant immunologic, hematologic, renal, endocrine, pulmonary, gastrointestinal, cardiovascular, musculoskeletal, hepatic, psychiatric, neurologic, or allergic (including clinically significant or multiple drug allergies) disease; surgical conditions; cancer (with the exception of basal or squamous cell carcinoma of the skin and cancer that had resolved or had been in remission for >5 years prior to Screening); or any condition that, in the Investigator's opinion, may have confounded study procedures or results, impacted subject safety, or interfered with the absorption, distribution, metabolism, or excretion of the study drug (appendectomy allowed, cholecystectomy prohibited);
15. Had evidence or history of acute or chronic metabolic acidosis, including diabetic ketoacidosis;
16. Had any prior episode(s) of lactic acidosis;
17. Had radiologic scan with contrast within 14 days prior to the first dose of study drug;
18. Used any prescription medications, including topicals or over-the-counter medications (other than occasional use of acetaminophen or nonsteroidal anti-inflammatory drugs, such as ibuprofen or naproxen, according to the package insert); herbal supplements; dietary supplements; or nutraceuticals within 14 days prior to the first dose of study drug or 5 half-lives, whichever was longer, or was unwilling to refrain from these medications through discharge from the study site; Note: Use of over-the-counter topical medications may have been permitted in consultation with the Sponsor. In addition, use of medications for which 5 half-lives exceeded 14 days must have been discussed with and approved by the Sponsor prior to subject enrollment.
19. Had corticosteroid use (systemic or extensive topical use) within 3 months (90 days) prior to the first dose of study drug;
20. Had a positive drug or alcohol test result at Screening or Check-In, or a history of alcoholism or drug abuse within 2 years prior to the first dose of study drug, as defined by the Diagnostic and Statistical Manual of Mental Disorders, 4th Edition;
21. Had typical consumption of ≥14 alcoholic drinks weekly;
   Note: 1 drink of alcohol was equivalent to ½ pint of beer (285 mL), 1 glass of spirits (25 mL), or 1 glass of wine (125 mL).
22. Had history or evidence of illicit drug use within the past 2 years;
23. Had surgical procedures within 4 weeks prior to Check-In (other than minor cosmetic surgery or minor dental procedures) or planned elective surgery during the treatment period;
24. Had any illness within 4 weeks prior to Check-In, unless deemed not clinically significant by the Investigator;
25. Had a known allergy to any ingredient of Compound 1 or metformin;
26. Had any history of severe allergic reaction (including drugs, food, insect bites, or environmental allergens);
27. Had inadequate venous access;
28. Was currently undergoing treatment with weight loss medication or had prior weight loss surgery (eg, gastric bypass surgery);
29. Was pregnant, breastfeeding, or planning to become pregnant during the study; or
30. Was considered by the Investigator, after reviewing medical and psychiatric history, physical examination, and laboratory evaluation, to be unsuitable for any other reason that may have either placed the subject at increased risk during participation or interfered with the interpretation of the study outcomes.
   Removal of Subjects from Therapy or Assessment

Participation of a subject in this study may have been discontinued for any of the following reasons:
The subject withdrew consent or requested discontinuation from the study for any reason;
Occurrence of any medical condition or circumstance that exposed the subject to substantial risk and/or did not allow the subject to adhere to the requirements of the protocol;
Any SAE, clinically significant AE, severe laboratory abnormality, intercurrent illness, or other medical condition which indicated to the Investigator that continued participation was not in the best interest of the subject;
Pregnancy;
Requirement of prohibited concomitant medication (after consultation with the Sponsor);
Subject failure to comply with protocol requirements or study-related procedures; or
Termination of the study by the Sponsor or the regulatory authority.

### Treatments

### Treatments Administered

Each subject received each treatment once during the study. All subjects were randomized once to receive 1 of the following in each period:
Treatment A: a single 1000 mg dose of immediate-release metformin; or
Treatment B: a single 1000 mg dose of immediate-release metformin coadministered with a 10 mg dose of Compound 1.

Note: Metformin was administered 2 hours after a single 10 mg dose of Compound 1.

### Identity of Investigational Products

Compound 1 tablets were provided at a strength of 5 mg and packaged in high-density polyethylene bottles. Compound 1 tablets contained the study drug as the active ingredient and lactose anhydrous, microcrystalline cellulose, croscarmellose sodium, colloidal silicon dioxide, and magnesium stearate as inactive ingredients.

Immediate-release metformin (500 mg) was obtained from a commercial supplier.

### Method of Assigning Subjects to Treatment Groups

Subjects were randomly assigned to 1 of 2 treatment sequences (AB or BA) on Day 1 of Treatment Period 1 according to a pre-generated randomization scheme.

### Selection of Doses in the Study

Data from the previously completed SAD and MAD studies suggest that therapeutic doses of Compound 1 ≤10 mg are expected in the intended patient populations. Results of nonclinical assessments indicated that Compound 1 is an inhibitor of the renal transporters MATE-1 and MATE2-K (in a non-time-dependent manner). As such, consistent with Food and Drug Administration (FDA) guidance on Assessment of Drug-Drug-Interactions, a single 10 mg dose of Compound 1 was assessed in the current study to maximize the potential of detecting an interaction. See US Department of Health and Human Services, Food and Drug Administration, Center for Drug Evaluation and Research (CDER). Clinical drug interaction studies—Cytochrome P450 enzyme and transporter-mediated drug interactions—Guidance for industry. January 2020.

Metformin is approved for use at doses up to 2550 mg/day, with an individual dose of immediate-release metformin typically not exceeding 1000 mg. See Glucophage (metformin hydrochloride) [package insert], Princeton, NJ, Bristol-Myers Squibb Company, May 2018. As such, a dose of 1000 mg of immediate-release metformin was used in the current study to maximize the potential of detecting an interaction.

The safety and tolerability of these proposed single doses were considered acceptable for administration to the healthy subjects participating in this study given the stated inclusion/exclusion criteria and the specified safety monitoring.

### Selection and Timing of Dose for Each Subject

Each subject received each treatment once during the study. All study drug was administered at 8:00 AM (+2 hours). For Treatment B, the dose of Compound 1 was administered 2 hours (+2 minutes) prior to the dose of metformin to allow sufficient time for tissue distribution of Compound 1 in order to maximize the potential of detecting an interaction. Subjects were required to fast (defined as no food or drink with the exception of water) for a minimum of 10 hours prior to administration of metformin in each treatment period and then continued fasting for a minimum of 4 hours after each administration of metformin. Water was permitted ad libitum up to 1 hour before and starting 1 hour after administration of Compound 1 and/or metformin. Each dose was administered with approximately 240 mL of water.

### Blinding

This was an open-label study and blinding procedures were not required.

### Prior and Concomitant Therapy

Excluded and restricted medications and/or procedures

Subjects were not permitted to take the following medications within 14 days prior to the first dose of study drug or 5 half-lives, whichever was longer, until after discharge from the study site:
Any prescription medications, including topicals;
Over-the-counter medications (other than occasional use of acetaminophen or nonsteroidal anti-inflammatory drugs, such as ibuprofen or naproxen, according to the package insert);
Herbal supplements;
Dietary supplements; or
Nutraceuticals.

Use of over-the-counter topical medications may have been permitted in consultation with the Sponsor. In addition, use of medications (other than those listed above) for which 5 half-lives exceeded 14 days must have been discussed with and approved by the Sponsor prior to subject enrollment.

Subjects were not permitted to use corticosteroids (systemic or extensive topical) within 3 months (90 days) prior to the first dose of study drug.

Subjects who received a radiologic scan with contrast within 14 days prior to the first dose of study drug were excluded. Subjects who completed surgical procedures within 4 weeks of Check-In (other than minor cosmetic surgery or minor dental procedures) or subjects with planned elective surgeries during the treatment period were excluded.

Subjects who were currently undergoing treatment with weight loss medication or who had received prior weight loss surgery (eg, gastric bypass surgery) were excluded.

Subjects who were actively participating in an experimental therapy study, who received experimental therapy with a small molecule other than Compound 1 within 30 days of the first dose of study drug or 5 half-lives, whichever was longer, or received experimental therapy with a large molecule within 90 days of the first dose of study drug or 5 half-lives, whichever was longer, were excluded.

### Contraception

Subjects of reproductive potential were required to use 2 medically accepted highly effective forms of birth control.

Medically accepted, highly effective methods of birth control for male subjects with female partners of childbearing potential must have been used from Day 1 through 90 days after administration of the final dose of study drug and included the following:
Latex condom with spermicide;
Partner's use of diaphragm with intravaginal spermicide;
Partner's use of cervical cap with spermicide;
Partner's use of indwelling intrauterine device (hormonal or nonhormonal);
Partner's use of implanted contraceptives; or
Partner's use of oral contraceptives.

Medically accepted, highly effective methods of birth control for female subjects with male partners must have been used from Day −14 until 60 days following administration of the final dose of study drug and included the following:
Diaphragm with intravaginal spermicide;
Cervical cap with spermicide;
Nonhormonal indwelling intrauterine device (for at least 12 weeks before the Screening Visit); or
Partner's use of latex condom with spermicide.

### Dietary and Lifestyle Considerations

Subjects were required to fast (defined as no food or drink with the exception of water) for a minimum of 10 hours prior to administration of metformin in each treatment period and then continued fasting for a minimum of 4 hours after each administration of metformin. Water was permitted ad libitum up to 1 hour before and starting 1 hour after administration of Compound 1 and/or metformin.

Subjects must have abstained from alcohol; caffeine and/or xanthine-containing products (ie, coffee, tea, chocolate, and caffeine-containing sodas, colas, energy drinks, etc); grapefruit and grapefruit products; star fruit and star fruit products; Seville oranges; marmalade; cranberry juice; garlic; charcoal grilled/barbecued meat; broccoli, Brussels sprouts; St. John's wort or any food substance that can inhibit or induce CYP or drug transporters, or affect coagulation; and vitamin waters from 1 week prior to administration of the first dose of study drug through discharge from Treatment Period 2.

Subjects must have refrained from contact sports and strenuous exercise from 5 days prior to first dose of study drug through discharge from Treatment Period 2.

Documentation of prior and concomitant medication use

All medications or supplements taken from 28 days prior to the first dose of the study drug through the follow-up phone call were recorded in the subject's chart and the corresponding eCRF.

### Appropriateness of Measurements

The PK and safety measurements in this study are widely used and recognized as reliable, accurate, and relevant.

### Pharmacokinetic Endpoints

The following plasma PK parameters were determined for Compound 1 and its primary metabolite (Compound 1-metabolite):
Cₘₐₓ, determined directly from the concentration-time profile;
Tₘₐₓ, defined as the first time point with the maximum value, if the maximum value occurred at more than 1 time point;
Apparent first-order terminal elimination rate constant calculated from a semi-logarithmic plot of the plasma concentration versus time curve (λ_{z}), calculated by linear least squares (LS) regression analysis using points in the terminal logarithmic-linear phase;
AUC from time 0 to 24 hours (AUC₀₋₂₄);
AUC from time 0 to 72 hours;
AUC from time 0 to infinity (AUC_{0-inf}) (Compound 1 only), calculated as AUC from time 0 to the time of the last quantifiable plasma concentration (AUC₀₋ₜ)+last quantifiable plasma concentration (Cₗₐₛₜ)/λ_{z}; and
Percent of AUC_{0-inf} extrapolated (AUC_{% extrap}) (Compound 1 only), represented as (1−AUC₀₋ₜ/AUC_{0-inf})×100.

The following plasma PK parameters were determined for metformin:
Cₘₐₓ, determined directly from the concentration-time profile;
Tₘₐₓ, defined as the first time point with the maximum value, if the maximum value occurred at more than 1 time point;
λ_{z}, calculated by linear LS regression analysis using points in the terminal logarithmic-linear phase;
AUC₀₋₂₄;
AUC₀₋ₜ;
AUC_{0-inf}, calculated as (AUC₀₋ₜ+Cₗₐₛₜ/λ_{z});
AUC_{% extrap}, represented as (1−AUC₀₋ₜ/AUC_{0-inf})×100; and
t_{1/2}, calculated as ln(2)/λ_{z}.

The following urine PK parameters were determined for metformin:
Cumulative amount of metformin excreted in the urine (cumulative amount of drug excreted in the urine [Ae]);
Renal clearance, calculated as Ae/AUC; and
Fraction of the dose excreted renally (Fe), calculated as 100×Ae/Dose.

### Safety Endpoints

The following safety assessments were performed:
AEs and SAEs;
Clinical laboratory test assessments;
Vital signs including heart rate, BP (including orthostatic BP when indicated), respiration rate, and temperature;
12-lead ECGs;
Physical examinations; and
Height, weight, and BMI.

### Demographic and Baseline Characteristics

The following demographic and baseline characteristics were listed and summarized by treatment sequence and overall with descriptive statistics or counts and percentages of subjects for the Safety Population and was repeated for all other analysis populations if they were different from the Safety Population:
Age (years);
Sex;
Childbearing potential; F
Ethnicity (Hispanic or Latino, Not Hispanic or Latino, Not reported, Unknown);
Race (Asian, American Indian or Alaska Native, Black or African American, Native Hawaiian or Other Pacific Islander, White, Other);
Height (cm);
Weight (kg); and
BMI (kg/m²).

### Medical and Surgical History

Medical/surgical history was collected at Screening. Medical history was re-evaluated at Check-In for Treatment Period 1 to confirm eligibility, and any new signs/symptoms were reported as updated medical history. Medical history reported terms were coded to SOC and PT using MedDRA (Version 23.1). Medical history by SOC and PT was summarized by treatment sequence and in total for the Safety Population as well as listed.

### Prior and Concomitant Medications

Prior and concomitant medications were coded using the WHO Drug Dictionary (Version September 2020G B3). All medications or supplements taken from 28 days prior to the first dose of the study drug through the follow-up phone call were recorded.

Prior medications were defined as those medications (both prescribed and over-the-counter) taken prior to the first dose of study drug. Medications taken at and after the time of first dose during the study were defined as concomitant medications.

All prior and concomitant medications were listed and summarized by ATC classification, PT, and treatment group for the Safety Population.

### Study Drug Exposure and Compliance

Study drug administration data were listed by treatment group for all subjects in the Safety Population.

### Pharmacokinetic Analyses

### Pharmacokinetic Sample Collection

Blood and urine samples were collected to evaluate PK. All PK time points were relative to the time of metformin administration. However, it should be noted that for Compound 1, the total duration for PK sampling post metformin dosing was 2 hours longer than for the corresponding metformin PK sampling. The actual date and time of collection of each PK sample were recorded and utilized to calculate the actual sampling intervals relative to the relevant study drug (Compound 1 or metformin), which were utilized in the PK calculations.

Samples that were collected immediately prior to administration of Compound 1 or metformin, as well as the −0.5 hour sample may have been collected up to 10 minutes early. The following windows were permitted for the collection of the other PK blood samples: ±1 minute for samples collected between −1.5 hours and −1 hours, inclusive, as well as samples from 0.5 hours through 6 hours post metformin administration; ±2 minutes for samples collected >6 and ≤16 hours post metformin administration; and ±5 minutes for samples collected >16 hours post metformin administration.

Actual sampling times that were outside the sampling time windows underwent a case-by-case review and all were included in the individual and mean time course profiles and parameters.

Handling missing or below the lower limit of quantification data

For PK concentration data, if the actual sampling time was missing but a valid concentration value had been measured, the concentration value was flagged, and the scheduled time point was used for the calculation of PK parameters.

In cases of missing predose values for each period, the missing components were assumed as 0. For the other cases, the missing data were not imputed.

For the individual concentration and PK parameter calculation of each treatment group, the following rules were applied:
If 1 or more below the lower limit of quantification (BLQ) values occurred before the first measurable concentration, they were assigned a value of 0; and
If BLQ values occurred between measurable concentrations in a profile or after the last measurable concentration, the BLQ was handled in the following manner:
   For all truncated AUCs or AUC from AUC₀₋ₜ calculations, the BLQ was assigned a value of 0; and
For terminal phase λ_{z} and associated parameter calculations, the BLQ was omitted (set to missing).

For the concentration summary and mean concentration plot preparation of each period, the following rules were applied:
Mean concentration at any individual time point was only calculated if at least half of the subjects had valid values (ie, quantifiable and not missing) at this time point for each treatment group;
In cases where a mean value was not calculated due to the above criterion not being met, the value was set to missing; and
BLQ values were set to 0.

### Pharmacokinetic Concentration Data

Plasma concentrations of Compound 1, its primary metabolite (Compound 1-metabolite), and metformin were listed by individual subject and summarized by treatment using descriptive statistics for the PK Evaluable Population. Compound 1, Compound 1-metabolite, and metformin plasma concentrations were plotted against time points by treatment (mean and individual). Mean concentrations were plotted against nominal sampling times, while individual concentrations were plotted against actual sampling times.

Urine concentrations of metformin were listed by individual subject and summarized by treatment using descriptive statistics for the PK Evaluable Population. Plots of Ae by time point and treatment (individual and mean) were also presented.

### Pharmacokinetic Parameters

Plasma and urine PK parameters were determined using non-compartmental methods as appropriate. For a list of PK parameters, see Section 0.

Actual collection times were used in PK parameter calculations. The Linear-Logarithmic Trapezoidal method (equivalent to the Linear Up/Logarithmic Down option in WinNonlin) were used in the computation of all AUC values. In order to estimate the apparent first-order terminal elimination constant, λz, linear regression of concentration on a logarithmic scale versus time were performed using at least 3 data points. Uniform weighting was selected to perform the regression analysis to estimate λ_{z}. The constant λ_{z} was assigned but flagged if 1 of the following occurred:
1. The terminal elimination phase was not linear (as it appears on a semi-logarithmic scale);
2. The terminal elimination rate constant indicated a positive slope (λ_{z}>0);
3. Tₘₐₓ was one of the 3 last data points;
4. The adjusted regression coefficient was less than 0.8; or
5. The AUC_{% extrap} exceeded 20%.

These λz values and λz-derived parameters were listed but excluded from statistical analysis.

No value for λz, AUC0-inf, AUC % extrap, or t½ were reported for cases that did not exhibit an acceptable terminal logarithmic-linear phase in the concentration-time profile.

No PK parameters were calculated for subjects with 2 or fewer detectable concentrations in their PK profile.

Parameters were listed by individual subject and summarized by treatment using descriptive statistics for the PK Population.

No drug-drug interaction effect boundary evaluation

Logarithmic transformations of PK parameters of metformin were analyzed using a mixed model including terms for sequence, treatment group, and period as fixed effects, and subject nested within sequence as a random effect. Geometric mean ratios and associated 90% confidence intervals (CIs) were presented for Cₘₐₓ, AUC_{0-inf}, and AUC₀₋ₜ values following administration of metformin alone and metformin coadministered with Compound 1. If the 90% CIs on the geometric mean ratio were within 80% to 125% for Cₘₐₓ and AUC, the absence of a drug-drug interaction was concluded.

The PK parameters analyses were based on the PK Population.

### Adverse Events

An AE was defined as any untoward medical occurrence in a clinical study subject administered a pharmaceutical product, which did not necessarily have a causal relationship with this treatment. An AE could therefore have been any unfavorable and/or unintended sign (including an abnormal laboratory finding), symptom, or disease temporally associated with the use of the study drug, whether or not related to the study drug.

AEs were coded using the MedDRA (Version 23.1).

The Investigator assessed the severity (intensity) of each AE as mild, moderate, or severe.

The relationship of an AE to the administration of the study drug was assessed as: no (unrelated, not related, or unlikely to be related) or yes (possibly, probably, or definitely related).

Treatment-emergent adverse events

A TEAE was defined as an AE that emerged, having been absent prior to the study, or an AE that worsened in severity after the first dose of any drug in this study. TEAEs were summarized by MedDRA SOC and PT for each treatment and overall. The number and percentage of subjects experiencing TEAEs and the number of TEAEs were tabulated. Subjects reporting more than 1 AE for a given MedDRA PT were counted only once for that term using the most severe incident. Subjects reporting more than 1 type of event within a SOC were counted only once for that SOC. The Safety Population was used for all the safety analyses. The following summaries of AEs were presented for each part:
Overall summary of TEAEs by treatment and overall with a breakdown by AE maximum severity;
TEAEs by SOC and PT by treatment;
TEAEs by SOC, PT, and relationship to study drug or metformin by treatment;
TEAEs by SOC, PT, and maximum severity by treatment; and
TEAEs by SOC, PT, maximum severity, and the relationship to the study drug by treatment.

Likewise, SAEs were summarized similarly, if possible.

Separate listings were prepared for SAEs, AEs leading to death, and AEs leading to study discontinuation.

### Changes

Changes made are summarized below:
It was clarified that subjects receiving 1 or more doses of Compound 1 or metformin were not intended to be replaced;
It was clarified that Day −2 procedures (SARS-CoV-2 testing) were required the day prior to check in for both treatment periods;
A ±2-minute window for the administration of Compound 1 in Treatment B was added;
The following additions and clarifications for the collection of the PK samples were made:
   Additional samples were collected at −2 hours, −1.5 hours, −1 hour, 0.5 hour, 14 hours, and 22 hours;
   It was noted that all PK time points were relative to the time of metformin administration; and
   A window was added to permit the PK samples immediately prior to administration of Compound 1 and/or metformin as well as the −0.5 hour sample to be collected up to 10 minutes early.
Vital signs (including orthostatic vital signs) were added prior to administration of metformin on Day 1 and prior to collection of the 24-hour PK collection on the morning of Day 2 for Treatment A, and prior to administration of Compound 1 on Day 1 and prior to collection of the 22-hour PK collection on Day 2 for Treatment B;
An ECG was added prior to Compound 1 administration for Treatment B; and
The Schedule of Procedures was revised and updated to reflect updates made to the Clinical Study Protocol and to provide additional clarification.

### Study Subjects

### Disposition of Subjects

Table 20 summarizes subject disposition by treatment sequence for the Safety Population.

A total of 27 subjects were randomized to 1 of 2 treatment sequences: AB (14 subjects) or BA (13 subjects). Of the randomized subjects, 26 subjects completed both treatment periods.

One subject withdrew consent as a result of a family emergency.

TABLE 20
Subject Disposition by Treatment Sequence-Safety Population

| | Sequence AB | Sequence BA | Total |
|---|---|---|---|
| | (N = 14) | (N = 13) | (N = 27) |
| Category | n (%) | n (%) | n (%) |
| Randomized | 14 (100.0) | 13 (100.0) | 27 (100.0) |
| Dosed | 14 (100.0) | 13 (100.0) | 27 (100.0) |
| Completed Treatment Period 1 | 14 (100.0) | 13 (100.0) | 27 (100.0) |
| Completed Treatment Period 2 | 14 (100.0) | 12 (92.3) | 26 (96.3) |
| Completed the study | 14 (100.0) | 12 (92.3) | 26 (96.3) |
| Prematurely withdrew from the study | 0 (0.0) | 1 (7.7) | 1 (3.7) |
| Primary reasons for early withdrawal | | | |
| Withdrawal of consent | 0 (0.0) | 1 (7.7) | 1 (3.7) |
| Prematurely withdrew the study due to | 0 (0.0) | 0 (0.0) | 0 (0.0) |
| COVID-19 pandemic | | | |

Note 1:
Treatment A: a single 1000 mg dose of immediate-release metformin, Treatment B: a single 1000 mg dose of immediate-release metformin coadministered with a 10 mg dose of Compound 1.
Note 2:
Percentages were calculated using the number of subjects in the Safety Population in each treatment sequence as the denominator.
Note 3:
A subject was considered randomized the moment they were dosed with the study drug. If a subject was given a randomization number but withdrew prior to dosing, the subject was considered not randomized and their randomization number was used for the next available subject.
Note 4:
The Safety Population consisted of all randomized subjects who received any study drug (Compound 1 or metformin).
COVID-19 = Coronavirus Disease 2019.

### Demographic and Other Baseline Characteristics

Table 21 summarizes demographic and baseline characteristics for the Safety Population.

The mean age of subjects was approximately 37 years and the mean BMI was approximately 26 kg/m². Approximately half of the subjects were White, and half were Black or African American. The majority of subjects were male and not Hispanic or Latino. Subjects in Sequence BA were, on average, slightly older (mean age of 41 years) and had a more pronounced prevalence of males than those in Sequence AB (mean age of 33 years); however, these observed differences are considered unlikely to influence the data interpretation and conclusions.

TABLE 21
Demographic and Baseline Characteristics-Safety Population

| | | | |
|---|---|---|---|
| Characteristic | Sequence AB | Sequence BA | Total |
| Statistics/Category | (N = 14) | (N = 13) | (N = 27) |

Age (years)

| | | | |
|---|---|---|---|
| n | 14 | 13 | 27 |
| Mean (SD) | 33.0 (6.21) | 40.7 (9.49) | 36.7 (8.73) |
| Sex, n (%) | | | |
| Male | 8 (57.1) | 11 (84.6) | 19 (70.4) |
| Female | 6 (42.9) | 2 (15.4) | 8 (29.6) |

Childbearing potential, n (%) [1]

| | | | |
|---|---|---|---|
| Yes | 6 (100.0) | 2 (100.0) | 8 (100.0) |
| Race, n (%) | | | |
| White | 6 (42.9) | 9 (69.2) | 15 (55.6) |
| Black or African | 8 (57.1) | 4 (30.8) | 12 (44.4) |
| American | | | |

Ethnicity, n (%)

| | | | |
|---|---|---|---|
| Hispanic or Latino | 1 (7.1) | 3 (23.1) | 4 (14.8) |
| Not Hispanic or Latino | 13 (92.9) | 10 (76.9) | 23 (85.2) |

Height (cm)

| | | | |
|---|---|---|---|
| n | 14 | 13 | 27 |
| Mean (SD) | 170.29 (7.511) | 174.67 (10.253) | 172.40 (9.040) |

Weight (kg)

| | | | |
|---|---|---|---|
| n | 14 | 13 | 27 |
| Mean (SD) | 73.83 (9.979) | 78.75 (10.215) | 76.20 (10.209) |

Body mass index (kg/m²)

| | | | |
|---|---|---|---|
| n | 14 | 13 | 27 |
| Mean (SD) | 25.38 (2.495) | 25.82 (2.560) | 25.59 (2.487) |

Note 1:
% = 100 × n/N.
Note 2:
Treatment A: a single 1000 mg dose of immediate-release metformin, Treatment B: a single 1000 mg dose of immediate-release metformin coadministered with a 10 mg dose of Compound 1.
Note 3:
Baseline was defined as readings or measurements obtained prior to dosing at Day 1 of Treatment Period 1. If the reading at Day 1 predose was missing, the last reading before first dosing was set as baseline.
[1] Percentages were calculated based on female count.
SD = standard deviation.

### Pharmacokinetic Results

### Pharmacokinetic Analyses

For the bioanalytical reports of Compound 1 and metformin in human plasma samples and metformin in human urine samples, see Appendix 16.1.13.

### Plasma Pharmacokinetics of Metformin in the Presence and Absence of Compound 1

Error! Reference source not found.5 displays the plots of mean (+SD) plasma metformin concentrations over 24 hours postdose by treatment on linear and semi-logarithmic scales for the PK Population.

The plasma concentration-time course curves for metformin were nearly identical in the presence and absence of Compound 1.

Table 22 summarizes the plasma PK parameters for metformin for the PK Evaluable Population.

The plasma PK parameters of metformin were consistent with what would be expected based on the package insert for immediate-release metformin. See Glucophage (metformin hydrochloride) [package insert]. Princeton, NJ. Bristol-Myers Squibb Company. May 2018.

TABLE 22
Summary of Plasma PK Parameters for Metformin-PK Evaluable
Population

| PK Parameter | Statistic | Treatment A | Treatment B |
|---|---|---|---|
| Cₘₐₓ (ng/mL) | n | 26 | 27 |
| | Mean (SD) | 1765.4 (344.4) | 1793.1 (503.9) |
| Tₘₐₓ (h) | n | 26 | 27 |
| | Median (min, max) | 2.0 (1.0, 4.0) | 2.0 (1.0, 3.5) |
| λ_{z} (1/h) | n | 25 | 26 |
| | Mean (SD) | 0.052 (0.016) | 0.043 (0.020) |
| t½ (h) | n | 26 | 27 |
| | Mean (SD) | 5.5 (1.0) | 6.2 (1.4) |
| AUC₀₋₂₄ | n | 26 | 27 |
| (h*ng/ml) | Mean (SD) | 10552.3 (1770.0) | 10388.9 (2912.8) |
| AUC₀₋ₜ | n | 26 | 27 |
| (h*ng/ml) | Mean (SD) | 11158.8 (1810.5) | 11043.6 (2898.8) |
| AUC_{0-inf} | n | 25 | 26 |
| (h*ng/ml) | Mean (SD) | 11224.8 (1849.8) | 11319.2 (2905.1) |
| AUC_{%extrap} (%) | n | 25 | 26 |
| | Mean (SD) | 0.9 (1.5) | 2.6 (4.2) |

Note 1:
Treatment A: a single 1000 mg dose of immediate-release metformin, Treatment B: a single 1000 mg dose of
immediate-release metformin coadministered with a 10 mg dose of (R)-Compound 1.
Note 2:
Metformin t½ was calculated using values up to the nominal 24-hour postdose time point only. All other metformin parameters were calculated using the full 0 to 72 hour profile.
λ_{z} = apparent first-order terminal elimination rate constant calculated from a semi-logarithmic plot of the plasma concentration versus time curve;
AUC_{%extrap} = percent of area under the concentration-time curve from time 0 to infinityextrapolated;
AUC₀₋₂₄ = area under the concentration-time curve from time 0 to 24 hours;
AUC_{0-inf} = area under the concentration-time curve from time 0 to infinity;
AUC₀₋ₜ = area under the concentration-time curve from time 0 to the time of the last quantifiable plasma concentration;
Cₘₐₓ = maximum observed plasma concentration;
max = maximum;
min = minimum;
PK = pharmacokinetic(s);
SD = standard deviation;
t½ = terminal phase elimination half-life;
Tₘₐₓ = time to maximum observed plasma concentration.

Table 23 summarizes the analysis of the plasma PK parameters for metformin for the PK Evaluable Population.

The ratios of geometric LS mean for Treatment B:Treatment A for Cₘₐₓ, AUC_{0-inf}, and AUC₀₋ₜ all approximated 100%, with 90% CI values falling within the acceptance range of 80% to 125%, indicating that systemic exposure to metformin was not affected by Compound 1.

TABLE 23
Analysis of Plasma PK Parameters for
Metformin - PK Evaluable Population

| | | | | | Ratio of | |
|---|---|---|---|---|---|---|
| | Treatment A | | Treatment B | | Geom LS | |
| | | Geom | | Geom | Mean | 90% CI for |
| PK | | LS | | LS | (B/A) | Ratio [2] |
| Parameter | n | Mean [1] | n | Mean [1] | (%) | (%) |
| Cₘₐₓ | 26 | 1732.80 | 26 | 1712.68 | 98.84 | (91.33, 106.96) |
| (ng/ml) | | | | | | |
| AUC_{0-inf} | 24 | 11084.83 | 24 | 11103.06 | 100.16 | (94.41, 106.27) |
| (h*ng/ml) | | | | | | |
| AUC₀₋ₜ | 26 | 11040.29 | 26 | 10683.45 | 96.77 | (90.72, 103.21) |
| (h*ng/mL) | | | | | | |

Note 1:
Treatment A: a single 1000 mg dose of immediate-release metformin (reference treatment), Treatment B: a single 1000 mg dose of immediate-release metformin coadministered with a 10 mg dose of Compound 1 (test treatment).
Note 2:
Logarithmic transformations of PK parameters of metformin were analyzed using a mixed model including terms for sequence, treatment group, and period as fixed effects, and subject nested within sequence as a random effect. A subject must have had a calculable PK parameter in both treatments in order to be included in the analysis for that parameter.
[1] Geom LS means are the LS means from the mixed model presented after back transformation to the original scale.
[2] The 90% CI were presented after back transformation to the original scale.
AUC_{0-inf} = area under the concentration-time curve from time 0 to infinity; AUC₀₋ₜ = area under the concentration-time curve from time 0 to the time of last quantifiable plasma concentration; CI = confidence interval; Cₘₐₓ = maximum observed plasma concentration; Geom = geometric; LS = least squares; PK = pharmacokinetic(s).

### Urine Pharmacokinetics of Metformin in the Presence and Absence of Compound 1

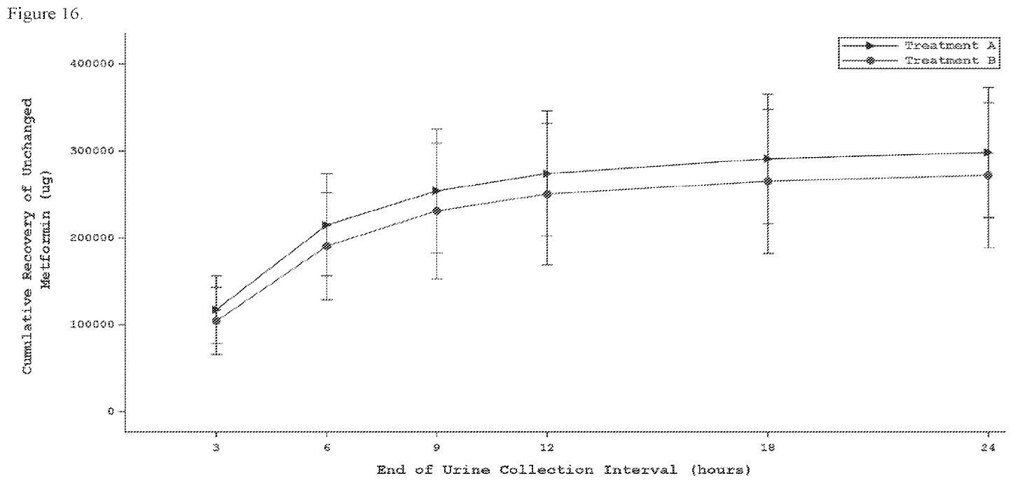

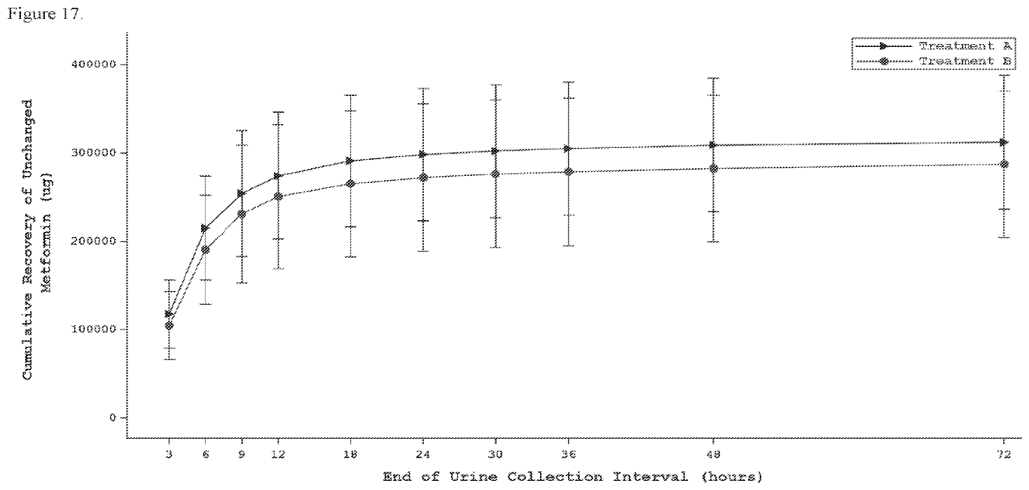

FIG. 16 and FIG. 17 display the plots of mean (+SD) Ae of metformin by treatment over 24 and 72 hours postdose, respectively, on a linear scale for the PK Population.

Consistent with what would be expected based on the package insert for immediate-release metformin, approximately 30% of the metformin dose was excreted in the urine and the majority of excretion occurred during the first 24 hours postdose. See Glucophage (metformin hydrochloride) [package insert]. Princeton, NJ. Bristol-Myers Squibb Company. May 2018. Plots of cumulative urinary excretion of metformin were qualitatively and quantitatively similar in the presence and absence of Compound 1. Despite some variability within and across individuals, there were no consistent trends on an individual basis to suggest that urine excretion of metformin was reduced by Compound 1.

Table 24 summarizes the Fe for metformin for the PR Evaluable Population.

Administration of Compound 1 2 hours prior to metformin dosing did not result in a reduction in metformin renal clearance.

TABLE 24
Summary of Urine PK Parameters for Metformin-PK Evaluable Population

| PK Parameter | Statistic | Treatment A | Treatment B |
|---|---|---|---|
| Fe₀₋₃ (%) | n | 26 | 27 |
| | Mean (CV %) | 11.76 (33.0) | 10.47 (36.6) |
| Fe₀₋₆ (%) | n | 26 | 27 |
| | Mean (CV %) | 21.50 (27.2) | 19.06 (32.4) |
| Fe₀₋₉ (%) | n | 26 | 27 |
| | Mean (CV %) | 25.40 (28.1) | 23.11 (33.8) |
| Fe₀₋₁₂ (%) | n | 26 | 27 |
| | Mean (CV %) | 27.42 (26.2) | 25.07 (32.5) |
| Fe₀₋₁₈ (%) | n | 26 | 27 |
| | Mean (CV %) | 29.11 (25.6) | 26.52 (31.3) |
| Fe₀₋₂₄ (%) | n | 26 | 27 |
| | Mean (CV %) | 29.82 (25.1) | 27.21 (30.6) |
| Fe₀₋₃₀ (%) | n | 26 | 27 |
| | Mean (CV %) | 30.21 (24.9) | 27.62 (30.3) |
| Fe₀₋₃₆ (%) | n | 26 | 27 |
| | Mean (CV %) | 30.50 (24.7) | 27.87 (30.0) |
| Fe₀₋₄₈ (%) | n | 26 | 27 |
| | Mean (CV %) | 30.90 (24.5) | 28.25 (29.4) |
| Fe₀₋₇₂ (%) | n | 26 | 27 |
| | Mean (CV %) | 31.23 (24.2) | 28.72 (28.9) |

Note 1:
Treatment A: a single 1000 mg dose of immediate-release metformin,
Treatment B: a single 1000 mg dose of immediate-release metformin coadministered with a 10 mg dose of Compound 1.
CV = coefficient of variation;
Fe = fraction of the dose excreted renally;
Fe₀₋₃ = cumulative fraction excreted from 0 to 3 hours;
Fe₀₋₆ = cumulative fraction excreted from 0 to 6 hours;
Fe₀₋₉ = cumulative fraction excreted from 0 to 9 hours;
Fe₀₋₁₂ = cumulative fraction excreted from 0 to 12 hours;
Fe₀₋₁₈ = cumulative fraction excreted from 0 to 18 hours;
Fe₀₋₂₄ = cumulative fraction excreted from 0 to 24 hours;
Fe₀₋₃₀ = cumulative fraction excreted from 0 to 30 hours;
Fe₀₋₃₆ = cumulative fraction excreted from 0 to 36 hours;
Fe₀₋₄₈ = cumulative fraction excreted from 0 to 48 hours;
Fe₀₋₇₂ = cumulative fraction excreted from 0 to 72 hours;
PK = pharmacokinetic(s).

### Plasma Pharmacokinetics of Compound 1

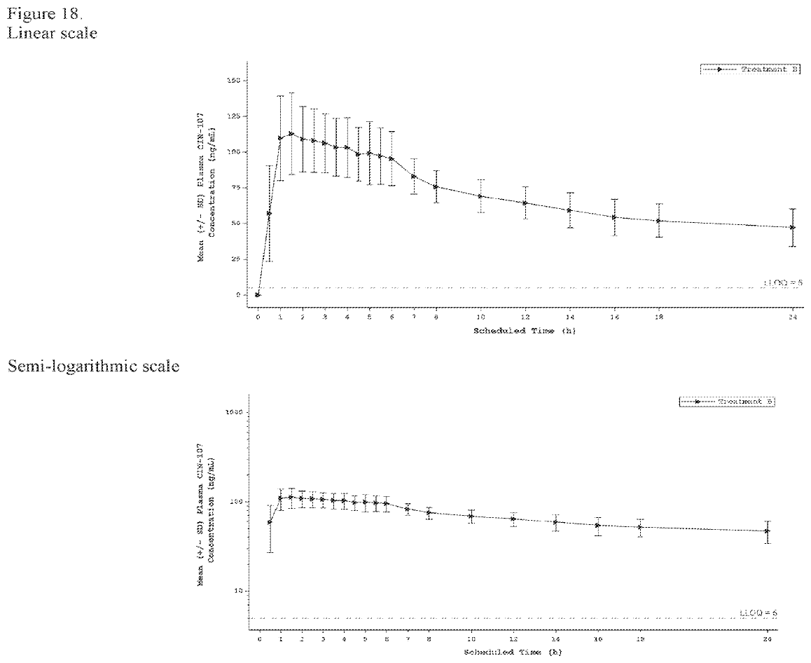

FIG. 18 displays the plots of mean (±SD) plasma Compound 1 concentrations over 24 hours postdose by treatment on linear and semi-logarithmic scales for the PR Population.

Table 25 summarizes the plasma PR parameters for Compound 1 for the PR Evaluable Population.

The plasma PR parameters of Compound 1 were consistent with those observed in previous studies.

TABLE 25
Summary of Plasma PK Parameters for Compound
1-PK Evaluable Population

| PK Parameter | Statistic | Treatment B |
|---|---|---|
| Cₘₐₓ (ng/mL) | n | 27 |
| | Mean (SD) | 118.9 (25.6) |
| Tₘₐₓ (h) | n | 27 |
| | Median (min, max) | 1.3 (1.0, 4.0) |
| λ_{z} (1/h) | n | 20 |
| | Mean (SD) | 0.033 (0.008) |
| AUC₀₋₂₄ (h*ng/mL) | n | 27 |
| | Mean (SD) | 1663.0 (303.4) |
| AUC₀₋₇₂ (h*ng/ml) | n | 27 |
| | Mean (SD) | 2872.7 (707.0) |
| AUC_{0-inf} (h*ng/mL) | n | 20 |
| | Mean (SD) | 3025.0 (811.3) |
| AUC_{%extrap} (%) | n | 20 |
| | Mean (SD) | 9.9 (5.2) |

Note 1:
Treatment B: a single 1000 mg dose of immediate-release metformin coadministered with a 10 mg dose of Compound 1.
λ_{z} = apparent first-order terminal elimination rate constant calculated from a semi-logarithmic plot of the plasma concentration versus time curve;
AUC_{%extrap} = percent of area under the concentration-time curve from time 0 to infinity extrapolated;
AUC₀₋₂₄ = area under the concentration-time curve from time 0 to 24 hours;
AUC₀₋₇₂ = area under the concentration-time curve from time 0 to 72 hours;
AUC_{0-inf} = area under the concentration-time curve from time 0 to infinity;
Cₘₐₓ = maximum observed plasma concentration;
max = maximum;
min = minimum;
PK = pharmacokinetic(s);
SD = standard deviation;
Tₘₐₓ = time to maximum observed plasma concentration.

### Pharmacokinetic Conclusions

The ratios of geometric LS mean for Treatment B:Treatment A for metformin plasma Cₘₐₓ, AUC0-inf, and AUC0-t all approximated 100%, with 90% CI values falling within the acceptance range of 80% to 125%, indicating that systemic exposure to metformin was not affected by Compound 1.

Consistent with the findings in plasma, plots of cumulative urinary excretion of metformin were qualitatively and quantitatively similar in the presence and absence of Compound 1, indicating that Compound 1 did not result in a reduction in metformin renal clearance.

### Adverse Events

### Brief Summary of Adverse Events

There were no deaths, SAEs, or discontinuations due to a TEAE. Overall, 7 subjects experienced a total of 15 TEAEs: 5 (19.2%) subjects experienced a total of 6 TEAEs following administration of metformin alone (Treatment A), and 6 (22.2%) subjects experienced a total of 9 TEAEs following coadministration of metformin and Compound 1 (Treatment B). Four subjects experienced a TEAE following both treatments. All TEAEs experienced by subjects were mild in severity, and no subjects experienced moderate or severe TEAEs. See table 26.

TABLE 26
Overview of TEAEs-Safety Population

| | Treatment A | Treatment B | Total |
|---|---|---|---|
| | (N = 26) | (N = 27) | (N = 27) |
| | n (%) | n (%) | n (%) |
| | e | e | e |
| | 5 (19.2) | 6 (22.2) | 7 (25.9) |
| Any TEAEs | 6 | 9 | 15 |

Any TEAEs by maximum severity

| | | | |
|---|---|---|---|
| Mild | 5 (19.2) | 6 (22.2) | 7 (25.9) |
| | 6 | 9 | 15 |
| Moderate | 0 (0.0) | 0 (0.0) | 0 (0.0) |
| | 0 | 0 | 0 |
| Severe | 0 (0.0) | 0 (0.0) | 0 (0.0) |
| | 0 | 0 | 0 |
| Any Compound 1-related TEAEs | 0 (0.0) | 0 (0.0) | 0 (0.0) |
| | 0 | 0 | 0 |
| | 4 (15.4) | 6 (22.2) | 6 (22.2) |
| Any metformin-related TEAEs | 5 | 7 | 12 |

Any Compound 1-related TEAEs by maximum severity

| | | | |
|---|---|---|---|
| Mild | 0 (0.0) | 0 (0.0) | 0 (0.0) |
| | 0 | 0 | 0 |
| Moderate | 0 (0.0) | 0 (0.0) | 0 (0.0) |
| | 0 | 0 | 0 |
| | 0 (0.0) | 0 (0.0) | 0 (0.0) |
| Severe | 0 | 0 | 0 |

Any metformin-related TEAEs by maximum severity

| | | | |
|---|---|---|---|
| Mild | 4 (15.4) | 6 (22.2) | 6 (22.2) |
| | 5 | 7 | 12 |
| Moderate | 0 (0.0) | 0 (0.0) | 0 (0.0) |
| | 0 | 0 | 0 |
| Severe | 0 (0.0) | 0 (0.0) | 0 (0.0) |
| | 0 | 0 | 0 |
| Any TEAEs leading to withdrawal of | 0 (0.0) | 0 (0.0) | 0 (0.0) |
| study drug | 0 | 0 | 0 |
| Any Compound 1-related TEAEs | 0 (0.0) | 0 (0.0) | 0 (0.0) |
| leading | | | |
| to withdrawal of study drug | 0 | 0 | 0 |
| Any metformin-related TEAEs leading | 0 (0.0) | 0 (0.0) | 0 (0.0) |
| to withdrawal of study drug | 0 | 0 | 0 |
| | 0 (0.0) | 0 (0.0) | 0 (0.0) |
| Any TEAEs leading to death | 0 | 0 | 0 |

Note 1:
% = 100 × n/N.
Note 2:
The number of events was not presented for summaries by maximum severity.
Note 3:
Treatment A: a single 1000 mg dose of immediate-release metformin,
Treatment B: a single 1000 mg dose of immediate-release metformin coadministered with a 10 mg dose of Compound 1.
A TEAE was defined as an AE that emerged, having been absent prior to the study, or an AE that worsened in severity after the first dose of any drug in this study. Subjects reporting more than 1 AE were counted only once using the most severe incident. AEs were coded using the MedDRA Version 23.1.
AE = adverse event;
e = number of events;
MedDRA = Medical Dictionary for Regulatory Activities;
TEAE = treatment-emergent adverse event.

### Analysis of Adverse Events

All adverse events

Table 27 summarizes TEAEs by SOC and PT for the Safety Population.

The most common SOC of TEAEs was gastrointestinal disorders. A total of 5 (18.5%) subjects experienced diarrhoea: 3 (11.5%) subjects following administration of metformin alone (Treatment A) and 4 (14.8%) subjects following coadministration of metformin and Compound 1 (Treatment B). Two subjects experienced diarrhoea following both treatments. One (3.7%) subject experienced upper abdominal pain following both treatments, 1 (3.7%) subject experienced flatulence following coadministration of metformin and Compound 1 (Treatment B), and 1 (3.7%) subject experienced nausea following administration of metformin alone (Treatment A).

All other TEAEs experienced by subjects were experienced by 1 subject each: dizziness postural, presyncope, vessel puncture site pain, and burns first degree.

TABLE 27
TEAEs by System Organ Class and Preferred Term-Safety Population

| | Treatment A | Treatment B | Total |
|---|---|---|---|
| System Organ Class | (N = 26) | (N = 27) | (N = 27) |
| Preferred Term | n (%) | n (%) | n (%) |
| Subjects with any TEAEs | 5 (19.2) | 6 (22.2) | 7 (25.9) |
| Gastrointestinal disorders | 4 (15.4) | 5 (18.5) | 6 (22.2) |
| Diarrhoea | 3 (11.5) | 4 (14.8) | 5 (18.5) |
| Abdominal pain upper | 1 (3.8) | 1 (3.7) | 1 (3.7) |
| Flatulence | 0 (0.0) | 1 (3.7) | 1 (3.7) |
| Nausea | 1 (3.8) | 0 (0.0) | 1 (3.7) |
| Nervous system disorders | 0 (0.0) | 2 (7.4) | 2 (7.4) |
| Dizziness postural | 0 (0.0) | 1 (3.7) | 1 (3.7) |
| Presyncope | 0 (0.0) | 1 (3.7) | 1 (3.7) |
| General disorders and administration site conditions | 0 (0.0) | 1 (3.7) | 1 (3.7) |
| Vessel puncture site pain | 0 (0.0) | 1 (3.7) | 1 (3.7) |
| Injury, poisoning and procedural complications | 1 (3.8) | 0 (0.0) | 1 (3.7) |
| Burns first degree | 1 (3.8) | 0 (0.0) | 1 (3.7) |

Note 1:
% = 100 × n/N.
Note 2:
Treatment A: a single 1000 mg dose of immediate-release metformin,
Treatment B: a single 1000 mg dose of immediate-release metformin coadministered with a 10 mg dose of Compound 1.
Note 3:
A TEAE was defined as an AE that emerged, having been absent prior to the study, or an AE that worsened in severity after the first dose of any drug in this study.
Subjects reporting more than 1 AE for a given MedDRA preferred term were counted only once for that term using the most severe incident. Subjects reporting more than 1 type of event within a system organ class were counted only once for that system organ class.
AEs were coded using the MedDRA Version 23.1.
AE = adverse event;
MedDRA = Medical Dictionary for Regulatory Activities;
TEAE = treatment-emergent adverse event.
Source: Post-text Table 14.3.1.2.1

Drug-related adverse events

Table 28 summarizes the metformin-related TEAEs by SOC and PT for the Safety Population.

Overall, 6 (22.2%) subjects experienced TEAEs that were considered related to metformin: 4 (15.4%) subjects following administration of metformin alone (Treatment A) and 6 (22.2%) subjects following coadministration of metformin and Compound 1 (Treatment B). Four subjects experienced a metformin-related TEAE following both treatments. A total of 6 (22.2%) subjects experienced gastrointestinal disorders, which was the most common SOC of the metformin-related TEAEs (as one would expect with a single high dose of metformin). Five (18.5%) subjects experienced diarrhoea: 3 (11.5%) subjects following administration of metformin alone (Treatment A) and 4 (14.8%) subjects following coadministration of metformin and Compound 1 (Treatment B). Two subjects experienced diarrhoea following both treatments. One (3.7%) subject experienced abdominal pain upper following both treatments, 1 (3.7%) subject experienced flatulence following coadministration of metformin and Compound 1 (Treatment B), and 1 (3.7%) subject experienced nausea following administration of metformin alone (Treatment A).

One (3.7%) subject also experienced a nervous system disorder of dizziness postural following coadministration of metformin and Compound 1 (Treatment B).

TABLE 28
Metformin-Related TEAEs by System Organ Class and
Preferred Term-Safety Population

| | Treatment A | Treatment B | Total |
|---|---|---|---|
| System Organ Class | (N = 26) | (N = 27) | (N = 27) |
| Preferred Term | n (%) | n (%) | n (%) |
| Subjects with any metformin- | 4 (15.4) | 6 (22.2) | 6 (22.2) |
| related TEAEs | | | |
| Gastrointestinal disorders | 4 (15.4) | 5 (18.5) | 6 (22.2) |
| Diarrhoea | 3 (11.5) | 4 (14.8) | 5 (18.5) |
| Abdominal pain upper | 1 (3.8) | 1 (3.7) | 1 (3.7) |
| Flatulence | 0 (0.0) | 1 (3.7) | 1 (3.7) |
| Nausea | 1 (3.8) | 0 (0.0) | 1 (3.7) |
| Nervous system disorders | 0 (0.0) | 1 (3.7) | 1 (3.7) |
| Dizziness postural | 0 (0.0) | 1 (3.7) | 1 (3.7) |

Note 1:
% = 100 × n/N.
Note 2:
The number of events was not presented for summaries by maximum severity.
Note 3:
Treatment A: a single 1000 mg dose of immediate-release metformin,
Treatment B: a single 1000 mg dose of immediate-release metformin coadministered with a 10 mg dose of Compound 1.
Note 4:
A TEAE was defined as an AE that emerged, having been absent prior to the study, or an AE that worsened in severity after the first dose of any drug in this study.
Subjects reporting more than 1 AE for a given MedDRA preferred term were counted only once for that term using the incident of greatest severity. Subjects reporting more than 1 type of event within a system organ class were counted only once for that system organ class. AEs were coded using the MedDRA Version 23.1.
AE = adverse event;
MedDRA = Medical Dictionary for Regulatory Activities;
TEAE = treatment-emergent adverse event.

No subject experienced any Compound 1-related TEAEs.

### Safety Conclusions

There were no deaths, SAEs, or discontinuations due to a TEAE, and there was no noteworthy increase in the incidence of AEs when metformin and Compound 1 were coadministered versus metformin administered alone. All TEAEs experienced by subjects were mild in severity. The most frequent TEAEs were gastrointestinal-related, as one would expect with a single high dose of metformin. There were no trends or clinically meaningful changes in laboratory parameters or physical examination results. There were no clinically significant changes observed in vital signs or 12-lead ECG findings, including no meaningful changes in QTcF.

The safety results of the current study indicate that metformin was well tolerated when administered alone or 2 hours after a single 10 mg dose of Compound 1. There were no deaths, SAEs, or discontinuations due to a TEAE, and there was no noteworthy increase in the incidence of AEs when metformin and Compound 1 were coadministered versus metformin administered alone. All TEAEs experienced by subjects were mild in severity. The most frequent TEAEs were gastrointestinal-related, as one would expect with a single high dose of metformin. There were no trends or clinically meaningful changes in laboratory parameters or physical examination results. There were no clinically significant changes observed in vital signs or 12-lead ECG findings, including no meaningful changes in QTcF.

### Overall Conclusions

Metformin was well tolerated when administered alone or 2 hours after a dose of Compound 1. Compound 1 did not result in an increase in metformin plasma concentrations or a decrease in metformin renal clearance when compared with administration of metformin alone. Based on the results from this study, dose adjustment of metformin is not considered necessary when metformin is coadministered with Compound 1.

### Aspects

Aspect 1. A method of treating hypertension or primary aldosteronism in a human having a mean seated blood pressure of ≥130/80 mmHg, comprising administering 0.5 to 10 mg/day of (R)-Compound 1 to the human:

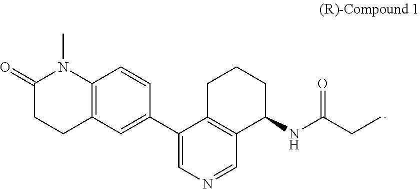 [SMILES: CCC(=O)N[C@@H]1CCCc2c(-c3ccc4c(c3)CCC(=O)N4C)cncc21]

Aspect 2. The method of Aspect 1, wherein the human is on a stable background hypertensive regimen prior to the administration of the (R)-Compound 1.

Aspect 3. The method of Aspect 2, wherein the stable background hypertensive regimen comprising ≥3 antihypertensive agents.

Aspect 4. The method of Aspect 3, wherein one of the antihypertensive agents is a diuretic.

Aspect 5. The method of any one of the preceding Aspects, wherein the administration of the (R)-Compound 1 results in a mean decrease from baseline in seated systolic blood pressure (SBP) after 4 weeks of treatment.

Aspect 6. The method of any one of the preceding Aspects, wherein the administration of the (R)-Compound 1 results in a mean decrease from baseline in seated systolic blood pressure (SBP) after 12 weeks of treatment.

Aspect 7. The method of any one of the preceding Aspects, wherein the administration of the (R)-Compound 1 results in a mean decrease in baseline in seated diastolic blood pressure (DBP) after 4 weeks of treatment.

Aspect 8. The method of any one of the preceding Aspects, wherein the administration of the (R)-Compound 1 results in a mean decrease in baseline in seated diastolic blood pressure (DBP) after 12 weeks of treatment.

Aspect 9. The method of any one of the preceding Aspects, wherein the administration of the (R)-Compound 1 results in a seated blood pressure (BP) of <130/80 mmHg after 4 weeks of treatment.

Aspect 10. The method of any one of the preceding Aspects, wherein the administration of the (R)-Compound 1 results in a seated blood pressure (BP) of <130/80 mmHg after 12 weeks of treatment.

Aspect 11. The method of any one of the preceding Aspects comprising administering about 0.5 mg/day of (R)-Compound 1 to the human.

Aspect 12. The method of any one of Aspects 1 to 10, comprising administering about 1 mg/day of (R)-Compound 1 to the human.

Aspect 13. The method of any one of Aspects 1 to 10, comprising administering about 2 mg/day of (R)-Compound 1 to the human.

Aspect 14. The method of any one of Aspects 1 to 10, comprising administering about 3 mg/day of (R)-Compound 1 to the human.

Aspect 15. The method of any one of Aspects 1 to 10, comprising administering about 4 mg/day of (R)-Compound 1 to the human.

Aspect 16. The method of any one of Aspects 1 to 10, comprising administering about 5 mg/day of (R)-Compound 1 to the human.

Aspect 17. The method of any one of Aspects 1 to 10, comprising administering about 6 mg/day of (R)-Compound 1 to the human.

Aspect 18. The method of any one of Aspects 1 to 10, comprising administering about 7 mg/day of (R)-Compound 1 to the human.

Aspect 19. The method of any one of Aspects 1 to 10, comprising administering about 8 mg/day of (R)-Compound 1 to the human.

Aspect 20. The method of any one of Aspects 1 to 10, comprising administering about 9 mg/day of (R)-Compound 1 to the human.

Aspect 21. The method of any one of Aspects 1 to 10, comprising administering about 10 mg/day of (R)-Compound 1 to the human.

Aspect 22. The method of any one of the preceding Aspects, wherein the amount of (R)-Compound 1 is administered to the human in a single dose.

Aspect 23. The method of any one of the preceding Aspects, wherein the amount of (R)-Compound 1 is administered to the human in a divided dose, for example, in two doses, three doses, or four doses.

Aspect 24. The method of any one of the preceding Aspects, wherein the (R)-Compound is administered while the human is in a fasted state.

Aspect 25. The method of any one of the preceding Aspects, wherein the administration of the (R)-Compound 1 does not result in a clinically significant adverse event in the human.

Aspect 26. The method of any one of the preceding Aspects, wherein the human is at least 18 years of age.

Aspect 27. The method of any one of the preceding Aspects, wherein hypertension is treated.

Aspect 28. The method of any one of the preceding Aspects, wherein primary aldosteronism is treated.

Aspect 29. A pharmaceutical composition comprising about 0.5 to about 10 mg of (R)-Compound 1 and a pharmaceutically acceptable excipient.

What is claimed is:

1. A method of treating hypertension or primary aldosteronism in a human having a mean seated blood pressure of ≥130/80 mmHg, comprising administering 0.1 to 10 mg/day of (R)-Compound 1 to the human:

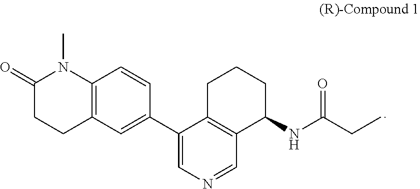 [SMILES: CCC(=O)N[C@@H]1CCCc2c(-c3ccc4c(c3)CCC(=O)N4C)cncc21]

2. The method of claim 1, wherein the human is on a stable background hypertensive regimen prior to the administration of the (R)-Compound 1.

3. The method of claim 2, wherein the stable background hypertensive regimen comprising ≥3 antihypertensive agents.

4. The method of claim 3, wherein one of the antihypertensive agents is a diuretic.

5. The method of any one of the preceding claims, wherein the administration of the (R)-Compound 1 results in a mean decrease from baseline in seated systolic blood pressure (SBP) after 4 weeks of treatment.

6. The method of any one of the preceding claims, wherein the administration of the (R)-Compound 1 results in a mean decrease from baseline in seated systolic blood pressure (SBP) after 12 weeks of treatment.

7. The method of any one of the preceding claims, wherein the administration of the (R)-Compound 1 results in a mean decrease in baseline in seated diastolic blood pressure (DBP) after 4 weeks of treatment.

8. The method of any one of the preceding claims, wherein the administration of the (R)-Compound 1 results in a mean decrease in baseline in seated diastolic blood pressure (DBP) after 12 weeks of treatment.

9. The method of any one of the preceding claims, wherein the administration of the (R)-Compound 1 results in a seated blood pressure (BP) of <130/80 mmHg after 4 weeks of treatment.

10. The method of any one of the preceding claims, wherein the administration of the (R)-Compound 1 results in a seated blood pressure (BP) of <130/80 mmHg after 12 weeks of treatment.

11. The method of any one of the preceding claims comprising administering about 0.5 mg/day of (R)-Compound 1 to the human.

12. The method of any one of claims 1 to 10, comprising administering about 1 mg/day of (R)-Compound 1 to the human.

13. The method of any one of claims 1 to 10, comprising administering about 2 mg/day of (R)-Compound 1 to the human.

14. The method of any one of claims 1 to 10, comprising administering about 3 mg/day of (R)-Compound 1 to the human.

15. The method of any one of claims 1 to 10, comprising administering about 4 mg/day of (R)-Compound 1 to the human.

16. The method of any one of claims 1 to 10, comprising administering about 5 mg/day of (R)-Compound 1 to the human.

17. The method of any one of claims 1 to 10, comprising administering about 6 mg/day of (R)-Compound 1 to the human.

18. The method of any one of claims 1 to 10, comprising administering about 7 mg/day of (R)-Compound 1 to the human.

19. The method of any one of claims 1 to 10, comprising administering about 8 mg/day of (R)-Compound 1 to the human.

20. The method of any one of claims 1 to 10, comprising administering about 9 mg/day of (R)-Compound 1 to the human.

21. The method of any one of claims 1 to 10, comprising administering about 10 mg/day of (R)-Compound 1 to the human.

22. The method of any one of the preceding claims, wherein the amount of (R)-Compound 1 is administered to the human in a single dose.

23. The method of any one of the preceding claims, wherein the amount of (R)-Compound 1 is administered to the human in a divided dose, for example, in two doses, three doses, or four doses.

24. The method of any one of the preceding claims, wherein the (R)-Compound is administered while the human is in a fasted state.

25. The method of any one of the preceding claims, wherein the administration of the (R)-Compound 1 does not result in a clinically significant adverse event in the human.

26. The method of any one of the preceding claims, wherein the human is at least 18 years of age.

27. The method of any one of the preceding claims, wherein hypertension is treated.

28. The method of any one of the preceding claims, wherein primary aldosteronism is treated.

29. A pharmaceutical composition comprising about 0.1 to about 20 mg of Compound 1 and a pharmaceutically acceptable excipient.

30. The pharmaceutical composition of claim 29 comprising about 0.1 to about 10 mg of (R)-Compound 1 and a pharmaceutically acceptable excipient.
